(19) United States
(12) Patent Application Publication
(10) Pub. No.: US 2026/0335196 A1
(43) Pub. Date: Oct. 29, 2026

(54) HYDROGELS WITH ANTIBIOFILM, ANTIMICROBIAL, AND MINERALIZING THERAPEUTIC EFFECTS

(71) Applicant: Temple University-of The Commonwealth System of Higher Education, Philadelphia, PA (US)

(72) Inventor: Santiago Orrego, Philadelphia, PA (US)

(21) Appl. No.: 19/320,114

(22) Filed: Sep. 5, 2025

Related U.S. Application Data
(60) Provisional application No. 63/691,446, filed on Sep. 6, 2024.

(51) Int. Cl. A61K 6/15; A61C 17/00; A61K 6/54; A61K 6/70

(52) U.S. Cl. CPC A61K 6/15; A61C 17/00; A61K 6/54; A61K 6/70; A61C 2201/00

(57) ABSTRACT

The present invention provides novel multi-functional (e.g., promoting formation of minerals, having antibiofilm properties, and having antimicrobial properties) compositions and methods of use thereof. In various embodiments, the composition comprises a polymer matrix, such as a hydrogel, and a piezoelectric material. In various aspects, the present invention also provides methods of preventing or treating an oral disease, disorder, or condition (e.g., periodontal disease, peri-implantitis, oral infection, etc.) and/or promoting mineralization in a subject in need thereof using said compositions.

### CROSS-REFERENCE TO RELATED APPLICATIONS

This application claims priority to and the benefit of U.S. Provisional Patent Application No. 63/691,446, filed Sep. 6, 2024, the disclosure of which is incorporated herein by reference in its entirety.

### BACKGROUND OF THE INVENTION

Periodontal disease (PD) is a major oral disease affecting half of adults in the United States (Eke et al., 2018, Journal of the American Dental Association, 149:576-588). PD is caused by pathogenic microflora in the biofilm that forms adjacent to the teeth (Socransky et al., 1998, J. Clin Periodontol., 25:134-144). The bacterial infection induces an inflammatory response that triggers the progressive destruction of the periodontal tissues and, finally, the loss of teeth (Fi et al., 2021, J. Biol. Regul Homeostatic Agents, 35:1-9). The clinical treatment of PD frequently begins with a nonsurgical approach that includes scaling and root planning (SRP) in combination with the administration of adjuvants (local or systemic antibiotics) (Jepsen et al., 2016, Periodontol. 2000, 71:82-112; Graziani et al., 2017, Periodontol. 2000, 75:152-188). The aim of nonsurgical treatment of PD is to halt the progression of the disease, control infection, and restore gum health without the need for invasive surgical procedures (Darby et al., 2009, Aust Dent J., 54:S86-S95). Nonsurgical treatments are typically the first line of defense in managing PD. However, the clinical success of nonsurgical treatment is only 39%, with major reported inconsistencies, dependence on patient compliance, smoking status, tooth location, and disease severity (Soysa et al., 2021, Dent. Rev., 1:100006; Corbella et al., 2021, Sci. Rep., 11:1-18). The SRP plus adjuvant approach has several limitations, including the difficulty to reach pockets with greater depths, limited regeneration of periodontal tissues, limited reduction of the pocket size, and frequent reapplication and long-term use of adjuvants, which raises concerns regarding antibiotic resistance (Ardila et al., 2022, Oral Dis, doi: 10.1111/odi.14288; Davey et al., 2000, Microbiol Mol. Biol. Rev., 64:847-867; Iviglia et al., 2019, J. Funct. Biomater., 10:3). The regeneration of periodontal tissues remains challenging, especially via nonsurgical treatments with moderate/severe conditions and noncompliant patients (Bartold et al., 2016, J. Periodontal Res., 51:1-15; Ribeiro et al., 2003, Periodontol. 2000, 91:7-19). If nonsurgical treatment is ineffective, surgery may be indicated via bone grafting, guided bone tissue regeneration membranes, and scaffolds that deliver bioactive molecules (Hernández-Monjaraz et al., 2018, Int. J. Mol. Sci., 19:944; Larsson et al., 2016, J. Dent Res., 95:255-266; Bonito et al., 2005, J. Periodontol., 76:1227-1236; Shue et al., 2012, Biomatter., 2:271-277).

Thus, there is a need in the art for compositions and methods which prevent or treat oral diseases, disorders, or conditions, such as PD, and/or prevent or treat loss of tooth structure. The present invention satisfies this unmet need.

### SUMMARY OF THE INVENTION

In one aspect, the present invention provides a composition comprising a polymer matrix and a piezoelectric material.

In some embodiments, the piezoelectric material is dispersed within the polymer matrix. In some embodiments, the piezoelectric material comprises BaTiO₃ (BTO) or a derivative thereof. In some embodiments, the composition comprises between about 0.01% w/v to about 50% w/v of the piezoelectric material.

In some embodiments, the polymer matrix comprises a hydrogel. In some embodiments, the hydrogel comprises methacrylate-containing hydrogel, acrylate-containing hydrogel, gelatin-based hydrogel, wherein the gelatin-based hydrogel is optionally functionalized, or any combination thereof. In some embodiments, the hydrogel comprises gelatin methacrylate or an analog thereof, gelatin acrylate or an analog thereof, or any combination thereof.

In some embodiments, the composition a) has a shear storage modulus (G′) between about 10² Pa to about 10⁴ Pa; b) has a shear loss modulus (G″) between about 10² Pa to about 10³ Pa; c) has a voltage per surface area of at least about 9 mV/mm²; d) has an injection force of less than about 3 kgf; e) has a flow rate of about 2 mL/h; f) comprises at least one pore having an average diameter of at least about 10 μm; g) comprises a surface roughness of less than 0.2 μm; h) has curing time of between about 15 seconds to about 180 seconds; i) has a degradation rate of at least about 5% over about 14 days; or any combination thereof.

In some embodiments, the composition further comprises a nanoparticle, initiator (e.g., photoinitiator, thermal initiator, radical initiator, etc.), stabilizer, light and/or thermal emitting component, therapeutic agent, polymer, binding agent, linking molecule, or any combination thereof.

In some embodiments, the nanoparticle comprises silicon oxide nanoparticle, silanized nanoparticle, silicon-free nanoparticle, or any combination thereof.

In some embodiments, the initiator comprises a photoinitiator. In some embodiments, the photoinitiator comprises lithium phenyl-2,4,6-trimethylbenzoyl phosphonate (LAP) or a derivative thereof.

In some embodiments, the composition is a light emitting component-free composition, thermal emitting component-free composition, or a combination thereof.

In some embodiments, the composition is a biocompatible composition, biodegradable composition, antimicrobial composition, or any combination thereof.

In some embodiments, the composition is a dental composition.

In some embodiments, the piezoelectric material promotes bone growth, prevents, reduces, or treats bone damage, prevents, reduces, or treats bone structure damage, prevents, reduces, or treats tooth damage, prevents, reduces, or treats tooth structure damage, prevents, reduces, or treats an infection, prevents, reduces, or treats an inflammation (e.g., gum inflammation, gingivitis, etc.), prevents or reduces biofilm formation, or any combination thereof.

In some embodiments, the infection is a bacterial infection, fungal infection, viral infection, or any combination thereof.

In one aspect, the present invention provides a method of preventing or treating an oral disease, disorder, or condition in a subject in need thereof.

In another aspect, the present invention provides a method of simultaneously preventing or treating an oral disease, disorder, or condition and promoting mineralization in a subject in need thereof.

In some embodiments, the method comprises applying at least one composition of the present invention to at least a portion of a tooth of the subject.

In some embodiments, the oral disease, disorder, or condition is periodontal disease, peri-implantitis, oral infection, loss of tooth structure, gum inflammation, gingivitis, or any combination thereof. In some embodiments, the oral infection is a bacterial infection, fungal infection, viral infection, or any combination thereof.

In some embodiments, the subject generates mastication forces producing electrical charges at the surface of the composition, wherein the electrical charges simultaneously prevent or reduce microbial growth and precipitate new minerals.

### BRIEF DESCRIPTION OF THE DRAWINGS

The following detailed description of various embodiments of the invention will be better understood when read in conjunction with the appended drawings. For the purpose of illustrating the invention, these are shown in the drawings illustrative embodiments. It should be understood, however, that the invention is not limited to the precise arrangements and instrumentalities of the embodiments shown in the drawings.

FIG. 1 depicts a schematic representation of injectable piezoelectric hydrogel for periodontal disease treatment.

FIG. 2, comprising FIG. 2A through FIG. 2F, depicts representative characterization results of gelatin methacryloyl (GelMA) and piezoelectric hydrogel (PiezoGEL) hydrogels. FIG. 2A depicts representative storage modulus of cured hydrogels as a function of strain. FIG. 2B depicts representative loss modulus of cured hydrogels as a function of strain. FIG. 2C depicts representative injection force required for the uncured hydrogel to be extruded through a 20-G½ tip. FIG. 2D depicts representative storage modulus of GelMA hydrogel as a function of the time. Indication of polymerization. Measurements were performed at 37° C. FIG. 2E depicts representative voltage per surface area generated after being subjected to a cyclic compression load of 3 N at 2 Hz. FIG. 2F depicts representative biodegradation profiles of hydrogels placed in phosphate buffered saline (PBS) for 14 days at 37° C. Mean values with different letters are significantly different from each other (p≤0.05).

FIG. 3, comprising FIG. 3A through FIG. 3H, depicts representative results of antibacterial evaluation of GelMA and PiezoGEL hydrogels against Porphyromonas gingivalis in vitro. FIG. 3A depicts a schematic representation of the model used to cultivate biofilms on hydrogels subjected to cyclic mechanical loading and static (no load). FIG. 3B depicts representative microbiological evaluations measuring biofilm. FIG. 3C depicts representative microbiological evaluations measuring metabolic activity. FIG. 3D depicts representative microbiological evaluations measuring cell viability (or colony forming units (CFU)). FIG. 3E depicts representative CLSM images of P. gingivalis. Cells were stained with Syto-9 (green) and propidium iodide (red) to indicate live and dead bacteria, respectively. FIG. 3F depicts representative changes in the expression of selected genes related to P. gingivalis biofilm formation: porP encoding biosynthesis of cell-surface polysaccharides and biofilm growth. FIG. 3G depicts representative changes in the expression of selected genes related to P. gingivalis biofilm formation: fimA related to the adhesion of host-pathogen interactions.

FIG. 3H depicts representative changes in the expression of selected genes related to P. gingivalis biofilm formation: oxyR regulator of oxidative stress. The relative gene expression was normalized to 16S rRNA levels (housekeeping). Mean values with different letters are significantly different (p≤0.05).

FIG. 4, comprising FIG. 4A through FIG. 4F, depicts representative osteogenic differentiation evaluation of GelMA and PiezoGEL hydrogels in vitro. FIG. 4A depicts a schematic representation of the model developed to evaluate bone tissue regeneration on hydrogels subjected to cyclic mechanical loading and static (no loading). FIG. 4B depicts representative cell viability measured with alamarBlue reduction (%) and normalized with cells cultivated in an empty wells. FIG. 4C depicts representative changes in the expression of selected genes related to osteoblast differentiation of RUNX2, encoding runt related transcription factor 2 required for intramembranous and endochondral bone development. The relative gene expression was normalized to GAPDH (housekeeping). FIG. 4D depicts representative changes in the expression of selected genes related to osteoblast differentiation of COL1A1, coding collagen type I. The relative gene expression was normalized to GAPDH (housekeeping). FIG. 4E depicts representative changes in the expression of selected genes related to osteoblast differentiation of ALP, encoding alkaline phosphatase. The relative gene expression was normalized to GAPDH (housekeeping). FIG. 4F depicts representative normalized absorbance values of Alizarin Red S staining of the formed ECM minerals formed after 14 days of cell culture. Mean values with different letters are significantly different (p≤0.05).

FIG. 5, comprising FIG. 5A through FIG. 5E, depicts representative periodontal bone tissue regeneration evaluations in vivo. FIG. 5A depicts a schematic representation of the ligature-induced model in mice. A 5-0 silk ligature was tied around the maxillary second molar for 8 days to induce periodontitis. Bone regeneration was evaluated after 30 days of treatment. Treatment groups included scaling and root planning (SRP) as clinical reference, and SRP followed by the injection of both GelMA or PiezoGEL hydrogels. FIG. 5B depicts representative change of periodontal pocket depth calculated by measuring the before and after. FIG. 5C depicts representative change in cementum-enamel junction (CEJ) to alveolar bone crest (ABC) distance (yellow line). FIG. 5D depicts representative change in bone volume measured between molars 1 and 2 (ligature location) and quantified along several cut planes along the apical/occlusal direction. FIG. 5E depicts representative reconstructed 3D micro-CT images of the maxillary alveolar bone viewed from the lingual direction in all groups. Mean values with different letters are significantly different (p≤0.05).

FIG. 6, comprising FIG. 6A through FIG. 6C, depicts representative histological evaluation of animal periodontal tissues. The red arrow corresponds to epithelium detached from cement. Yellow arrows correspond to indicate blood vessels. Green and blue arrows correspond to erythrocytes and neutrophils, respectively. FIG. 6A depicts representative hematoxylin and eosin (H&E) images of periodontal tissue sections between the maxilla's first and second molars with magnification inset. FIG. 6B depicts a representative number of blood vessels quantified in the regenerated area. FIG. 6C depicts a representative number of erythrocytes quantified in the regenerated area.

FIG. 7 depicts representative biodegradation profiles of hydrogels with different filler and GelMA concentrations placed in phosphate buffered saline (PBS) for 14 days at 37° C.

FIG. 8, comprising FIG. 8A and FIG. 8B, depicts representative results of evaluations performed to select the proper filler (Barium titanate (BaTiO₃, BTO)) concentration to fabricate PiezoGEL. Means with different letters are significantly different from each other (p≤0.05). FIG. 8A depicts representative injection force required for the uncured hydrogels to be extruded through a 20-G½. FIG. 8B depicts a representative effect of filler concentration on the voltage generation of PiezoGEL.

FIG. 9 depicts representative surface pore size of GelMA and PiezoGEL hydrogels. The error bars were obtained from N=10 measurements. Means with different letters are significantly different from each other (p≤0.05).

FIG. 10 depicts representative surface roughness (Ra) for GelMA and PiezoGEL hydrogels. The error bars were obtained from N=7 measurements. Means with different letters are significantly different from each other (p≤0.05).

FIG. 11, comprising FIG. 11A through FIG. 11C, depicts representative images for method for quantification of live and dead cells using Image J. FIG. 11A depicts representative Image Splitting: the images are split into two channels, live (Green) and dead (Red). FIG. 11B depicts representative results of 8-bit conversion and histogram analysis where each channel is converted to 8-bit and a histogram analysis is performed. This enabled the quantification of the percentage of bacteria present in each channel. FIG. 11C depicts representative Image merging, LIVE and DEAD images were merged back together, combining the quantified information of the bacterial presence in both channels.

FIG. 12, comprising FIG. 12A through FIG. 12F, depicts representative results of osteogenic differentiation evaluation of hydrogels and differentiation media in vitro. FIG. 12A depicts a schematic representation of the model developed to evaluate bone tissue regeneration on hydrogels subjected to cyclic mechanical loading and static (no loading). FIG. 12B depicts representative results of cell viability measured with alamarBlue reduction (%) and normalized with cells cultivated in an empty wells. FIG. 12C depicts representative changes in the expression of selected genes related to osteoblast differentiation of RUNX2, encoding runt-related transcription factor 2 required for intramembranous and endochondral bone development. The relative gene expression was normalized to GAPDH (housekeeping). FIG. 12D depicts representative changes in the expression of selected genes related to osteoblast differentiation of COL1A1, coding collagen type I. The relative gene expression was normalized to GAPDH (housekeeping). FIG. 12E depicts representative changes in the expression of selected genes related to osteoblast differentiation of ALP, encoding alkaline phosphatase. The relative gene expression was normalized to GAPDH (housekeeping). FIG. 12F depicts representative normalized absorbance values of Alizarin Red S staining of the formed ECM minerals formed after 14 days of cell culture. Means with different letters are significantly different (p≤0.05).

FIG. 13 depicts representative alizarin Red S concentration versus absorbance at 405 nm. Each concentration was evaluated in triplicates.

FIG. 14 depicts representative epithelial length measured for all evaluated treatments. Representative histological images showing the changes in the epithelia. The yellow line represents the epithelial length measured.

FIG. 15, comprising FIG. 15A and FIG. 15B, depicts representative results of inflammation assessment performed by two-blinded pathologists. The inflammatory response was evaluated in five groups: No treatment, SRP, GelMA, PiezoGEL, and healthy. A bar graph is presented to show the inflammation assessed by pathologists. FIG. 15A depicts representative results of inflammation that was evaluated in terms of lymphoplasmacytic infiltration. FIG. 15B depicts representative results of inflammation that was evaluated in terms of congestion.

FIG. 16 depicts representative antibacterial activity of PiezoGEL measured by counting the number of viable cells. A significantly lower cell count can be observed in the loaded PiezoGEL.

FIG. 17, comprising FIG. 17A through FIG. 17C, depicts representative results demonstrating piezoGEL BTO filler leaching. FIG. 17A depicts representative scanning electron micrograph images of the collected material after PiezoGEL was incubated for 7 days under cyclic loading in cell growth media. FIG. 17B depicts representative scanning electron micrograph images of the collected material after PiezoGEL was incubated for 7 days under static loading in cell growth media. FIG. 17C depicts representative EDS spectrum of the collected material showing peaks corresponding to barium and titanium of the BTO.

FIG. 18 depicts representative comparison of CEJ-ABC distance in healthy periodontal tissues and ligature-induced periodontal disease in the experiment (PD) and the literature (PDCTRL).

FIG. 19 depicts representative results of bone defect model demonstrating PiezoGEL™ promoted regrowth of high-quality bone.

### DETAILED DESCRIPTION

The present invention is based, in part, on the development of a novel composition having multi-functions of repelling biofilms, promoting formation of minerals, and having antimicrobial properties. In one aspect, the composition comprises a polymer matrix and a piezoelectric material. In one embodiment, the polymer matrix comprises a hydrogel. In one embodiment, the piezoelectric material is BaTiO₃ (BTO) or a derivative thereof. In some embodiments, the composition further comprises a nanoparticle, initiator (e.g., photoinitiator, thermal initiator, radical initiator, etc.), light and/or thermal emitting component, or any combination thereof. In one embodiment, the composition is a biocompatible composition, biodegradable composition, antimicrobial composition, or any combination thereof. In some embodiments, the composition promotes bone growth, prevents, reduces, or treats bone damage and/or bone structure damage, prevents, reduces, or treats tooth damage and/or tooth structure damage, prevents, reduces, or treats an infection, prevents, reduces, or treats an inflammation (e.g., gum inflammation, gingivitis, etc.), prevents or reduces a biofilm formation, or any combination thereof. Thus, in some embodiments, the composition is used in medical applications (e.g., dentistry, orthopedics, medical devices, basic research, wound healing, dental composites, implant, titanium implant, coatings, such as surface coating of a titanium implant, etc.).

In one aspect, the present invention provides a method of preventing or treating an oral disease, disorder, or condition, preventing or reducing biofilms, promoting mineralization, or any combination thereof. In some embodiments, the method comprises applying the composition of the present invention to a subject in need thereof. In one embodiment, the composition is applied to a tooth. In some embodiments, the subject generates mastication forces producing electrical charges at the surface of the composition, wherein the electrical charges simultaneously prevent or reduce microbial growth and precipitate new minerals.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

As used herein, the term “polymer” refers to a molecule composed of repeating structural units typically connected by covalent chemical bonds. The term “polymer” is also meant to include the terms copolymer and oligomers. In one embodiment, a polymer comprises a backbone (i.e., the chemical connectivity that defines the central chain of the polymer, including chemical linkages among the various polymerized monomeric units) and a side chain (i.e., the chemical connectivity that extends away from the backbone).

The term “copolymer” in the sense of the invention refers to a polymer whose constituent units are derived from more than one type of monomer.

As used herein, the term “nanoparticle” refers to particles having a particle size on the nanometer scale, less than 1 micrometer. For example, the nanoparticle may have a particle size up to about 50 nm. In another example, the nanoparticle may have a particle size up to about 10 nm. In another example, the nanoparticle may have a particle size up to about 6 nm. As used herein, “nanoparticle” refers to a number of nanoparticles, including, but not limited to, nanoclusters, nanocapsules, core-shell nanocapsules, nanovesicles, micelles, block copolymer micelles, lamaellae shaped particles, polymersomes, dendrimers, and other nano-size particles of various other small fabrications that are known to those of skill in the art. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

The term “hydrogel” as used herein refers to a three-dimensional, hydrophilic or amphiphilic polymeric network capable of taking up large quantities of water. The networks are composed of homopolymers or copolymers, and are insoluble due to the presence of covalent chemical or physical (ionic, hydrophobic interactions, entanglements) crosslinks. The crosslinks provide the network structure and physical integrity. Hydrogels exhibit a thermodynamic compatibility with water that allow them to swell in aqueous media.

As used herein, the term “substituted” or “functionalized” means that an atom or group of atoms has replaced hydrogen as the substituent attached to another group. The term “substituted” or “functionalized” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. The term “substituted” or “functionalized” refers for example to the substitution of a hydrogen atom in a given structure with a radical selected from the group consisting of thiol, disulfide, amino, amido, azido, imino, acrylate, methacrylate, sulfone, vinyl, vinyl sulfone, hydroxyl, hydrazone, hydrazine, hydrazide, adipic acid dihydrazide, aldehyde, carboxyl, norbornene, alkyl, heteroalkyl, aryl, heteroaryl, a polar group, a halogen, halogen alkyl, etc. When more than one position can be substituted, the substituents may be the same or different in each position.

As used herein, the term “optionally substituted” or “optionally functionalized” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

For example, in one embodiment, the substituents are independently selected from the group consisting of thiol, disulfide, amino, amido, azido, imino, acrylate, methacrylate, sulfone, vinyl, vinyl sulfone, hydroxyl, hydrazone, hydrazine, hydrazide, aldehyde, carboxyl, oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, S(═O)₂N[H, alkyl, or aryl], —C(═O)NH[substituted or unsubstituted alkyl, or substituted or unsubstituted phenyl], —C(═O)N[H or substituted or unsubstituted alkyl or aryl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH) [substituted or unsubstituted alkyl]₂, and —C(NH₂)[substituted or unsubstituted alkyl]₂. In another embodiment, by way of example, an optional substituent is selected from thiol, disulfide, amino, amido, hydroxyl, hydrazone, hydrazine, hydrazide, adipic acid dihydrazide, aldehyde, carboxyl, norbornene, oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of thiol, disulfide, amido, hydroxyl, hydrazone, hydrazine, hydrazide, adipic acid dihydrazide, aldehyde, carboxyl, norbornene, C₁₋₆ alkyl, —OH, C₁₋₆ alkoxy, halo, amino, acetamido, oxo and nitro. As used herein, where a substituent is an alkyl or alkoxy group, the carbon chain may be branched, straight or cyclic.

The terms “effective amount” and “pharmaceutically effective amount” refer to a sufficient amount of an agent to provide the desired biological result. That result can be reduction and/or alleviation of a sign, symptom, or cause of a disease, disorder, or condition, or any other desired alteration of a biological system. An appropriate effective amount in any individual case may be determined by one of ordinary skill in the art using routine experimentation.

The term “therapeutic” as used herein means a treatment and/or prophylaxis. A therapeutic effect is obtained by suppression, diminution, remission, prevention, or eradication of at least one sign or symptom of a disease, disorder, or condition.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder, disease, or condition being treated. The therapeutically effective amount will vary depending on the compound, the disease, disorder, or condition and its severity and the age, weight, etc., of the subject to be treated.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The term “pharmaceutically acceptable salt” refers to any pharmaceutically acceptable salt, which upon administration to the subject is capable of providing (directly or indirectly) a compound as described herein. Such salts are, for example, acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is a human. In various embodiments, the subject is a human subject, and may be of any race, sex, and age.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease, disorder, or condition is “alleviated” if the severity of at least one sign or symptom of the disease, disorder, or condition, the frequency with which such a sign or symptom is experienced by a patient, or both, is reduced.

The term “inhibit,” as used herein, means to suppress or block an activity or function by at least about ten percent relative to a control value. In various embodiments, the activity is suppressed or blocked by at least 50% compared to a comparator value, or by at least 55%, or by at least 60%, or by at least 65%, or by at least 70%, or by at least 75%, or by at least 80%, or by at least 85%, or by at least 90%, or by at least 95%.

To “treat” a disease, disorder, or condition as the term is used herein, means to reduce the frequency and/or severity of at least one sign or symptom of a disease, disorder, or condition experienced by a subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the development of a novel composition having multi-functions of repelling biofilms, promoting formation of minerals, and having antimicrobial properties. In one aspect, the composition comprises a polymer matrix and a piezoelectric material. In one embodiment, the polymer matrix comprises a hydrogel. In one embodiment, the piezoelectric material is BaTiO₃ (BTO) or a derivative thereof. In some embodiments, the composition further comprises a nanoparticle, initiator (e.g., photoinitiator, thermal initiator, radical initiator, etc.), light and/or thermal emitting component, or any combination thereof. In one embodiment, the composition is a biocompatible composition, biodegradable composition, antimicrobial composition, or any combination thereof. In some embodiments, the composition promotes bone growth, prevents, reduces, or treats bone damage and/or bone structure damage, prevents, reduces, or treats tooth damage and/or tooth structure damage, prevents, reduces, or treats an infection, prevents, reduces, or treats an inflammation (e.g., gum inflammation, gingivitis, etc.), prevents or reduces a biofilm formation, or any combination thereof. Thus, in some embodiments, the composition is used in medical applications (e.g., dentistry, orthopedics, medical devices, basic research, wound healing, dental composites, implant, titanium implant, coatings, such as surface coating of a titanium implant, etc.).

In one aspect, the present invention provides a method of preventing or treating an oral disease, disorder, or condition, preventing or reducing biofilms, promoting mineralization, or any combination thereof. In some embodiments, the method comprises applying the composition of the present invention to a subject in need thereof. In one embodiment, the composition is applied to a tooth. In some embodiments, the subject generates mastication forces producing electrical charges at the surface of the composition, wherein the electrical charges simultaneously prevent or reduce microbial growth and precipitate new minerals.

### Composition

In one aspect, the present invention provides a composition comprising a polymer matrix and a piezoelectric material. In one embodiment, the composition is a multi-functional composition. In some embodiments, the composition possesses antibiofilm properties, antimicrobial properties (e.g., antibacterial properties, antifungal properties, antiviral properties, etc.), anti-inflammatory properties, mineralizing properties, or any combination thereof. Thus, in some embodiments, the composition comprises an antibiofilm composition, antimicrobial composition (e.g., antibacterial composition, antifungal composition, antiviral composition, etc.), anti-inflammatory composition, mineralizing composition, or any combination thereof.

In some embodiments, the composition comprises between about 0.01 wt % to about 99.99 wt % of the piezoelectric material. In some embodiments, the composition comprises between about 0.01 wt % to about 90 wt % of the piezoelectric material. In some embodiments, the composition comprises between about 0.01 wt % to about 80 wt % of the piezoelectric material. In some embodiments, the composition comprises between about 0.01 wt % to about 70 wt % of the piezoelectric material. In some embodiments, the composition comprises between about 0.01 wt % to about 60 wt % of the piezoelectric material. In some embodiments, the composition comprises between about 0.01 wt % to about 50 wt % of the piezoelectric material. In some embodiments, the composition comprises between about 0.01 wt % to about 40 wt % of the piezoelectric material. In some embodiments, the composition comprises between about 0.01 wt % to about 30 wt % of the piezoelectric material. In some embodiments, the composition comprises between about 0.01 wt % to about 20 wt % of the piezoelectric material. In some embodiments, the composition comprises between about 0.01 wt % to about 10 wt % of the piezoelectric material. In some embodiments, the composition comprises between about 0.1 wt % to about 10 wt % of the piezoelectric material. In some embodiments, the composition comprises between about 0.01 wt % to about 1 wt % of the piezoelectric material. In some embodiments, the composition comprises between about 0.1 wt % to about 1 wt % of the piezoelectric material. For example, in some embodiments, the composition comprises between about 0.01 wt % to about 50 wt % of the piezoelectric material. In some embodiments, the composition comprises between about 0.01 wt % to about 10 wt % of the piezoelectric material. In some embodiments, the composition comprises between about 0.5 wt % to about 5 wt % of the piezoelectric material. In some embodiments, the composition comprises between about 0.01 wt % to about 1 wt % of the piezoelectric material.

In some embodiments, the composition comprises at least about 0.01 wt % of the piezoelectric material. In some embodiments, the composition comprises at least about 0.02 wt % of the piezoelectric material. In some embodiments, the composition comprises at least about 0.03 wt % of the piezoelectric material. In some embodiments, the composition comprises at least about 0.04 wt % of the piezoelectric material. In some embodiments, the composition comprises at least about 0.05 wt % of the piezoelectric material. In some embodiments, the composition comprises at least about 0.06 wt % of the piezoelectric material. In some embodiments, the composition comprises at least about 0.07 wt % of the piezoelectric material. In some embodiments, the composition comprises at least about 0.08 wt % of the piezoelectric material. In some embodiments, the composition comprises at least about 0.09 wt % of the piezoelectric material. In some embodiments, the composition comprises at least about 0.1 wt % of the piezoelectric material. In some embodiments, the composition comprises at least about 0.2 wt % of the piezoelectric material. In some embodiments, the composition comprises at least about 0.25 wt % of the piezoelectric material. In some embodiments, the composition comprises at least about 0.5 wt % of the piezoelectric material. In some embodiments, the composition comprises at least about 1 wt % of the piezoelectric material. In some embodiments, the composition comprises at least about 2 wt % of the piezoelectric material. In some embodiments, the composition comprises at least about 3 wt % of the piezoelectric material. In some embodiments, the composition comprises at least about 4 wt % of the piezoelectric material. In some embodiments, the composition comprises at least about 5 wt % of the piezoelectric material. In some embodiments, the composition comprises at least about 6 wt % of the piezoelectric material. In some embodiments, the composition comprises at least about 7 wt % of the piezoelectric material. In some embodiments, the composition comprises at least about 8 wt % of the piezoelectric material. In some embodiments, the composition comprises at least about 9 wt % of the piezoelectric material. In some embodiments, the composition comprises at least about 10 wt % of the piezoelectric material. In some embodiments, the composition comprises at least about 20 wt % of the piezoelectric material. In some embodiments, the composition comprises at least about 30 wt % of the piezoelectric material. In some embodiments, the composition comprises at least about 40 wt % of the piezoelectric material. In some embodiments, the composition comprises at least about 50 wt % of the piezoelectric material. In some embodiments, the composition comprises at least about 60 wt % of the piezoelectric material. In some embodiments, the composition comprises at least about 70 wt % of the piezoelectric material. In some embodiments, the composition comprises at least about 80 wt % of the piezoelectric material. In some embodiments, the composition comprises at least about 90 wt % of the piezoelectric material. In some embodiments, the composition comprises at least about 99.99 wt % of the piezoelectric material.

In some embodiments, the composition comprises between about 0.01% w/v to about 99.99% w/v of the piezoelectric material. In some embodiments, the composition comprises between about 0.01% w/v to about 90% w/v of the piezoelectric material. In some embodiments, the composition comprises between about 0.01% w/v to about 80% w/v of the piezoelectric material. In some embodiments, the composition comprises between about 0.01% w/v to about 70% w/v of the piezoelectric material. In some embodiments, the composition comprises between about 0.01% w/v to about 60% w/v of the piezoelectric material. In some embodiments, the composition comprises between about 0.01% w/v to about 50% w/v of the piezoelectric material. In some embodiments, the composition comprises between about 0.01% w/v to about 40% w/v of the piezoelectric material. In some embodiments, the composition comprises between about 0.01% w/v to about 30% w/v of the piezoelectric material. In some embodiments, the composition comprises between about 0.01% w/v to about 20% w/v of the piezoelectric material. In some embodiments, the composition comprises between about 0.01% w/v to about 10% w/v of the piezoelectric material. In some embodiments, the composition comprises between about 0.1% w/v to about 10% w/v of the piezoelectric material. In some embodiments, the composition comprises between about 0.01% w/v to about 1% w/v of the piezoelectric material. In some embodiments, the composition comprises between about 0.1% w/v to about 1% w/v of the piezoelectric material. For example, in some embodiments, the composition comprises between about 0.01% w/v to about 50% w/v of the piezoelectric material. In some embodiments, the composition comprises between about 0.01% w/v to about 10% w/v of the piezoelectric material. In some embodiments, the composition comprises between about 0.5% w/v to about 5% w/v of the piezoelectric material. In some embodiments, the composition comprises between about 0.01% w/v to about 1% w/v of the piezoelectric material.

In some embodiments, the composition comprises at least about 0.01% w/v of the piezoelectric material. In some embodiments, the composition comprises at least about 0.02% w/v of the piezoelectric material. In some embodiments, the composition comprises at least about 0.03% w/v of the piezoelectric material. In some embodiments, the composition comprises at least about 0.04% w/v of the piezoelectric material. In some embodiments, the composition comprises at least about 0.05% w/v of the piezoelectric material. In some embodiments, the composition comprises at least about 0.06% w/v of the piezoelectric material. In some embodiments, the composition comprises at least about 0.07% w/v of the piezoelectric material. In some embodiments, the composition comprises at least about 0.08% w/v of the piezoelectric material. In some embodiments, the composition comprises at least about 0.09% w/v of the piezoelectric material. In some embodiments, the composition comprises at least about 0.1% w/v of the piezoelectric material. In some embodiments, the composition comprises at least about 0.2% w/v of the piezoelectric material. In some embodiments, the composition comprises at least about 0.25% w/v of the piezoelectric material. In some embodiments, the composition comprises at least about 0.5% w/v of the piezoelectric material. In some embodiments, the composition comprises at least about 1% w/v of the piezoelectric material. In some embodiments, the composition comprises at least about 2% w/v of the piezoelectric material. In some embodiments, the composition comprises at least about 3% w/v of the piezoelectric material. In some embodiments, the composition comprises at least about 4% w/v of the piezoelectric material. In some embodiments, the composition comprises at least about 5% w/v of the piezoelectric material. In some embodiments, the composition comprises at least about 6% w/v of the piezoelectric material. In some embodiments, the composition comprises at least about 7% w/v of the piezoelectric material. In some embodiments, the composition comprises at least about 8% w/v of the piezoelectric material. In some embodiments, the composition comprises at least about 9% w/v of the piezoelectric material. In some embodiments, the composition comprises at least about 10% w/v of the piezoelectric material. In some embodiments, the composition comprises at least about 20% w/v of the piezoelectric material. In some embodiments, the composition comprises at least about 30% w/v of the piezoelectric material. In some embodiments, the composition comprises at least about 40% w/v of the piezoelectric material. In some embodiments, the composition comprises at least about 50% w/v of the piezoelectric material. In some embodiments, the composition comprises at least about 60% w/v of the piezoelectric material. In some embodiments, the composition comprises at least about 70% w/v of the piezoelectric material. In some embodiments, the composition comprises at least about 80% w/v of the piezoelectric material. In some embodiments, the composition comprises at least about 90% w/v of the piezoelectric material. In some embodiments, the composition comprises at least about 99.99% w/v of the piezoelectric material. For example, in some embodiments, the composition comprises at least about 5% w/v of the piezoelectric material. In some embodiments, the composition comprises at least about 9% w/v of the piezoelectric material.

In some embodiments, the piezoelectric material comprises a piezoelectric particle having a diameter of between about 10 nm to about 500 nm. In some embodiments, the piezoelectric particle has a diameter of between about 10 nm to about 400 nm. In some embodiments, the piezoelectric particle has a diameter of between about 10 nm to about 300 nm. In some embodiments, the piezoelectric particle has a diameter of between about 10 nm to about 200 nm. In some embodiments, the piezoelectric particle has a diameter of between about 10 nm to about 100 nm. In some embodiments, the piezoelectric particle has a diameter of between about 20 nm to about 500 nm. In some embodiments, the piezoelectric particle has a diameter of between about 30 nm to about 500 nm. In some embodiments, the piezoelectric particle has a diameter of between about 40 nm to about 500 nm. In some embodiments, the piezoelectric particle has a diameter of between about 50 nm to about 500 nm. In some embodiments, the piezoelectric particle has a diameter of between about 100 nm to about 500 nm. In some embodiments, the piezoelectric particle has a diameter of between about 50 nm to about 450 nm. In some embodiments, the piezoelectric particle has a diameter of between about 50 nm to about 400 nm. In some embodiments, the piezoelectric particle has a diameter of between about 50 nm to about 300 nm. In some embodiments, the piezoelectric particle has a diameter of between about 50 nm to about 200 nm. In some embodiments, the piezoelectric particle has a diameter of between about 100 nm to about 200 nm. For example, in some embodiments, the piezoelectric particle has a diameter of between about 50 nm to about 300 nm. In some embodiments, the piezoelectric particle has a diameter of about 200 nm.

In some embodiments, the piezoelectric material comprises a silicon-containing piezoelectric material. For example, in one embodiment, the piezoelectric material comprises silicon oxide.

In some embodiments, the piezoelectric material comprises silicon-free piezoelectric material.

In some embodiments, the piezoelectric material comprises BaTiO₃ (BTO) or a derivative thereof, langasite (La₃Ga₅SiO₁₄) or a derivative thereof, gallium orthophosphate (GaPO₄) or a derivative thereof, lithium niobate (LiNbO₃) or a derivative thereof, lithium tantalate (LiTaO₃) or a derivative thereof, quartz or a derivative thereof, berlinite (AlPO₄) or a derivative thereof, rochelle salt or a derivative thereof, topaz or a derivative thereof, tourmaline-group mineral or a derivative thereof, lead titanate (PbTiO₃) or a derivative thereof, lead zirconate titanate (Pb[ZrₓTi₁₋ₓ]O₃ with 0≤x≤1) or a derivative thereof, potassium niobate (KNbO₃) or a derivative thereof, sodium tungstate (Na₂WO₃) or a derivative thereof, Ba₂NaNb₅O₅ or a derivative thereof, Pb₂KNb₅O₁₅ or a derivative thereof, zinc oxide (ZnO) or a derivative thereof, sodium potassium niobate ((K,Na)NbO₃) or a derivative thereof, bismuth ferrite (BiFeO₃) or a derivative thereof, sodium niobate (NaNbO₃) or a derivative thereof, bismuth titanate (Bi₄Ti₃O₁₂) or a derivative thereof, sodium bismuth titanate (NaBi(TiO₃)₂) or a derivative thereof, polyvinylidene fluoride (PVDF) or a derivative thereof, sucrose or a derivative thereof, or any combination thereof. For example, in one embodiment, the piezoelectric material comprises BaTiO₃ (BTO) or a derivative thereof.

In one embodiment, the polymer matrix comprises a resin matrix.

In one embodiment, the polymer matrix comprises a gel. In one embodiment, the composition is a gel. In one embodiment, the gel is a hydrogel. Thus, in one embodiment, the polymer matrix comprises a hydrogel. In one embodiment, the composition is a hydrogel.

In some embodiments, the composition comprises between about 0.01 wt % to about 99.99 wt % of the hydrogel. In some embodiments, the composition comprises between about 0.01 wt % to about 90 wt % of the hydrogel. In some embodiments, the composition comprises between about 0.01 wt % to about 80 wt % of the hydrogel. In some embodiments, the composition comprises between about 0.01 wt % to about 70 wt % of the hydrogel. In some embodiments, the composition comprises between about 0.01 wt % to about 60 wt % of the hydrogel. In some embodiments, the composition comprises between about 0.01 wt % to about 50 wt % of the hydrogel. In some embodiments, the composition comprises between about 0.01 wt % to about 40 wt % of the hydrogel. In some embodiments, the composition comprises between about 0.01 wt % to about 30 wt % of the hydrogel. In some embodiments, the composition comprises between about 0.01 wt % to about 20 wt % of the hydrogel. In some embodiments, the composition comprises between about 1 wt % to about 30 wt % of the hydrogel. In some embodiments, the composition comprises between about 10 wt % to about 20 wt % of the hydrogel. In some embodiments, the composition comprises between about 15 wt % to about 25 wt % of the hydrogel. For example, in some embodiments, in some embodiments, the composition comprises between about 15 wt % to about 20 wt % of the hydrogel.

In some embodiments, the composition comprises at least about 0.01 wt % of the hydrogel. In some embodiments, the composition comprises at least about 0.5 wt % of the hydrogel. In some embodiments, the composition comprises at least about 1 wt % of the hydrogel. In some embodiments, the composition comprises at least about 5 wt % of the hydrogel. In some embodiments, the composition comprises at least about 10 wt % of the hydrogel. In some embodiments, the composition comprises at least about 15 wt % of the hydrogel. In some embodiments, the composition comprises at least about 20 wt % of the hydrogel. In some embodiments, the composition comprises at least about 25 wt % of the hydrogel. In some embodiments, the composition comprises at least about 30 wt % of the hydrogel. In some embodiments, the composition comprises at least about 40 wt % of the hydrogel. In some embodiments, the composition comprises at least about 50 wt % of the hydrogel. In some embodiments, the composition comprises at least about 60 wt % of the hydrogel. In some embodiments, the composition comprises at least about 70 wt % of the hydrogel. In some embodiments, the composition comprises at least about 80 wt % of the hydrogel. In some embodiments, the composition comprises at least about 90 wt % of the hydrogel. In some embodiments, the composition comprises at least about 99.99 wt % of the hydrogel. For example, in some embodiments, in some embodiments, the composition comprises about 20 wt % of the hydrogel.

In some embodiments, the composition comprises between about 0.01% w/v to about 99.99% w/v of the hydrogel. In some embodiments, the composition comprises between about 0.01% w/v to about 90% w/v of the hydrogel. In some embodiments, the composition comprises between about 0.01% w/v to about 80% w/v of the hydrogel. In some embodiments, the composition comprises between about 0.01% w/v to about 70% w/v of the hydrogel. In some embodiments, the composition comprises between about 0.01% w/v to about 60% w/v of the hydrogel. In some embodiments, the composition comprises between about 0.01% w/v to about 50% w/v of the hydrogel. In some embodiments, the composition comprises between about 0.01% w/v to about 40% w/v of the hydrogel. In some embodiments, the composition comprises between about 0.01% w/v to about 30% w/v of the hydrogel. In some embodiments, the composition comprises between about 0.01% w/v to about 20% w/v of the hydrogel. In some embodiments, the composition comprises between about 1% w/v to about 30% w/v of the hydrogel. In some embodiments, the composition comprises between about 10% w/v to about 20% w/v of the hydrogel. In some embodiments, the composition comprises between about 15% w/v to about 25% w/v of the hydrogel. For example, in some embodiments, in some embodiments, the composition comprises between about 15% w/v to about 20% w/v of the hydrogel.

In some embodiments, the composition comprises at least about 0.01% w/v of the hydrogel. In some embodiments, the composition comprises at least about 0.5% w/v of the hydrogel. In some embodiments, the composition comprises at least about 1% w/v of the hydrogel. In some embodiments, the composition comprises at least about 5% w/v of the hydrogel. In some embodiments, the composition comprises at least about 10% w/v of the hydrogel. In some embodiments, the composition comprises at least about 15% w/v of the hydrogel. In some embodiments, the composition comprises at least about 20% w/v of the hydrogel. In some embodiments, the composition comprises at least about 25% w/v of the hydrogel. In some embodiments, the composition comprises at least about 30% w/v of the hydrogel. In some embodiments, the composition comprises at least about 40% w/v of the hydrogel. In some embodiments, the composition comprises at least about 50% w/v of the hydrogel. In some embodiments, the composition comprises at least about 60% w/v of the hydrogel. In some embodiments, the composition comprises at least about 70% w/v of the hydrogel. In some embodiments, the composition comprises at least about 80% w/v of the hydrogel. In some embodiments, the composition comprises at least about 90% w/v of the hydrogel. In some embodiments, the composition comprises at least about 99.99% w/v of the hydrogel. For example, in some embodiments, in some embodiments, the composition comprises about 20% w/v of the hydrogel.

In some embodiments, the hydrogel is a viscoelastic hydrogel.

In some embodiments, the hydrogel comprises an optionally functionalized hydrogel.

In some embodiments, the hydrogel comprises a polymerizable hydrogel. In some embodiments, the hydrogel comprises a hydrogel that is not polymerizable.

In some embodiments, the hydrogel comprises a curable hydrogel. For example, in some embodiments, the curable hydrogel comprises a photocurable hydrogel, thermocurable hydrogel, air-curable hydrogel, or any combination thereof.

In some embodiments, the hydrogel comprises methacrylate-containing hydrogel, acrylate-containing hydrogel, epoxy-containing hydrogel, gelatin-based hydrogel, or any combination thereof. For example, in one embodiment, the gelatin-based hydrogel is an optionally functionalized gelatin-based hydrogel.

In some embodiments, the hydrogel comprises gelatin methacrylate or an analog thereof, gelatin acrylate or an analog thereof, gelatin epoxide, or any combination thereof.

In some embodiments, the hydrogel comprises between about 1 to about 99% water by weight. In some embodiments, the hydrogel comprises between about 1 to about 90% water by weight. In some embodiments, the hydrogel comprises between about 5 to about 90% water by weight. In some embodiments, the hydrogel comprises between about 10 to about 80% water by weight. In some embodiments, the hydrogel comprises between about 20 to about 70% water by weight. In some embodiments, the hydrogel comprises between about 30 to about 60% water by weight. In some embodiments, the hydrogel comprises between about 40 to about 50% water by weight. In some embodiments, the hydrogel comprises between about 1 to about 50% water by weight. In some embodiments, the hydrogel comprises between about 1 to about 40% water by weight. In some embodiments, the hydrogel comprises between about 1 to about 30% water by weight. In some embodiments, the hydrogel comprises between about 1 to about 20% water by weight. In some embodiments, the hydrogel comprises between about 1 to about 10% water by weight. In some embodiments, the hydrogel comprises between about 1 to about 5% water by weight. In some embodiments, the hydrogel comprises between about 5 to about 10% water by weight. In some embodiments, the hydrogel comprises between about 10 to about 20% water by weight. In some embodiments, the hydrogel comprises between about 15 to about 20% water by weight. In some embodiments, the hydrogel comprises about 20% water by weight.

In some embodiments, the hydrogen ion concentration of the hydrogel may have a pH of about 0 to about 14. In some embodiments, the hydrogen ion concentration of the hydrogel may have a pH of about 4 to about 10. In some embodiments, the hydrogen ion concentration of the hydrogel may have a pH of about 5 to about 9. In some embodiments, the hydrogen ion concentration of the hydrogel may have a pH of about 6 to about 8. In some embodiments, the hydrogen ion concentration of the hydrogel may have a pH of about 7 to about 10. In some embodiments, the hydrogen ion concentration of the hydrogel may have a pH of about 4 to about 7.

In some embodiments, the hydrogen ion concentration may be adjusted for known acids and bases.

In some embodiments, the hydrogel may further optionally comprise at least one wetting agent. In some embodiments, the wetting agent comprises a nonionic polymer, cationic polymer, glycol compound, alpha hydroxy acid, polyhydric alcohol, urea, or any combination thereof.

In some embodiments, the gel is a “chemical gel”, “physical gel”, or a combination thereof.

In some embodiments, the “chemical gel” comprises at least one covalent polymer network, e.g., a network formed by crosslinking polymer chains or by nonlinear polymerization. In some embodiments, the “chemical gel” is a supramolecular assembly whose molecules are associated by high energy bonds (covalent bonds). In some embodiments, the stability of this assembly is very large. In some embodiments, the “chemical gel” has improved chemical stability, the only way to degrade them is to destroy the covalent network bonding.

In some embodiments, the “physical gel” is a polymer network formed through the physical aggregation or supramolecular assembly of polymer chains, caused, for example, by low energy bonds such as hydrogen bonds or Van der Waals bonds, crystallization, helix formation, complexation, that results in regions of local order acting as the network junction points. In some embodiments, the stability of this assembly is associated with a specific range of physico-chemical conditions (pH, concentration of molecules, temperature, solvent quality, ionic strength, etc). Outside this range, the mixture is liquid. Thus, a change in the parameters of the medium can lead to the destruction of the scaffold and induce a gel/sol transition. In some embodiments, the “physical gel” is obtained from macromolecules or polymers of natural origin, such as proteins, polysaccharides, gelatin, alginate, pectin, and/or starch.

In some embodiments, the gel is a self-supported gel that can be dehydrated in order to be stored and transported dried.

The gel according to the present invention can thus be reversibly dehydrated and rehydrated. In some embodiments, the gel according to the invention can be dehydrated and then rehydrated between about 0.5 to about 100%, for example between about 45 to about 95%. Thus, in a particular embodiment of the present invention, here is provided a gel according to the present invention which is dehydrated and has a moisture content of between about 0.5 to 100% in weight.

The drying of the gel according to the invention can be achieved according to any technique known by the man skilled in the art. For example, the gel can be disposed into an oven at a temperature comprised between about 30 and about 100° C. for a sufficient time to evaporate the contained water and to obtain a material with a moisture content of between about 0.5 to 100% in weight.

In some embodiments, the composition has a shear storage modulus (G′) between about 100 Pa to about 100,000 Pa. In some embodiments, the composition has a shear storage modulus (G′) between about 100 Pa to about 10,000 Pa. In some embodiments, the composition has a shear storage modulus (G′) between about 100 Pa to about 5,000 Pa. In some embodiments, the composition has a shear storage modulus (G′) between about 100 Pa to about 2,000 Pa. In some embodiments, the composition has a shear storage modulus (G′) between about 1,000 Pa to about 10,000 Pa. In some embodiments, the composition has a shear storage modulus (G′) between about 2,000 Pa to about 10,000 Pa. In some embodiments, the composition has a shear storage modulus (G′) between about 3,000 Pa to about 10,000 Pa. In some embodiments, the composition has a shear storage modulus (G′) between about 4,000 Pa to about 10,000 Pa. In some embodiments, the composition has a shear storage modulus (G′) between about 5,000 Pa to about 10,000 Pa. In some embodiments, the composition has a shear storage modulus (G′) between about 6,000 Pa to about 10,000 Pa. In some embodiments, the composition has a shear storage modulus (G′) between about 7,000 Pa to about 10,000 Pa. In some embodiments, the composition has a shear storage modulus (G′) between about 8,000 Pa to about 10,000 Pa. In some embodiments, the composition has a shear storage modulus (G′) between about 9,000 Pa to about 10,000 Pa. In some embodiments, the composition has a shear storage modulus (G′) between about 10,000 Pa to about 100,000 Pa. In some embodiments, the composition has a shear storage modulus (G′) between about 20,000 Pa to about 100,000 Pa. In some embodiments, the composition has a shear storage modulus (G′) between about 25,000 Pa to about 100,000 Pa. In some embodiments, the composition has a shear storage modulus (G′) between about 50,000 Pa to about 100,000 Pa. In some embodiments, the composition has a shear storage modulus (G′) between about 80,000 Pa to about 100,000 Pa. In some embodiments, the composition has a shear storage modulus (G′) between about 90,000 Pa to about 100,000 Pa. For example, in some embodiments, the composition has a shear storage modulus (G′) between about 100 Pa to about 90,000 Pa.

In some embodiments, the composition has a shear loss modulus (G″) between about 100 Pa to about 10,000 Pa. In some embodiments, the composition has a shear loss modulus (G″) between about 100 Pa to about 5,000 Pa. In some embodiments, the composition has a shear loss modulus (G″) between about 100 Pa to about 1,000 Pa. In some embodiments, the composition has a shear loss modulus (G″) between about 100 Pa to about 2,000 Pa. In some embodiments, the composition has a shear loss modulus (G″) between about 100 Pa to about 3,000 Pa. In some embodiments, the composition has a shear loss modulus (G″) between about 100 Pa to about 4,000 Pa. In some embodiments, the composition has a shear loss modulus (G″) between about 100 Pa to about 5,000 Pa. In some embodiments, the composition has a shear loss modulus (G″) between about 100 Pa to about 6,000 Pa. In some embodiments, the composition has a shear loss modulus (G″) between about 100 Pa to about 7,000 Pa. In some embodiments, the composition has a shear loss modulus (G″) between about 100 Pa to about 8,000 Pa. In some embodiments, the composition has a shear loss modulus (G″) between about 100 Pa to about 9,000 Pa. For example, in some embodiments, the composition has a shear loss modulus (G″) between about 1,000 Pa to about 9,000 Pa.

In some embodiments, the composition has a voltage per surface area of at least about 0.01 mV/mm². In some embodiments, the composition has a voltage per surface area of at least about 0.1 mV/mm². In some embodiments, the composition has a voltage per surface area of at least about 1 mV/mm². In some embodiments, the composition has a voltage per surface area of at least about 2 mV/mm². In some embodiments, the composition has a voltage per surface area of at least about 3 mV/mm². In some embodiments, the composition has a voltage per surface area of at least about 4 mV/mm². In some embodiments, the composition has a voltage per surface area of at least about 5 mV/mm². In some embodiments, the composition has a voltage per surface area of at least about 6 mV/mm². In some embodiments, the composition has a voltage per surface area of at least about 7 mV/mm². In some embodiments, the composition has a voltage per surface area of at least about 8 mV/mm². In some embodiments, the composition has a voltage per surface area of at least about 9 mV/mm². In some embodiments, the composition has a voltage per surface area of at least about 10 mV/mm². In some embodiments, the composition has a voltage per surface area of at least about 11 mV/mm². In some embodiments, the composition has a voltage per surface area of at least about 12 mV/mm². In some embodiments, the composition has a voltage per surface area of at least about 15 mV/mm². In some embodiments, the composition has a voltage per surface area of at least about 20 mV/mm². In some embodiments, the composition has a voltage per surface area of at least about 100 mV/mm². For example, in some embodiments, the composition has a voltage per surface area of at least about 9 mV/mm².

In some embodiments, the composition has an injection force of less than about 100 kgf. In some embodiments, the composition has an injection force of less than about 10 kgf. In some embodiments, the composition has an injection force of less than about 9 kgf. In some embodiments, the composition has an injection force of less than about 8 kgf. In some embodiments, the composition has an injection force of less than about 7 kgf. In some embodiments, the composition has an injection force of less than about 6 kgf. In some embodiments, the composition has an injection force of less than about 5 kgf. In some embodiments, the composition has an injection force of less than about 4 kgf. In some embodiments, the composition has an injection force of less than about 3 kgf. In some embodiments, the composition has an injection force of less than about 2.75 kgf. In some embodiments, the composition has an injection force of less than about 2.5 kgf. In some embodiments, the composition has an injection force of less than about 2.25 kgf. In some embodiments, the composition has an injection force of less than about 2 kgf. In some embodiments, the composition has an injection force of less than about 1.9 kgf. In some embodiments, the composition has an injection force of less than about 1.8 kgf. In some embodiments, the composition has an injection force of less than about 1.7 kgf. In some embodiments, the composition has an injection force of less than about 1.6 kgf. In some embodiments, the composition has an injection force of less than about 1.5 kgf. In some embodiments, the composition has an injection force of less than about 1.4 kgf. In some embodiments, the composition has an injection force of less than about 1.3 kgf. In some embodiments, the composition has an injection force of less than about 1.2 kgf. In some embodiments, the composition has an injection force of less than about 1.1 kgf. In some embodiments, the composition has an injection force of less than about 1 kgf. In some embodiments, the composition has an injection force of less than about 0.9 kgf. In some embodiments, the composition has an injection force of less than about 0.8 kgf. In some embodiments, the composition has an injection force of less than about 0.7 kgf. In some embodiments, the composition has an injection force of less than about 0.6 kgf. In some embodiments, the composition has an injection force of less than about 0.5 kgf. In some embodiments, the composition has an injection force of less than about 0.4 kgf. In some embodiments, the composition has an injection force of less than about 0.3 kgf. In some embodiments, the composition has an injection force of less than about 0.2 kgf. In some embodiments, the composition has an injection force of less than about 0.1 kgf. For example, in some embodiments, the composition has an injection force of less than about 3 kgf. In some embodiments, the composition has an injection force of between about 0.9 kgf to about 1.2 kgf.

In some embodiments, the composition has an injection force of less than about 100 N. In some embodiments, the composition has an injection force of less than about 50 N. In some embodiments, the composition has an injection force of less than about 40 N. In some embodiments, the composition has an injection force of less than about 30 N. In some embodiments, the composition has an injection force of less than about 25 N. In some embodiments, the composition has an injection force of less than about 20 N. In some embodiments, the composition has an injection force of less than about 15 N. In some embodiments, the composition has an injection force of less than about 14 N. In some embodiments, the composition has an injection force of less than about 13 N. In some embodiments, the composition has an injection force of less than about 12 N. In some embodiments, the composition has an injection force of less than about 11 N. In some embodiments, the composition has an injection force of less than about 10 N. In some embodiments, the composition has an injection force of less than about 9 N. In some embodiments, the composition has an injection force of less than about 8 N. In some embodiments, the composition has an injection force of less than about 7 N. In some embodiments, the composition has an injection force of less than about 6 N. In some embodiments, the composition has an injection force of less than about 5 N. In some embodiments, the composition has an injection force of less than about 4 N. In some embodiments, the composition has an injection force of less than about 3 N. In some embodiments, the composition has an injection force of less than about 2 N. In some embodiments, the composition has an injection force of less than about 1 N. For example, in some embodiments, the composition has an injection force of less than about 30 N. In some embodiments, the composition has an injection force of between about 6 N to about 15 N.

In some embodiments, the composition has a flow rate of less than about 100 mL/h. In some embodiments, the composition has a flow rate of less than about 20 mL/h. In some embodiments, the composition has a flow rate of less than about 15 mL/h. In some embodiments, the composition has a flow rate of less than about 12 mL/h. In some embodiments, the composition has a flow rate of less than about 10 mL/h. In some embodiments, the composition has a flow rate of less than about 9 mL/h. In some embodiments, the composition has a flow rate of less than about 8 mL/h. In some embodiments, the composition has a flow rate of less than about 7 mL/h. In some embodiments, the composition has a flow rate of less than about 6 mL/h. In some embodiments, the composition has a flow rate of less than about 5 mL/h. In some embodiments, the composition has a flow rate of less than about 4 mL/h. In some embodiments, the composition has a flow rate of less than about 3 mL/h. In some embodiments, the composition has a flow rate of less than about 2 mL/h. In some embodiments, the composition has a flow rate of less than about 1 mL/h. For example, in some embodiments, the composition has a flow rate of about 2 mL/h.

In some embodiments, the composition comprises at least one pore having an average diameter of at least about 0.1 μm. In some embodiments, the composition comprises at least one pore having an average diameter of at least about 0.5 μm. In some embodiments, the composition comprises at least one pore having an average diameter of at least about 1 μm. In some embodiments, the composition comprises at least one pore having an average diameter of at least about 2 μm. In some embodiments, the composition comprises at least one pore having an average diameter of at least about 3 μm. In some embodiments, the composition comprises at least one pore having an average diameter of at least about 4 μm. In some embodiments, the composition comprises at least one pore having an average diameter of at least about 5 μm. In some embodiments, the composition comprises at least one pore having an average diameter of at least about 6 μm. In some embodiments, the composition comprises at least one pore having an average diameter of at least about 7 μm. In some embodiments, the composition comprises at least one pore having an average diameter of at least about 8 μm. In some embodiments, the composition comprises at least one pore having an average diameter of at least about 9 μm. In some embodiments, the composition comprises at least one pore having an average diameter of at least about 10 μm. In some embodiments, the composition comprises at least one pore having an average diameter of at least about 11 μm. In some embodiments, the composition comprises at least one pore having an average diameter of at least about 12 μm. In some embodiments, the composition comprises at least one pore having an average diameter of at least about 15 μm. In some embodiments, the composition comprises at least one pore having an average diameter of at least about 20 μm. In some embodiments, the composition comprises at least one pore having an average diameter of at least about 30 μm. In some embodiments, the composition comprises at least one pore having an average diameter of at least about 40 μm. In some embodiments, the composition comprises at least one pore having an average diameter of at least about 50 μm. In some embodiments, the composition comprises at least one pore having an average diameter of at least about 100 μm. In some embodiments, the composition comprises at least one pore having an average diameter of at least about 1,000 μm. For example, in some embodiments, the composition comprises at least one pore having an average diameter of at least about 10 μm.

In some embodiments, the composition comprises a surface roughness of less than 1,000 μm. In some embodiments, the composition comprises a surface roughness of less than 100 μm. In some embodiments, the composition comprises a surface roughness of less than 10 μm. In some embodiments, the composition comprises a surface roughness of less than 9 μm. In some embodiments, the composition comprises a surface roughness of less than 5 μm. In some embodiments, the composition comprises a surface roughness of less than 2.5 μm. In some embodiments, the composition comprises a surface roughness of less than 1 μm. In some embodiments, the composition comprises a surface roughness of less than 0.9 μm. In some embodiments, the composition comprises a surface roughness of less than 0.8 μm. In some embodiments, the composition comprises a surface roughness of less than 0.7 μm. In some embodiments, the composition comprises a surface roughness of less than 0.6 μm. In some embodiments, the composition comprises a surface roughness of less than 0.5 μm. In some embodiments, the composition comprises a surface roughness of less than 0.4 μm. In some embodiments, the composition comprises a surface roughness of less than 0.3 μm. In some embodiments, the composition comprises a surface roughness of less than 0.2 μm. In some embodiments, the composition comprises a surface roughness of less than 0.1 μm. In some embodiments, the composition comprises a surface roughness of less than 0.09 μm. In some embodiments, the composition comprises a surface roughness of less than 0.08 μm. In some embodiments, the composition comprises a surface roughness of less than 0.05 μm. In some embodiments, the composition comprises a surface roughness of less than 0.02 μm. In some embodiments, the composition comprises a surface roughness of less than 0.01 μm. In some embodiments, the composition comprises a surface roughness of less than 0.001 μm. For example, in some embodiments, the composition comprises a surface roughness of less than 0.2 μm.

In some embodiments, the composition has curing time of less than about 1 year. In some embodiments, the composition has curing time of less than about 1 month. In some embodiments, the composition has curing time of less than about 1 week. In some embodiments, the composition has curing time of less than about 72 h. In some embodiments, the composition has curing time of less than about 48 h. In some embodiments, the composition has curing time of less than about 24 h. In some embodiments, the composition has curing time of less than about 12 h. In some embodiments, the composition has curing time of less than about 6 h. In some embodiments, the composition has curing time of less than about 2 h. In some embodiments, the composition has curing time of less than about 1 h. In some embodiments, the composition has curing time of less than about 30 min. In some embodiments, the composition has curing time of less than about 15 min. In some embodiments, the composition has curing time of less than about 10 min. In some embodiments, the composition has curing time of less than about 5 min. In some embodiments, the composition has curing time of less than about 180 seconds. In some embodiments, the composition has curing time of less than about 120 seconds. In some embodiments, the composition has curing time of less than about 60 seconds. In some embodiments, the composition has curing time of less than about 30 seconds. In some embodiments, the composition has curing time of less than about 20 seconds. In some embodiments, the composition has curing time of less than about 15 seconds. In some embodiments, the composition has curing time of less than about 10 seconds. In some embodiments, the composition has curing time of less than about 5 seconds. For example, in some embodiments, the composition has curing time of between about 50 seconds to about 180 seconds. In some embodiments, the composition has curing time of between about 50 seconds to about 120 seconds. in some embodiments, the composition has curing time of between about 15 seconds to about 20 seconds.

In some embodiments, the composition has a degradation rate of at least about 1% over about 1 year. In some embodiments, the composition has a degradation rate of at least about 1% over about 1 month. In some embodiments, the composition has a degradation rate of at least about 1% over about 14 days. In some embodiments, the composition has a degradation rate of at least about 2% over about 14 days. In some embodiments, the composition has a degradation rate of at least about 5% over about 14 days. In some embodiments, the composition has a degradation rate of at least about 10% over about 14 days. In some embodiments, the composition has a degradation rate of at least about 15% over about 14 days. In some embodiments, the composition has a degradation rate of at least about 20% over about 14 days. In some embodiments, the composition has a degradation rate of at least about 25% over about 14 days. In some embodiments, the composition has a degradation rate of at least about 30% over about 14 days. In some embodiments, the composition has a degradation rate of at least about 35% over about 14 days. In some embodiments, the composition has a degradation rate of at least about 40% over about 14 days. In some embodiments, the composition has a degradation rate of at least about 50% over about 14 days. In some embodiments, the composition has a degradation rate of at least about 60% over about 14 days. In some embodiments, the composition has a degradation rate of at least about 70% over about 14 days. In some embodiments, the composition has a degradation rate of at least about 80% over about 14 days. In some embodiments, the composition has a degradation rate of at least about 90% over about 14 days. In some embodiments, the composition has a degradation rate of at least about 100% over about 14 days. For example, in some embodiments, the composition has a degradation rate of at least about 5% over about 14 days. In some embodiments, the composition has a degradation rate of at least about 40% over about 14 days. In some embodiments, the composition has a degradation rate of at least about 50% over about 14 days. In some embodiments, the composition has a degradation rate of at least about 80% over about 14 days.

In some embodiments, the composition further comprises a nanoparticle, initiator (e.g., photoinitiator, thermal initiator, radical initiator, etc.), stabilizer, polymer, light and/or thermal emitting component, therapeutic agent, binding agent, linking molecule, or any combination thereof.

In one embodiment, the composition further comprises a nanoparticle. In one embodiment, the nanoparticle is a filler. In some embodiments, the nanoparticle is any type of nanoparticle, including, but not limited to, nanocapsules, nanocarrier, nanoclusters, nanovesicles, micelles, block copolymer micelles, lamellae shaped particles, polymersomes, dendrimers, polymer vesicle, and nano-size particles of various other small fabrications that are known to those in the art.

In some embodiments, the nanoparticle is a silicon-containing nanoparticle (e.g., silicon oxide nanoparticle, silanized nanoparticle, or a combination thereof). In some embodiments, the nanoparticle is a silicon-free nanoparticle.

In one embodiment, the nanoparticle is a ceramic nanoparticle. In one embodiment, the nanoparticle comprises a piezoelectric material. For example, in one embodiment, the nanoparticle comprises BaTiO₃ (BTO) nanoparticle.

In some embodiments, the composition comprises at least about 0.01 wt % of the nanoparticle. In some embodiments, the composition comprises at least about 0.02 wt % of the nanoparticle. In some embodiments, the composition comprises at least about 0.03 wt % of the nanoparticle. In some embodiments, the composition comprises at least about 0.04 wt % of the nanoparticle. In some embodiments, the composition comprises at least about 0.05 wt % of the nanoparticle. In some embodiments, the composition comprises at least about 0.06 wt % of the nanoparticle. In some embodiments, the composition comprises at least about 0.07 wt % of the nanoparticle. In some embodiments, the composition comprises at least about 0.08 wt % of the nanoparticle. In some embodiments, the composition comprises at least about 0.09 wt % of the nanoparticle. In some embodiments, the composition comprises at least about 0.1 wt % of the nanoparticle. In some embodiments, the composition comprises at least about 0.2 wt % of the nanoparticle. In some embodiments, the composition comprises at least about 0.25 wt % of the nanoparticle. In some embodiments, the composition comprises at least about 0.5 wt % of the nanoparticle. In some embodiments, the composition comprises at least about 1 wt % of the nanoparticle. In some embodiments, the composition comprises at least about 2 wt % of the nanoparticle. In some embodiments, the composition comprises at least about 3 wt % of the nanoparticle. In some embodiments, the composition comprises at least about 4 wt % of the nanoparticle. In some embodiments, the composition comprises at least about 5 wt % of the nanoparticle. In some embodiments, the composition comprises at least about 9 wt % of the nanoparticle. In some embodiments, the composition comprises at least about 10 wt % of the nanoparticle. In some embodiments, the composition comprises at least about 20 wt % of the nanoparticle. In some embodiments, the composition comprises at least about 30 wt % of the nanoparticle. In some embodiments, the composition comprises at least about 40 wt % of the nanoparticle. In some embodiments, the composition comprises at least about 50 wt % of the nanoparticle. In some embodiments, the composition comprises at least about 60 wt % of the nanoparticle. In some embodiments, the composition comprises at least about 70 wt % of the nanoparticle. In some embodiments, the composition comprises at least about 80 wt % of the nanoparticle. In some embodiments, the composition comprises at least about 90 wt % of the nanoparticle. In some embodiments, the composition comprises at least about 99.99 wt % of the nanoparticle.

In some embodiments, the composition comprises at least about 0.01% w/v of the nanoparticle. In some embodiments, the composition comprises at least about 0.02% w/v of the nanoparticle. In some embodiments, the composition comprises at least about 0.03% w/v of the nanoparticle. In some embodiments, the composition comprises at least about 0.04% w/v of the nanoparticle. In some embodiments, the composition comprises at least about 0.05% w/v of the nanoparticle. In some embodiments, the composition comprises at least about 0.06% w/v of the nanoparticle. In some embodiments, the composition comprises at least about 0.07% w/v of the nanoparticle. In some embodiments, the composition comprises at least about 0.08% w/v of the nanoparticle. In some embodiments, the composition comprises at least about 0.09% w/v of the nanoparticle. In some embodiments, the composition comprises at least about 0.1% w/v of the nanoparticle. In some embodiments, the composition comprises at least about 0.2% w/v of the nanoparticle. In some embodiments, the composition comprises at least about 0.25% w/v of the nanoparticle. In some embodiments, the composition comprises at least about 0.5% w/v of the nanoparticle. In some embodiments, the composition comprises at least about 1% w/v of the nanoparticle. In some embodiments, the composition comprises at least about 2% w/v of the nanoparticle. In some embodiments, the composition comprises at least about 3% w/v of the nanoparticle. In some embodiments, the composition comprises at least about 4% w/v of the nanoparticle. In some embodiments, the composition comprises at least about 5% w/v of the nanoparticle. In some embodiments, the composition comprises at least about 9% w/v of the nanoparticle. In some embodiments, the composition comprises at least about 10% w/v of the nanoparticle. In some embodiments, the composition comprises at least about 20% w/v of the nanoparticle. In some embodiments, the composition comprises at least about 30% w/v of the nanoparticle. In some embodiments, the composition comprises at least about 40% w/v of the nanoparticle. In some embodiments, the composition comprises at least about 50% w/v of the nanoparticle. In some embodiments, the composition comprises at least about 60% w/v of the nanoparticle. In some embodiments, the composition comprises at least about 70% w/v of the nanoparticle. In some embodiments, the composition comprises at least about 80% w/v of the nanoparticle. In some embodiments, the composition comprises at least about 90% w/v of the nanoparticle. In some embodiments, the composition comprises at least about 99.99% w/v of the nanoparticle.

In some embodiments, the composition further comprises an initiator (e.g., photoinitiator, thermal initiator, radical initiator, etc.). In some embodiments, the composition is an initiator-free composition. Examples of such initiators include any initiator known in the art. For example, in some embodiments, the composition further comprises a photoinitiator. Examples of such photoinitiators, thermal initiators, and radical initiators include any photoinitiators, any thermal initiators, and any radical initiators known in the art. For example, in one embodiment, the photoinitiator comprises lithium phenyl-2,4,6-trimethylbenzoyl phosphonate (LAP) or a derivative thereof. In some embodiments, the composition is a photoinitiator-free composition. In another embodiment, the composition is a lithium phenyl-2,4,6-trimethylbenzoyl phosphonate (LAP)-free composition.

In some embodiments, the composition comprises at least about 0.0001 wt % of the initiator (e.g., photoinitiator, thermal initiator, radical initiator, etc.). In some embodiments, the composition comprises at least about 0.001 wt % of the initiator (e.g., photoinitiator, thermal initiator, radical initiator, etc.). In some embodiments, the composition comprises at least about 0.01 wt % of the initiator (e.g., photoinitiator, thermal initiator, radical initiator, etc.). In some embodiments, the composition comprises at least about 0.02 wt % of the initiator (e.g., photoinitiator, thermal initiator, radical initiator, etc.). In some embodiments, the composition comprises at least about 0.03 wt % of the initiator (e.g., photoinitiator, thermal initiator, radical initiator, etc.). In some embodiments, the composition comprises at least about 0.04 wt % of the initiator (e.g., photoinitiator, thermal initiator, radical initiator, etc.). In some embodiments, the composition comprises at least about 0.05 wt % of the initiator (e.g., photoinitiator, thermal initiator, radical initiator, etc.). In some embodiments, the composition comprises at least about 0.06 wt % of the initiator (e.g., photoinitiator, thermal initiator, radical initiator, etc.). In some embodiments, the composition comprises at least about 0.07 wt % of the initiator (e.g., photoinitiator, thermal initiator, radical initiator, etc.). In some embodiments, the composition comprises at least about 0.08 wt % of the initiator (e.g., photoinitiator, thermal initiator, radical initiator, etc.). In some embodiments, the composition comprises at least about 0.09 wt % of the initiator (e.g., photoinitiator, thermal initiator, radical initiator, etc.). In some embodiments, the composition comprises at least about 0.1 wt % of the initiator (e.g., photoinitiator, thermal initiator, radical initiator, etc.). In some embodiments, the composition comprises at least about 0.2 wt % of the initiator (e.g., photoinitiator, thermal initiator, radical initiator, etc.). In some embodiments, the composition comprises at least about 0.25 wt % of the initiator (e.g., photoinitiator, thermal initiator, radical initiator, etc.). In some embodiments, the composition comprises at least about 0.5 wt % of the initiator (e.g., photoinitiator, thermal initiator, radical initiator, etc.). In some embodiments, the composition comprises at least about 1 wt % of the initiator (e.g., photoinitiator, thermal initiator, radical initiator, etc.). In some embodiments, the composition comprises at least about 2 wt % of the initiator (e.g., photoinitiator, thermal initiator, radical initiator, etc.). In some embodiments, the composition comprises at least about 3 wt % of the initiator (e.g., photoinitiator, thermal initiator, radical initiator, etc.). In some embodiments, the composition comprises at least about 4 wt % of the initiator (e.g., photoinitiator, thermal initiator, radical initiator, etc.). In some embodiments, the composition comprises at least about 5 wt % of the initiator (e.g., photoinitiator, thermal initiator, radical initiator, etc.). In some embodiments, the composition comprises at least about 10 wt % of the initiator (e.g., photoinitiator, thermal initiator, radical initiator, etc.). In some embodiments, the composition comprises at least about 20 wt % of the initiator (e.g., photoinitiator, thermal initiator, radical initiator, etc.). In some embodiments, the composition comprises at least about 30 wt % of the initiator (e.g., initiator (e.g., photoinitiator, thermal initiator, radical initiator, etc.), thermal initiator, radical initiator, etc.). In some embodiments, the composition comprises at least about 40 wt % of the initiator (e.g., photoinitiator, thermal initiator, radical initiator, etc.). In some embodiments, the composition comprises at least about 50 wt % of the initiator (e.g., photoinitiator, thermal initiator, radical initiator, etc.). In some embodiments, the composition comprises at least about 60 wt % of the initiator (e.g., photoinitiator, thermal initiator, radical initiator, etc.). In some embodiments, the composition comprises at least about 70 wt % of the initiator (e.g., photoinitiator, thermal initiator, radical initiator, etc.). In some embodiments, the composition comprises at least about 80 wt % of the initiator (e.g., photoinitiator, thermal initiator, radical initiator, etc.). In some embodiments, the composition comprises at least about 90 wt % of the initiator (e.g., photoinitiator, thermal initiator, radical initiator, etc.). In some embodiments, the composition comprises at least about 99.99 wt % of the initiator (e.g., photoinitiator, thermal initiator, radical initiator, etc.).

In some embodiments, the composition comprises at least about 0.0001% w/v of the initiator (e.g., photoinitiator, thermal initiator, radical initiator, etc.). In some embodiments, the composition comprises at least about 0.001% w/v of the initiator (e.g., photoinitiator, thermal initiator, radical initiator, etc.). In some embodiments, the composition comprises at least about 0.01% w/v of the initiator (e.g., photoinitiator, thermal initiator, radical initiator, etc.). In some embodiments, the composition comprises at least about 0.02% w/v of the initiator (e.g., photoinitiator, thermal initiator, radical initiator, etc.). In some embodiments, the composition comprises at least about 0.03% w/v of the initiator (e.g., photoinitiator, thermal initiator, radical initiator, etc.). In some embodiments, the composition comprises at least about 0.04% w/v of the initiator (e.g., photoinitiator, thermal initiator, radical initiator, etc.). In some embodiments, the composition comprises at least about 0.05% w/v of the initiator (e.g., photoinitiator, thermal initiator, radical initiator, etc.). In some embodiments, the composition comprises at least about 0.06% w/v of the initiator (e.g., photoinitiator, thermal initiator, radical initiator, etc.). In some embodiments, the composition comprises at least about 0.07% w/v of the initiator (e.g., photoinitiator, thermal initiator, radical initiator, etc.). In some embodiments, the composition comprises at least about 0.08% w/v of the initiator (e.g., photoinitiator, thermal initiator, radical initiator, etc.). In some embodiments, the composition comprises at least about 0.09% w/v of the initiator (e.g., photoinitiator, thermal initiator, radical initiator, etc.). In some embodiments, the composition comprises at least about 0.1% w/v of the initiator (e.g., photoinitiator, thermal initiator, radical initiator, etc.). In some embodiments, the composition comprises at least about 0.2% w/v of the initiator (e.g., photoinitiator, thermal initiator, radical initiator, etc.). In some embodiments, the composition comprises at least about 0.25% w/v of the initiator (e.g., photoinitiator, thermal initiator, radical initiator, etc.). In some embodiments, the composition comprises at least about 0.5% w/v of the initiator (e.g., photoinitiator, thermal initiator, radical initiator, etc.). In some embodiments, the composition comprises at least about 1% w/v of the initiator (e.g., photoinitiator, thermal initiator, radical initiator, etc.). In some embodiments, the composition comprises at least about 2% w/v of the initiator (e.g., photoinitiator, thermal initiator, radical initiator, etc.). In some embodiments, the composition comprises at least about 3% w/v of the initiator (e.g., photoinitiator, thermal initiator, radical initiator, etc.). In some embodiments, the composition comprises at least about 4% w/v of the initiator (e.g., photoinitiator, thermal initiator, radical initiator, etc.). In some embodiments, the composition comprises at least about 5% w/v of the initiator (e.g., photoinitiator, thermal initiator, radical initiator, etc.). In some embodiments, the composition comprises at least about 10% w/v of the initiator (e.g., photoinitiator, thermal initiator, radical initiator, etc.). In some embodiments, the composition comprises at least about 20% w/v of the initiator (e.g., photoinitiator, thermal initiator, radical initiator, etc.). In some embodiments, the composition comprises at least about 30% w/v of the initiator (e.g., photoinitiator, thermal initiator, radical initiator, etc.). In some embodiments, the composition comprises at least about 40% w/v of the initiator (e.g., photoinitiator, thermal initiator, radical initiator, etc.). In some embodiments, the composition comprises at least about 50% w/v of the initiator (e.g., photoinitiator, thermal initiator, radical initiator, etc.). In some embodiments, the composition comprises at least about 60% w/v of the initiator (e.g., photoinitiator, thermal initiator, radical initiator, etc.). In some embodiments, the composition comprises at least about 70% w/v of the initiator (e.g., photoinitiator, thermal initiator, radical initiator, etc.). In some embodiments, the composition comprises at least about 80% w/v of the initiator (e.g., photoinitiator, thermal initiator, radical initiator, etc.). In some embodiments, the composition comprises at least about 90% w/v of the initiator (e.g., photoinitiator, thermal initiator, radical initiator, etc.). In some embodiments, the composition comprises at least about 99.99% w/v of the initiator (e.g., photoinitiator, thermal initiator, radical initiator, etc.).

In some embodiments, the composition is a light emitting component-free composition, thermal emitting component-free composition, or a combination thereof. In some embodiments, the composition further comprises a light emitting component, thermal emitting component, or a combination thereof. Examples of such light emitting components and thermal emitting components include any light emitting component and any thermal emitting component known in the art. For example, in one embodiment, the light emitting component comprises optical fiber. In another embodiment, the composition is an optical fiber-free composition.

In some embodiments, the composition comprises at least about 0.0001 wt % of the light emitting component, thermal emitting component, or a combination thereof. In some embodiments, the composition comprises at least about 0.001 wt % of the light emitting component, thermal emitting component, or a combination thereof. In some embodiments, the composition comprises at least about 0.01 wt % of the light emitting component, thermal emitting component, or a combination thereof. In some embodiments, the composition comprises at least about 0.02 wt % of the light emitting component, thermal emitting component, or a combination thereof. In some embodiments, the composition comprises at least about 0.03 wt % of the light emitting component, thermal emitting component, or a combination thereof. In some embodiments, the composition comprises at least about 0.04 wt % of the light emitting component, thermal emitting component, or a combination thereof. In some embodiments, the composition comprises at least about 0.05 wt % of the light emitting component, thermal emitting component, or a combination thereof. In some embodiments, the composition comprises at least about 0.06 wt % of the light emitting component, thermal emitting component, or a combination thereof. In some embodiments, the composition comprises at least about 0.07 wt % of the light emitting component, thermal emitting component, or a combination thereof. In some embodiments, the composition comprises at least about 0.08 wt % of the light emitting component, thermal emitting component, or a combination thereof. In some embodiments, the composition comprises at least about 0.09 wt % of the light emitting component, thermal emitting component, or a combination thereof. In some embodiments, the composition comprises at least about 0.1 wt % of the light emitting component, thermal emitting component, or a combination thereof. In some embodiments, the composition comprises at least about 0.2 wt % of the light emitting component, thermal emitting component, or a combination thereof. In some embodiments, the composition comprises at least about 0.25 wt % of the light emitting component, thermal emitting component, or a combination thereof. In some embodiments, the composition comprises at least about 0.5 wt % of the light emitting component, thermal emitting component, or a combination thereof. In some embodiments, the composition comprises at least about 1 wt % of the light emitting component, thermal emitting component, or a combination thereof. In some embodiments, the composition comprises at least about 2 wt % of the light emitting component, thermal emitting component, or a combination thereof. In some embodiments, the composition comprises at least about 3 wt % of the light emitting component, thermal emitting component, or a combination thereof. In some embodiments, the composition comprises at least about 4 wt % of the light emitting component, thermal emitting component, or a combination thereof. In some embodiments, the composition comprises at least about 5 wt % of the light emitting component, thermal emitting component, or a combination thereof. In some embodiments, the composition comprises at least about 10 wt % of the light emitting component, thermal emitting component, or a combination thereof. In some embodiments, the composition comprises at least about 20 wt % of the light emitting component, thermal emitting component, or a combination thereof. In some embodiments, the composition comprises at least about 30 wt % of the light emitting component, thermal emitting component, or a combination thereof. In some embodiments, the composition comprises at least about 40 wt % of the light emitting component, thermal emitting component, or a combination thereof. In some embodiments, the composition comprises at least about 50 wt % of the light emitting component, thermal emitting component, or a combination thereof. In some embodiments, the composition comprises at least about 60 wt % of the light emitting component, thermal emitting component, or a combination thereof. In some embodiments, the composition comprises at least about 70 wt % of the light emitting component, thermal emitting component, or a combination thereof. In some embodiments, the composition comprises at least about 80 wt % of the light emitting component, thermal emitting component, or a combination thereof. In some embodiments, the composition comprises at least about 90 wt % of the light emitting component, thermal emitting component, or a combination thereof. In some embodiments, the composition comprises at least about 99.99 wt % of the light emitting component, thermal emitting component, or a combination thereof.

In some embodiments, the composition further comprises a stabilizer. In some embodiments, the composition is a stabilizer-free composition. In some embodiments, the stabilizer is a primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

In some embodiments, the composition comprises at least about 0.0001 wt % of the stabilizer. In some embodiments, the composition comprises at least about 0.001 wt % of the stabilizer. In some embodiments, the composition comprises at least about 0.01 wt % of the stabilizer. In some embodiments, the composition comprises at least about 0.02 wt % of the stabilizer. In some embodiments, the composition comprises at least about 0.03 wt % of the stabilizer. In some embodiments, the composition comprises at least about 0.04 wt % of the stabilizer. In some embodiments, the composition comprises at least about 0.05 wt % of the stabilizer. In some embodiments, the composition comprises at least about 0.06 wt % of the stabilizer. In some embodiments, the composition comprises at least about 0.07 wt % of the stabilizer. In some embodiments, the composition comprises at least about 0.08 wt % of the stabilizer. In some embodiments, the composition comprises at least about 0.09 wt % of the stabilizer. In some embodiments, the composition comprises at least about 0.1 wt % of the stabilizer. In some embodiments, the composition comprises at least about 0.2 wt % of the stabilizer. In some embodiments, the composition comprises at least about 0.25 wt % of the stabilizer. In some embodiments, the composition comprises at least about 0.5 wt % of the stabilizer. In some embodiments, the composition comprises at least about 1 wt % of the stabilizer. In some embodiments, the composition comprises at least about 2 wt % of the stabilizer. In some embodiments, the composition comprises at least about 3 wt % of the stabilizer. In some embodiments, the composition comprises at least about 4 wt % of the stabilizer. In some embodiments, the composition comprises at least about 5 wt % of the stabilizer. In some embodiments, the composition comprises at least about 10 wt % of the stabilizer. In some embodiments, the composition comprises at least about 20 wt % of the stabilizer. In some embodiments, the composition comprises at least about 30 wt % of the stabilizer. In some embodiments, the composition comprises at least about 40 wt % of the stabilizer. In some embodiments, the composition comprises at least about 50 wt % of the stabilizer. In some embodiments, the composition comprises at least about 60 wt % of the stabilizer. In some embodiments, the composition comprises at least about 70 wt % of the stabilizer. In some embodiments, the composition comprises at least about 80 wt % of the stabilizer. In some embodiments, the composition comprises at least about 90 wt % of the stabilizer. In some embodiments, the composition comprises at least about 99.99 wt % of the stabilizer.

In one embodiment, the composition further comprises a polymer. In some embodiments, the polymer may be a straight chain polymer (i.e., linear polymer) or a branched chain polymer (i.e., branched polymer), including hyperbranched polymers. In one embodiment the polymer is a branched polymer. In one embodiment, the polymer is cross-linked.

In some embodiments, the polymer comprises a homopolymer, copolymer, block copolymer, or any combination thereof. In some embodiments, the block copolymer is a triblock, tetrablock, pentablock, or at least six block copolymer.

In one embodiment, the polymer has molecular weight of about 1 kDa to about 10,000 kDa. For example, in one embodiment, the polymer has a molecular weight of about 5 kDa to about 2,000 kDa, about 5 kDa to about 1,500 kDa, about 5 kDa to about 1,000 kDa, about 5 kDa to about 800 kDa, about 5 kDa to about 500 kDa, about 5 kDa to about 300 kDa or about 5 kDa to about 200 kDa or about 800 kDa to about 3,000 kDa.

In some embodiments, the polymer is a polyacrylate, polymethacrylate, polyamine, polyalkyleneimine (e.g., polyethyleneimine), polyallylamine, polyamidoamine, polyolefin, poly(amino-co-ester), or any combination thereof. Examples of polymers include, but are not limited to, polymethyl methacrylate (PMMA), poly bisphenol A diglycidyl dimethacrylate, poly triethylene glycol dimethacrylate, polyethyleneimine (PEI), chitosan, poly(2-N,N-dimethylaminoethylmethacrylate), poly-L-lysine, poly(ethylene oxide) (PEO) block copolymer, poly(ethylethylene) (PEE), poly(butadiene) (PB or PBD), poly(styrene) (PS), poly(isoprene) (PI), poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-gluatamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, poly(F-caprolactone) (PCL) diblock co-polymer, poly(ethylene oxide)-block-poly(F-caprolactone) (PEO-b-PCL) based diblock copolymers, poly(lactic acid), poly(glycolide), poly(lactic-coglycolic acid), poly(3-hydroxybutyrate), or any combination thereof.

In some embodiments, the composition comprises at least about 0.0001 wt % of the polymer. In some embodiments, the composition comprises at least about 0.001 wt % of the polymer. In some embodiments, the composition comprises at least about 0.01 wt % of the polymer. In some embodiments, the composition comprises at least about 0.02 wt % of the polymer. In some embodiments, the composition comprises at least about 0.03 wt % of the polymer. In some embodiments, the composition comprises at least about 0.04 wt % of the polymer. In some embodiments, the composition comprises at least about 0.05 wt % of the polymer. In some embodiments, the composition comprises at least about 0.06 wt % of the polymer. In some embodiments, the composition comprises at least about 0.07 wt % of the polymer. In some embodiments, the composition comprises at least about 0.08 wt % of the polymer. In some embodiments, the composition comprises at least about 0.09 wt % of the polymer. In some embodiments, the composition comprises at least about 0.1 wt % of the polymer. In some embodiments, the composition comprises at least about 0.2 wt % of the polymer. In some embodiments, the composition comprises at least about 0.25 wt % of the polymer. In some embodiments, the composition comprises at least about 0.5 wt % of the polymer. In some embodiments, the composition comprises at least about 1 wt % of the polymer. In some embodiments, the composition comprises at least about 2 wt % of the polymer. In some embodiments, the composition comprises at least about 3 wt % of the polymer. In some embodiments, the composition comprises at least about 4 wt % of the polymer. In some embodiments, the composition comprises at least about 5 wt % of the polymer. In some embodiments, the composition comprises at least about 10 wt % of the polymer. In some embodiments, the composition comprises at least about 20 wt % of the polymer. In some embodiments, the composition comprises at least about 30 wt % of the polymer. In some embodiments, the composition comprises at least about 40 wt % of the polymer. In some embodiments, the composition comprises at least about 50 wt % of the polymer. In some embodiments, the composition comprises at least about 60 wt % of the polymer. In some embodiments, the composition comprises at least about 70 wt % of the polymer. In some embodiments, the composition comprises at least about 80 wt % of the polymer. In some embodiments, the composition comprises at least about 90 wt % of the polymer. In some embodiments, the composition comprises at least about 99.99 wt % of the polymer.

In one aspect of the invention, the composition further comprises a therapeutic agent. In some embodiments, the composition further comprises a therapeutically effective amount of the therapeutic agent. In one embodiment, the nanoparticle comprises the therapeutic agent. In one embodiment, the therapeutic agent is a hydrophobic therapeutic agent. In one embodiment, the therapeutic agent is a hydrophilic therapeutic agent. Examples of such therapeutic agents include, but are not limited to, one or more drugs, proteins, amino acids, peptides, antibodies, antibiotics, anti-inflammatory agents, anti-infection agents, anti-bacterial agents, anti-viral agents, anti-fungal agents, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In one embodiment, the therapeutic agent may be an anti-infection agent. Any suitable anti-infection agent may be used in the compositions and methods of the present disclosure. The selection of a suitable anti-infection agent may depend upon, among other things, the type of infection to be treated and the composition of the present disclosure. In certain embodiments, the anti-infection agent may be an anti-bacterial agent, anti-fungal agent, anti-viral agent, or any combination thereof. Thus, in certain embodiments, the anti-infection agent may be effective for treating one or more of bacterial infection, viral infection, fungal infection, or any combination thereof.

Examples of antibacterial agents or antibiotics include, but are not limited to, aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

Additional nonlimiting examples of antibacterial agents include Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmnenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Gatifloxacin Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarnoxicillin; Sarpicillin; Scopafingin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Suifabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

Examples of anti-fungal agent include, but are not limited to, polyenes (e.g., amphotericin b, candicidin, mepartricin, natamycin, and nystatin), allylamines (e.g., butenafine, and naftifine), imidazoles (e.g., bifonazole, butoconazole, chlordantoin, flutrimazole, isoconazole, ketoconazole, and lanoconazole), thiocarbamates (e.g., tolciclate, tolindate, and tolnaftate), triazoles (e.g., fluconazole, itraconazole, saperconazole, and terconazole), bromosalicylchloranilide, buclosamide, calcium propionate, chlorphenesin, ciclopirox, azaserine, griseofulvin, oligomycins, neomycin undecylenate, pyrrolnitrin, siccanin, tubercidin, and viridin. Additional examples of antifungal compounds include but are not limited to Acrisorcin; Ambruticin; Amphotericin B; Azaconazole; Azaserine; Basifungin; Bifonazole; Biphenamine Hydrochloride; Bispyrithione Magsulfex; Butoconazole Nitrate; Calcium Undecylenate; Candicidin; Carbol-Fuchsin; Chlordantoin; Ciclopirox; Ciclopirox Olamine; Cilofungin; Cisconazole; Clotrimazole; Cuprimyxin; Denofungin; Dipyrithione; Doconazole; Econazole; Econazole Nitrate; Enilconazole; Ethonam Nitrate; Fenticonazole Nitrate; Filipin; Fluconazole; Flucytosine; Fungimycin; Griseofulvin; Hamycin; Isoconazole; Itraconazole; Kalafungin; Ketoconazole; Lomofingin; Lydimycin; Mepartricin; Miconazole; Miconazole Nitrate; Monensin; Monensin Sodium; Naftifine Hydrochloride; Neomycin Undecylenate; Nifuratel; Nifurmerone; Nitralamine Hydrochloride; Nystatin; Octanoic Acid; Orconazole Nitrate; Oxiconazole Nitrate; Oxifungin Hydrochloride; Parconazole Hydrochloride; Partricin; Potassium Iodide; Proclonol; Pyrithione Zinc; Pyrrolnitrin; Rutamycin; Sanguinarium Chloride; Saperconazole; Scopafungin; Selenium Sulfide; Sinefungin; Sulconazole Nitrate; Terbinafine; Terconazole; Thiram; Ticlatone; Tioconazole; Tolciclate; Tolindate; Tolnaftate; Triacetin; Triafuigin; Undecylenic Acid; Viridoflilvin; Zinc Undecylenate; and Zinoconazole Hydrochloride.

Examples of anti-viral agents include, but are not limited to, proteins, polypeptides, peptides, fusion protein antibodies, nucleic acid molecules, organic molecules, inorganic molecules, and small molecules that inhibit or reduce the attachment of a virus to its receptor, the internalization of a virus into a cell, the replication of a virus, or release of virus from a cell. Many examples of antiviral compounds that can be used in combination with the compounds of the invention are known in the art and include but are not limited to: rifampicin, nucleoside reverse transcriptase inhibitors (e.g., AZT, ddl, ddC, 3TC, d4T), non-nucleoside reverse transcriptase inhibitors (e.g., Efavirenz, Nevirapine), protease inhibitors (e.g., aprenavir, indinavir, ritonavir, and saquinavir), idoxuridine, cidofovir, acyclovir, ganciclovir, zanamivir, amantadine, and Palivizumab. Other examples of anti-viral agents include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime, zinc, heparin, anionic polymers.

In one embodiment, the therapeutic agent may be an anti-inflammatory agent. Any suitable anti-inflammatory agent may be used in the compositions and methods of the present disclosure. The selection of a suitable anti-inflammatory agent may depend upon, among other things, the type of inflammation to be treated and the composition of the present disclosure. Examples of anti-inflammatory agents include, but are not limited to, non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib, diclofenac, etodolac, fenoprofen, indomethacin, ketoralac, oxaprozin, nabumentone, sulindac, tolmentin, rofecoxib, naproxen, ketoprofen and nabumetone. Such NSAIDs function by inhibiting a cyclooxgenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as prostaglandins, thromboxanes, and leukotrienes.

In one embodiment, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the composition of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, in one embodiment, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In one embodiment, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In one embodiment, the therapeutic agent is one or more non-therapeutic moieties. In some embodiments, the composition comprises one or more therapeutic moieties, one or more non-therapeutic moieties, or any combination thereof.

In some embodiments, the composition comprises therapeutic agent with controlled release that can be “triggered” by one of the following stimulus: polymer degradation, enzyme sensitive polymers, or by an external stimulus, such as electrical charge, acoustic waves, mastication load, pH change, ultrasound, light, UV light, NIR light. Thus, in some embodiments, the compositions of the present disclosure may be “remotely triggered” by applying stimulus (e.g., energy, such as electrical charge, acoustic waves, mastication load) to the composition. In some embodiments, the energy, such as an electrical charge, acoustic waves, mastication load, electromagnetic field, magnetic field, optical methods (e.g., ultraviolet (UV) irradiation, UV-vis-NIR irradiation, infrared (IR) irradiation, NIR irradiation), or specific radiofrequencies, may be applied to the composition thereby causing the release of a therapeutic agent from the nanoparticle further comprising the therapeutic agent. For example, in one embodiment, an electrical charge is applied to a composition comprising a therapeutic agent, wherein the electrical charge causes the release of the therapeutic agent from the composition. In another embodiment, an acoustic wave is applied to a composition comprising a therapeutic agent, wherein the acoustic waves cause the release of the therapeutic agent from the composition. In another embodiment, a mastication load is applied to a composition comprising a therapeutic agent, wherein the mastication load causes the release of the therapeutic agent from the composition. In some embodiments, this may provide a clinician the ability to control and visualize drug therapy noninvasively.

In some embodiments, the composition further comprises a bonding agent and/or linking molecule. Examples of linking molecules include, but are not limited to, bisphenol A diglycidyl dimethacrylate, triethylene glycol dimethacrylate, camphorquinone, ethyl 4-dimethylaminobenzoate, poly(ethylene glycol) and its derivatives, azide compounds, maleimide compounds, hydzrazine compounds, dibenzo-cyclooctyne (DBCO) compounds, dithiol compounds, dithiol compounds with hydrazide and/or carboxylic functionality, or single thiols and/or amines or their derivatives.

In some embodiments, the composition comprises at least about 0.0001 wt % of the bonding agent and/or linking molecule. In some embodiments, the composition comprises at least about 0.001 wt % of the bonding agent and/or linking molecule. In some embodiments, the composition comprises at least about 0.01 wt % of the bonding agent and/or linking molecule. In some embodiments, the composition comprises at least about 0.02 wt % of the bonding agent and/or linking molecule. In some embodiments, the composition comprises at least about 0.03 wt % of the bonding agent and/or linking molecule. In some embodiments, the composition comprises at least about 0.04 wt % of the bonding agent and/or linking molecule. In some embodiments, the composition comprises at least about 0.05 wt % of the bonding agent and/or linking molecule. In some embodiments, the composition comprises at least about 0.06 wt % of the bonding agent and/or linking molecule. In some embodiments, the composition comprises at least about 0.07 wt % of the bonding agent and/or linking molecule. In some embodiments, the composition comprises at least about 0.08 wt % of the bonding agent and/or linking molecule. In some embodiments, the composition comprises at least about 0.09 wt % of the bonding agent and/or linking molecule. In some embodiments, the composition comprises at least about 0.1 wt % of the bonding agent and/or linking molecule. In some embodiments, the composition comprises at least about 0.2 wt % of the bonding agent and/or linking molecule. In some embodiments, the composition comprises at least about 0.25 wt % of the bonding agent and/or linking molecule. In some embodiments, the composition comprises at least about 0.5 wt % of the bonding agent and/or linking molecule. In some embodiments, the composition comprises at least about 1 wt % of the bonding agent and/or linking molecule. In some embodiments, the composition comprises at least about 2 wt % of the bonding agent and/or linking molecule. In some embodiments, the composition comprises at least about 3 wt % of the bonding agent and/or linking molecule. In some embodiments, the composition comprises at least about 4 wt % of the bonding agent and/or linking molecule. In some embodiments, the composition comprises at least about 5 wt % of the bonding agent and/or linking molecule. In some embodiments, the composition comprises at least about 10 wt % of the bonding agent and/or linking molecule. In some embodiments, the composition comprises at least about 20 wt % of the bonding agent and/or linking molecule. In some embodiments, the composition comprises at least about 30 wt % of the bonding agent and/or linking molecule. In some embodiments, the composition comprises at least about 40 wt % of the bonding agent and/or linking molecule. In some embodiments, the composition comprises at least about 50 wt % of the bonding agent and/or linking molecule. In some embodiments, the composition comprises at least about 60 wt % of the bonding agent and/or linking molecule. In some embodiments, the composition comprises at least about 70 wt % of the bonding agent and/or linking molecule. In some embodiments, the composition comprises at least about 80 wt % of the bonding agent and/or linking molecule. In some embodiments, the composition comprises at least about 90 wt % of the bonding agent and/or linking molecule. In some embodiments, the composition comprises at least about 99.99 wt % of the bonding agent and/or linking molecule.

In some embodiments, the composition may optionally further comprise a biocompatible metal. Examples of biocompatible metals include, but are not limited to, titanium, copper, copper sulfide, iron oxide, cobalt and noble metals, such as gold and/or silver. One of ordinary skill in the art will be able to select of a suitable type of nanoparticle taking into consideration at least the type of imaging and/or therapy to be performed.

In some embodiments, the composition is a biocompatible composition, biodegradable composition, antimicrobial composition, or any combination thereof.

In one aspect of the invention, the composition is a biomaterial. In one embodiment, the biomaterial comprises a nanoparticle comprising a piezoelectric material.

In one embodiment, the biomaterial is a medical device. In one embodiment, the medical device is an implant. In one embodiment, the medical device is a catheter. In one embodiment, the medical device is a dental composition. In one embodiment, the biomaterial is a coating. In one embodiment, the biomaterial is a surface coating. In one embodiment, the biomaterial is a surface coating of medical devices. For example, in one embodiment, the biomaterial is a surface coating of titanium implant.

In one embodiment, the composition promotes bone growth, prevents, reduces, or treats bone damage and/or bone structure damage, prevents, reduces, or treats tooth damage and/or tooth structure damage, prevents, reduces, or treats an infection, prevents, reduces, or treats inflammation (e.g., gum inflammation, gingivitis, etc.), prevents or reduces biofilm formation, prevents or treats diseases, disorders, or conditions (e.g., oral diseases, disorders, or conditions, such as periodontal disease, peri-implantitis, oral infection, oral cancer, or any combination thereof), or any combination thereof. In some embodiments, the infection is a bacterial infection, fungal infection, viral infection, or any combination thereof. In some embodiments, the composition improves oral health and prevents oral cancer. For example, in some embodiments, the piezoelectric material promotes bone growth, prevents, reduces, or treats bone damage and/or bone structure damage, prevents, reduces, or treats tooth damage and/or tooth structure damage, prevents, reduces, or treats an infection, prevents, reduces, or treats inflammation (e.g., gum inflammation, gingivitis, etc.), or any combination thereof.

In various aspects, the present invention also provides a pharmaceutical composition comprising at least one composition of the present invention. In some embodiments, the pharmaceutical composition further comprises a pharmaceutically acceptable carrier.

### Methods

The present invention also provides a method of treating biofilms, preventing or reducing biofilm formation, promoting mineralization, tooth growth, and/or bone growth, preventing, reducing, or treating bone damage and/or bone structure damage, preventing, reducing, or treating tooth damage and/or tooth structure damage, promoting antimicrobial effect, promoting antiinfection effect (e.g., promoting antibacterial effect, promoting antifungal effect, promoting antiviral effect, etc.), preventing, reducing, or treating an infection, promoting anti-inflammatory effect, preventing, reducing, or treating inflammation (e.g., gum inflammation, gingivitis, etc.), preventing, reducing, or treating diseases, disorders, or conditions (e.g., oral diseases, disorders, or conditions, such as periodontal disease, peri-implantitis, oral infection, oral cancer, or any combination thereof), improving health of a subject in need thereof, or any combination thereof in a subject in need thereof.

In one aspect, the present invention provides a method of preventing, reducing, or treating infection, preventing, reducing, or treating inflammation (e.g., gum inflammation, gingivitis, etc.), or any combination thereof in a subject in need thereof. In one embodiment, the present invention provides a method of preventing, reducing, or treating fungal infection, bacterial infection, viral infection, or any combination thereof in a subject in need thereof. Thus, in some embodiments, the disease, disorder, or condition comprises an infection, inflammation (e.g., gum inflammation, gingivitis, etc.), cancer, or any combination thereof.

In various aspects, the present invention comprises a method of simultaneously treating biofilms, preventing or reducing biofilm formation, promoting mineralization, tooth growth, and/or bone growth, preventing, reducing, or treating bone damage and/or bone structure damage, preventing, reducing, or treating tooth damage and/or tooth structure damage, promoting antimicrobial effect, promoting antiinfection effect (e.g., promoting antibacterial effect, promoting antifungal effect, promoting antiviral effect, etc.), preventing, reducing, or treating an infection, promoting anti-inflammatory effect, preventing, reducing, or treating inflammation (e.g., gum inflammation, gingivitis, etc.), preventing, reducing, or treating diseases, disorders, or conditions (e.g., oral diseases, disorders, or conditions, such as periodontal disease, peri-implantitis, oral infection, oral cancer, or any combination thereof), improving health of a subject in need thereof, or any combination thereof in a subject in need thereof.

In another aspect, the present invention provides a method of administering at least one composition of the present invention to a subject in need thereof. In some embodiments, the subject has a disease, disorder, or condition or is at risk of developing a disease, disorder, or condition. Examples of such diseases, disorders, or conditions include, but are not limited to cancer, oral diseases or disorders, such as periodontal disease, peri-implantitis, oral infection, and/or oral cancer, loss of tooth structure, loss of tooth, reduced or abnormal biofilm formation, reduced or abnormal mineralization, bone damage and/or bone structure damage, bone structure damage, tooth damage, tooth structure damage, infection, inflammation, such as gum inflammation, gingivitis, etc., reduced health, or any combination thereof.

In various embodiments, the method comprises administering at least one composition of the present invention to the subject in need thereof. In one embodiment, the method comprises applying at least one composition of the present invention to the subject in need thereof. For example, in one embodiment, the method comprises applying a composition to at least a portion of a tooth of the subject in need thereof.

In various embodiments, the method comprises a stimulus. In various embodiments, the method comprises a triggering stimulus. In various embodiments, the triggering stimulus repels biofilms, prevents or reduces biofilm formation, promotes mineralization, tooth growth, and/or bone growth, prevents, reduces, or treats bone damage and/or bone structure damage, prevents, reduces, or treats tooth damage and/or tooth structure damage, promotes antimicrobial effect, promotes antiinfection effect (e.g., antibacterial effect, antifungal effect, antiviral effect, etc.), promotes anti-inflammatory effect, or any combination thereof. For example, in one embodiment, the method comprises mastication forces. In some embodiments, the subject generates mastication forces. In one embodiment, the mastication forces generate electrical charges at the composition's surface. In some embodiments, the mastication forces excite the material, which generates electrical charges at the material's surface. In some embodiments, the electrical charges repel biofilms, precipitate new minerals, or any combination thereof.

In another aspect, the present invention provides a method of improving health of a subject in need thereof. For example, in one embodiment, the present invention provides a method of improving oral health of a subject in need thereof. Thus, in one embodiment, the present invention provides a method of improving oral health of a subject in need thereof, which prevents oral cancer.

The following are non-limiting examples of cancers that can be prevented, reduced, or treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; liposarcoma and anaplastic liposarcoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; biliary track cancer, cholangiocarcinoma, anal cancer, neuroendocrine tumors, small bowel cancer, gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; Sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; and Wilms tumor.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the exemplary embodiments of the present invention, and are not to be construed as limiting in any way the remainder of the disclosure.

### Example 1: A Novel Injectable Piezoelectric Hydrogel for Periodontal Disease Treatment

Injectable dental biomaterials have gained popularity for the nonsurgical treatment of PD because of their conformability and facile flowability into the periodontal pocket (Alonso et al., 2021, Polymers (Basel), 13:650; Dong et al., 2019, ACS Appl. Bio Mater., 2:5985-5994; Patrick et al., 2022, Sci. Rep., 12:15811; Ribeiro et al., 2020, ACS Appl. Mater. Interfaces., 12:16006-16017). Specifically, hydrogels are excellent candidates because of their biocompatibility, adhesion, biodegradation, affordability, and simple chemistry (Li et al., 2018, Antibacterial Hydrogels, 5:1700527; Ribeiro et al., 2020, Biomacromolecules, 21:3945-3956). Gelatin methacryloyl (GelMA) is a common biomedical hydrogel that has been incorporated with different antimicrobial agents, including zinc, quaternary ammonium, and antibiotics (e.g., ciprofloxacin) for PD treatment (Vargas-Alfredo et al., 2022, Gels, 8:630; Shirzaei Sani et al., 2019, Matter, 1:926-944; Goto et al., 2021, Int. J. Mol. Sci., 22:1635; Liu et al., 2022, Acta Biomater., 146:37-48; Dubey et al., 2020, Acta Biomater., 113:164-176). Hydrogels enable long-term leaching of the agents with fewer reapplication cycles (Solovieva et al., 2020, J. Photochem. Photobiol. B, 210:111954; Huang et al., 2022, Biomater Sci., 10:6413-6446). However, some limitations still exist, including uncontrolled and rapid drug release, microbial resistance build-up, and a lack of complete periodontal tissue regeneration (Li et al., 2022, Gels, 8:624).

Ideal hydrogels for PD treatment are expected to not only inhibit bacterial activity to control the inflammatory response but also foster tissue regeneration. The use of multifunctional hydrogels loaded with antibacterial, antioxidant, and/or tissue regeneration agents have been proposed recently (Lee et al., 2023, Smart Mater. Med., 4:460-467). The goal of this approach is to deter bacteria, reduce the imbalance in the redox system, prevent bone resorption, and foster tissue regeneration in the periodontium. For example, a gelatin-chitosan thermosensitive hydrogel was loaded with aspirin and erythropoietin to exert anti-inflammation and tissue regeneration effects, respectively (Xu et al., 2019, Acta Biomater., 86:235-246). Periodontal pathogens produce and release chemicals that activate the innate immune system to release proinflammatory cytokines contributing to the progression of the disease (Usui et al., 2021, Japanese Dental Science Review, 57:201-208; Ramadan et al., 2020, Eur. J. Dent., 14:483). To terminate alveolar bone resorption and resolve PD, simultaneous antibacterial and tissue regeneration effects are required. This approach is limited in the existing nonsurgical treatment options for PD.

Piezoelectric materials produce electrical charges in response to external vibrations and mechanical stimulation (Zaszczynska et al., 2020, Polymers (Basel), 12:2754). These materials have been extensively used for energy harvesting devices, sensors, and medical applications (Zaszczynska et al., 2020, Polymers (Basel), 12:2754; Ribeiro et al., 2015, Colloids Surf. B Biointerfaces, 136:46-55). For example, piezoelectric polymers have been used for orthopedic bone tissue regeneration by promoting osteogenic proliferation and differentiation due to their electromechanical properties (Kao et al., 2019, Sci. Technol. Adv. Mater., 20:1103-1117). Barium titanate (BaTiO₃, BTO) is a biocompatible piezoelectric ceramic that has gained substantial interest in recent years due to accepted biocompatibility, high dielectric constant, excellent ferroelectric properties, and the possibility of nanoparticle fabrication (Acosta et al., 2017, Appl. Phys. Rev., 4:041305; Zhang et al., 2014, Materials Science and Engineering C, 39:143-149). These qualities have pushed BTO as a prime candidate for the development of biomedical technologies and wearable bioelectronics.

Piezoelectric materials have recently been used in dentistry for antimicrobial, remineralization, and tissue engineering applications (Ghosh et al., 2023, J. Funct. Biomater., 14:8; Montoya et al., 2021, ACS Appl. Mater. Interfaces., 13:43868-43879). For example, a piezoelectric resin composite was developed after mixing BTO fillers with a standard polymer matrix. This material prevented the formation of carries-related biofilms after showing a 3 log reduction on the bacterial load (Montoya et al., 2021, ACS Appl. Mater. Interfaces., 13:43868-43879). In a different study, BTO fillers were added into PMMA to enable antifungal effects that prevented denture-related pathogenic biofilms (Montoya et al., 2021, ACS Biomater Sci. Eng., 7:4838-4846). These studies attributed the antimicrobial mechanism to reactive oxygen species (ROS) production and oxidative stress. Despite the many advantages of BTO, the antimicrobial effects against periodontal pathogens and periodontal tissue regeneration have not been elucidated.

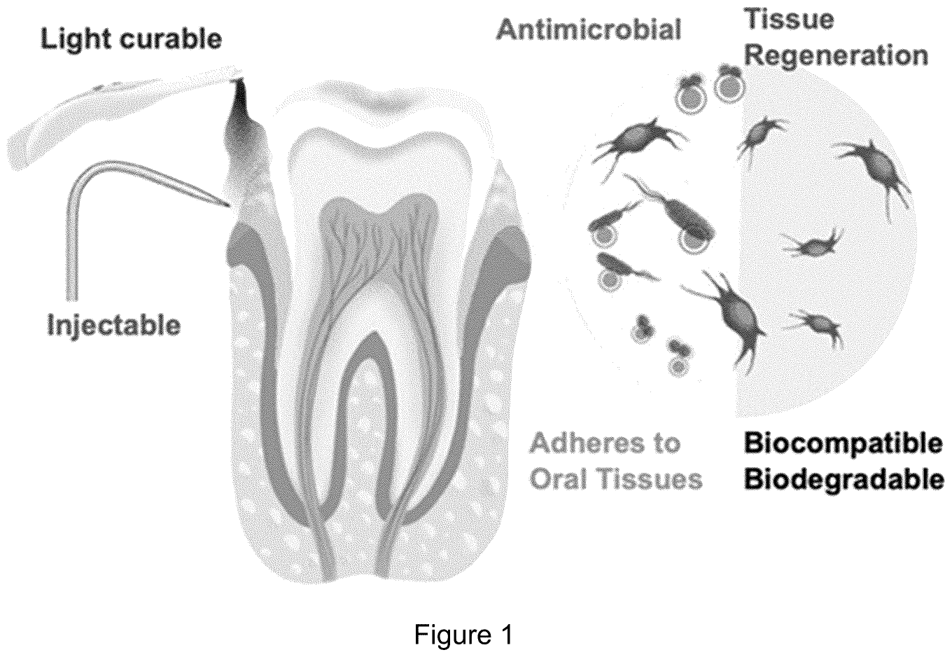

In this study, an injectable multifunctional piezoelectric hydrogel incorporated with BTO fillers (Piezo-GEL) for the nonsurgical treatment of PD was developed (FIG. 1). The new biomaterial offers a unique combination of antibacterial and bone tissue regeneration effects. Using a minimally invasive probe, PiezoGEL can be locally injected into an infected periodontal pocket and light-cured to form a graft in situ. PiezoGEL is a minimally invasive treatment that removes pathogens from the periodontal pocket without using drugs. In addition, the same biomaterial system promotes alveolar bone tissue regeneration in the periodontium. Particularly, in this study, both the antibacterial and bone tissue regeneration effects for periodontal disease treatment were evaluated, both in vitro and in vivo.

### Hydrogels Characterization

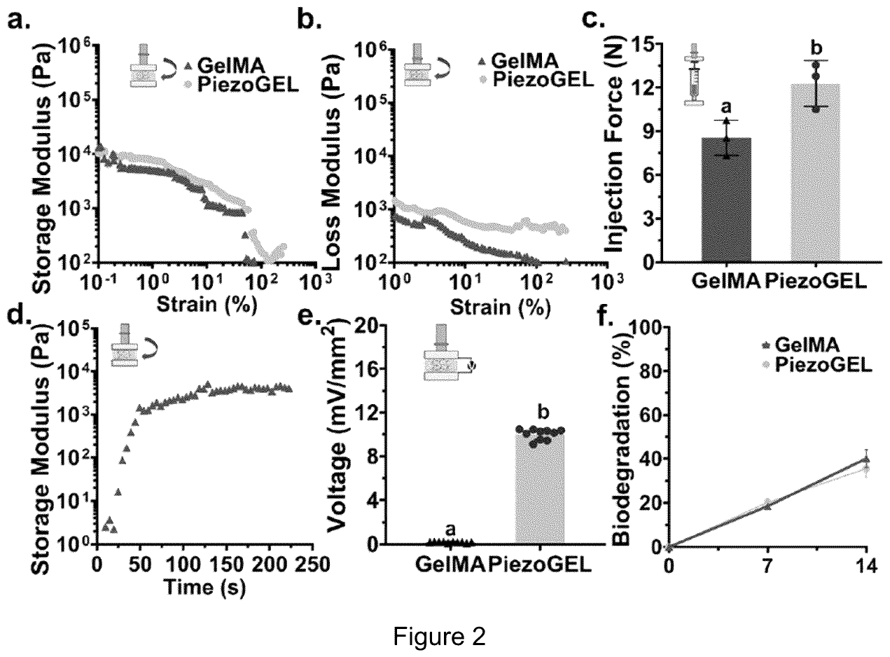

Overall, the rheological evaluations showed that adding BTO to GelMA did not significantly affect the loss and storage modulus (FIG. 2A and FIG. 2B). Shear-thinning behavior was observed for the two formulations (GelMA and PiezoGEL). Such behavior is ideal for clinical application since it facilitates the manipulation and delivery of the hydrogel into the periodontal pocket. A similar storage modulus (10⁴ Pa) was measured for both hydrogel formulations in the linear region of the curve (FIG. 2A). For the loss modulus, at lower strain rates, the incorporation of BTO did not affect the viscous behavior (10³ Pa). At higher strain rates, PiezoGEL exhibited a higher loss modulus compared to GelMA (FIG. 2B). A storage modulus of 10⁴ Pa offers a hydrogel with proper mechanical support and stability to periodontal tissues, while a loss modulus of 10³ Pa can absorb mechanical forces aiding tissue healing and reducing stress concentration (Lin et al., 2020, Int. J. Nanomedicine, 15:5377).

To further explore the injectability, the force required to push the plunger of a syringe and extrude the material was measured. Using a syringe connected to a 20-G tip, it was observed that GelMA and PiezoGEL hydrogels had an average injection force of 8.5 N (0.9 kgf) and 12 N (1.2 kgf), respectively (FIG. 2C). The increase in injection force with the addition of BTO was expected because the ceramic fillers increase the viscosity of the solution and add restriction during needle extrusion (Dong et al., 2019, ACS Appl. Bio Mater., 2:5985-5994). Nonetheless, the injection force for PiezoGEL is acceptable for the clinical use (<3 kgf) (Alonso et al., 2021, Polymers (Basel), 13:650).

Real-time photorheological tests were performed to determine the curing times of the hydrogels. Appropriate polymerization reduces cytotoxicity due to the leaching of unreacted monomers or residual photoinitiators and guarantees mechanical stability (Kim et al., 2022, Polymers (Basel), 14:979). The change in the storage modulus with time of PiezoGEL is presented in FIG. 2D. Results showed a typical polymerization curve with a step increase in the first 50 s and reaching a plateau after 120 s of light exposure (Gorsche et al., 2017, Anal. Chem., 89:4958-4968).

The voltage produced by the hydrogels after stimulation with cyclic mechanical loading, which is necessary to produce electrical charges and enable the bioactive effects, was also evaluated. The PiezoGEL produced ~10 mV/mm² under a stimulation of 0.9 MPa (FIG. 2E), which is a stress magnitude found in a clinical setting. As expected, no voltage was produced in the control GelMA (FIG. 2E).

Furthermore, the biodegradation of the hydrogels under physiological conditions (PBS) was also evaluated. Hydrogels used for PD treatment must degrade to “open up” space for the regeneration of new tissue and the reduction of the pocket size (Dong et al., 2019, ACS Appl. Bio Mater., 2:5985-5994). The degradation profile showed a maximum degradation of ~40% for both formulations after 14 days of evaluation (FIG. 2F). No significant differences were observed between the groups on days 7 and 14.

Since the quantity of BTO with respect to GelMA is small, the biodegradation profiles are expected to be similar. The degradation profile of GelMA can be influenced by different media types, including aqueous solutions, enzymatic solutions, and simulated body fluids (Heltmann-Meyer et al., 2021, Biomed. Mater. (Bristol), 16:065004). Enzymatic solutions, such as collagenases, provide accelerated degradation rates, whereas simulated body fluids can provide a more realistic assessment of the hydrogel's stability and degradation rate in vivo (Lin et al., 2006, Infect. Immun., 74:6011-6015; Lamont et al., 2000, Oral Microbiol Immunol., 15:341-349).

### Antibacterial Evaluation in Vitro

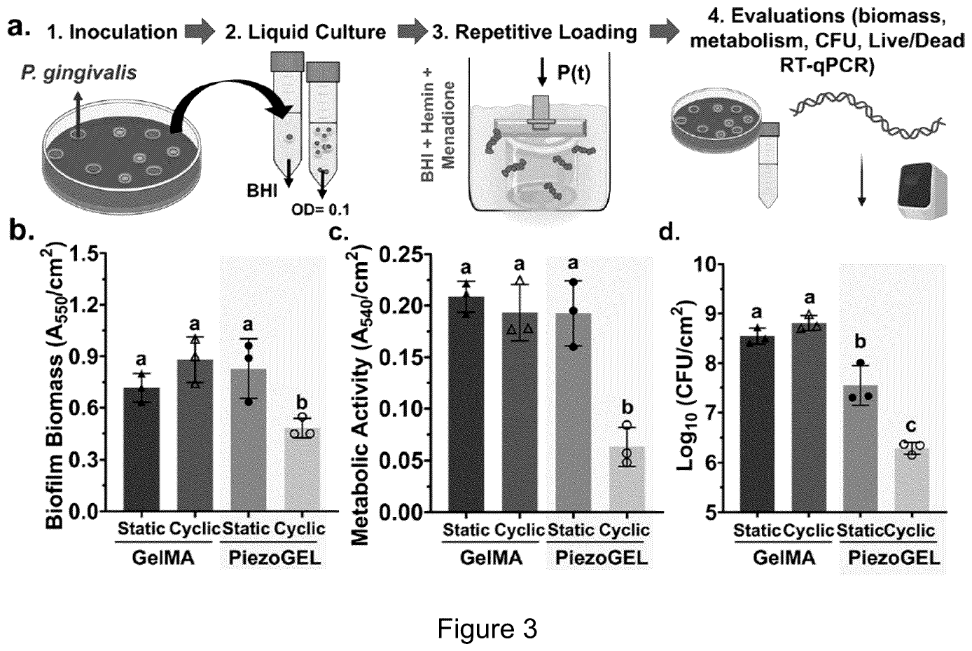

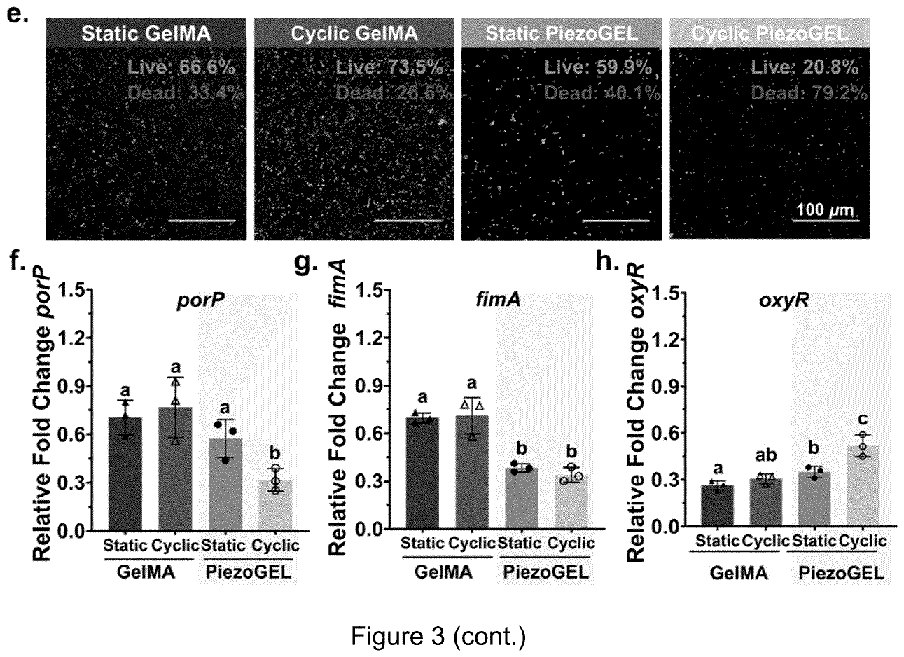

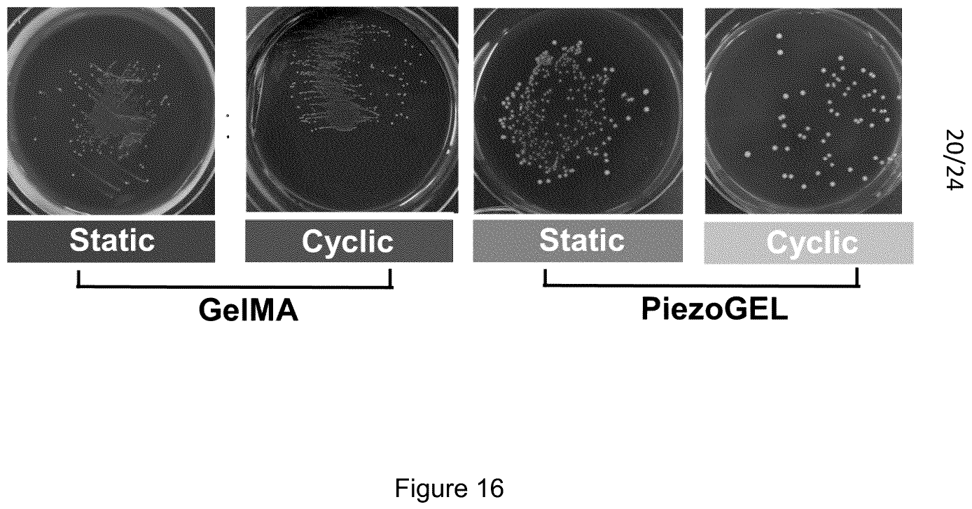

The antibacterial activity of PiezoGEL was assessed against P. gingivalis, a crucial pathogen involved in the development of PD. For these evaluations, hydrogels were subjected to static (no load) and cyclic (repetitive) loading to enable electrical charge generation during the incubation period (FIG. 3A). The hydrogel-bacteria interactions were evaluated by measuring biofilm biomass and metabolic activity and by counting the number of viable cells (CFU). For biofilm biomass, no significant differences were observed in the GelMA hydrogels regardless of the loading condition and the static PiezoGEL (FIG. 3B). However, PiezoGEL stimulated with cyclic loading showed the lowest biofilm biomass. Similar results were obtained for the metabolic responses of the biofilms (FIG. 3C). The lowest reduction was observed in the cyclic PiezoGEL group. These findings indicated that when PiezoGEL activates the electrical charge production via cyclic loading, antibacterial effects are enabled. Regarding CFU, the results showed that most bacteria were found in the GelMA groups (FIG. 3D and FIG. 16), indicating that GelMA did not have any antibacterial effect.

A significant reduction in viable cells was observed for static PiezoGEL (~1 log reduction). However, PiezoGEL stimulated by cyclic loading (charge regeneration) resulted in a 2-3 log reduction (FIG. 3D). Regardless of the mechanical loading conditions, PiezoGEL exhibited a reduction of the bacterial load. These findings were validated by imaging live and dead cells attached to hydrogels (FIG. 3E). The highest number of live cells (green) was observed in the GelMA groups (66-73%). Conversely, cyclic PiezoGEL treatment resulted in the highest number of dead cells (~79%) and the lowest number of live cells. The results confirmed and extend previous findings showing the antimicrobial effect of piezoelectric charges on oral pathogens (Montoya et al., 2021, ACS Appl. Mater. Interfaces, 13:43868-43879; Montoya et al., 2021, ACS Biomater Sci. Eng., 7:4838-4846; Carvalho et al., 2019, ACS Appl. Mater. Interfaces, 11:27297-27305).

To reveal the antibacterial mechanism and understand changes in biofilm formation, the expression of genes related to adhesion, biofilm formation, and virulence were measured. First, the expression of porP, which encodes the biosynthesis of cell-surface polysaccharides, biofilm growth, and gingipain translocation, was measured (Sánchez et al., 2019, BMC Microbiol., 19:58). The results showed that porP was significantly downregulated for the cyclic PiezoGEL compared with the other groups (FIG. 3F). It is known that under normal biofilm growing conditions, porP is upregulated (Sánchez et al., 2019, BMC Microbiol., 19:58) indicating that cyclic PiezoGEL altered the adhesion potential to biomaterial surfaces.

Second, the relative gene expression of fimA, a gene responsible for encoding the major subunit protein of fimbriae, was measured. fimA is related to bacterial colonization and adhesion, and its primary role is to promote bacterial attachment to oral surfaces (Lin et al., 2006, Infect. Immun., 74:6011-6015). Under normal growth conditions, P. gingivalis expresses high levels of fimA, optimizing adherence to available substrates (Lamont et al., 2000, Oral Microbiol Immunol., 15:341-349). The results show a significant downregulation of fimA in the PiezoGEL group regardless of the loading condition (static or cyclic) (FIG. 3G). Previous studies have associated decreased expression of fimA with inhibition of the initial attachment of P. gingivalis to the tooth surface and, therefore, the subsequent formation of biofilm and maturation (Lin et al., 2006, Infect. Immun., 74:6011-6015). The downregulation of fimA in PiezoGEL indicates a reduced amount of biofilm, which is consistent with microbiological findings, such as the reduced cell count (CFU) (FIG. 3D).

Third, changes in oxyR were evaluated since this gene has been associated with an oxidative stress regulator sensitive to hydrogen peroxide (Storz et al., 1992, J. Nutr., 122:627-630; Wu et al., 2008, FEMS Microbiol Lett., 282:188-195). OxyR is crucial for P. gingivalis' aerotolerance and plays a significant role in regulating other vital virulence factors such as biofilm formation (Meuric et al., 2008, Oral Microbiol Immunol., 23:308-314). Previous studies have proposed ROS as the antibacterial mechanism triggered by (piezo)electric charges (Wang et al., 2023, Adv. Mater., 35:e2212315). During the normal respiration of obligatory anaerobes, ROS are produced in the respiratory electron transport chain and cytoplasm (Zou et al., 2022, J. Dent Sci., 17:459-467). Without an ideal redox balance, overproduced ROS can damage essential biological macromolecules, including lipids, proteins, RNA, and DNA (Yamamoto et al., 2011, Appl. Environ. Microbiol., 77:6733-6736). The results showed a significant increase in the expression of oxyR in the cyclic PiezoGEL group compared with the other hydrogel groups (FIG. 3H). This indicated ROS as the antibacterial mechanism of PiezoGEL.

These findings were consistent with similar studies regarding the antimicrobial mechanism of piezoelectric charges (Montoya et al., 2021, ACS Appl. Mater. Interfaces, 13:43868-43879; Montoya et al., 2021, ACS Biomater Sci. Eng., 7:4838-4846; Juan et al., 2021, Int. J. Mol. Sci., 22:4642). The lowest expression of oxyR was observed in GelMA hydrogels, since no electrical charges are produced. The highest expression of oxyR in the cyclic PiezoGEL agreed with the maximum reduction in metabolic activity and the number of viable cells (FIG. 3C and FIG. 3D).

Overall, the results of this study indicated that the electrical charges produced by PiezoGEL enable antibacterial effects against P. gingivalis. A significant reduction in biofilm biomass (~41%), metabolic activity (~75%), and the number of viable bacteria in the biofilm (~2-3 log) was observed compared to hydrogels without charges and BTO fillers. Molecular changes in specific genes indicated that the reduction in biofilm formation and cell viability is related to diminished cell adhesion (i.e., downregulation of porP of fimA) and oxidative stress (i.e., upregulation of oxyR). In this work, a single-species model was used to show the antibacterial effects. Studies have shown that removing virulence factors of P. gingivalis from periodontal pockets can significantly reduce the inflammatory response and promote bone regeneration, leading to improved periodontal health (Ishida et al., 2017, NPJ. Aging Mech. Dis., 3:1-7). The toxin (lipopolysaccharide) induces the production of proinflammatory cytokines/chemokines that trigger bone resorption (Ishida et al., 2017, NPJ. Aging Mech. Dis., 3:1-7). Thus, removing P. gingivalis may stop the inflammatory response leading to tissue destruction (Jepsen et al., 2016, Periodontol. 2000, 71:82-112).

Additional studies focus on validating its effects against various species commonly associated with oral infections. Additional studies also evaluate the efficacy of PiezoGEL in periodontal red complex pathogens such as Treponema denticola, and Tannerella forsythia, to have a better understanding of possible microbial dynamics and microbiome changes derived from the biomaterial.

### Bone-Tissue Regeneration Evaluation in Vitro

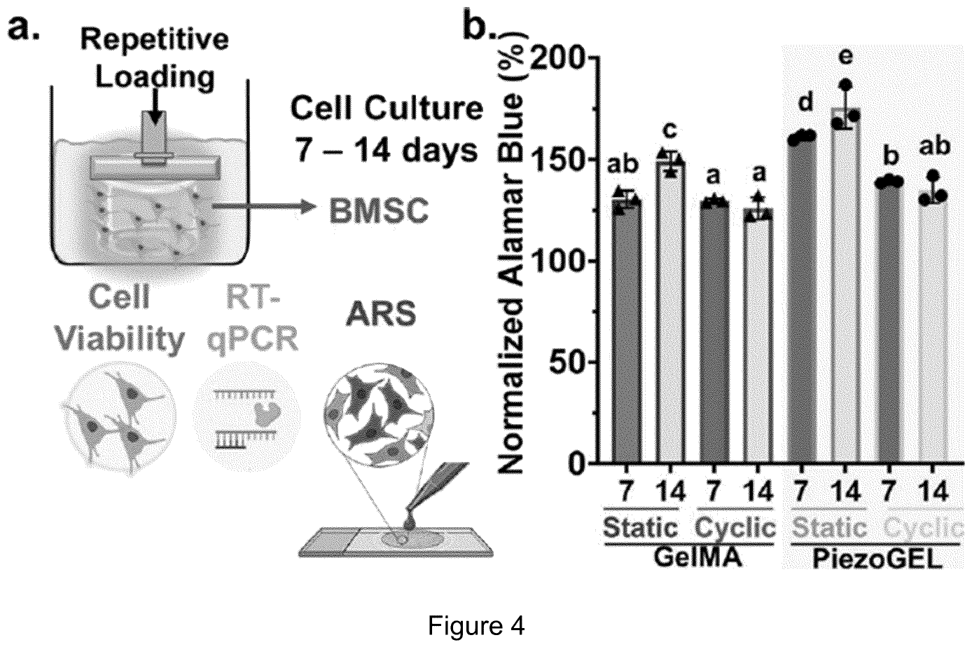

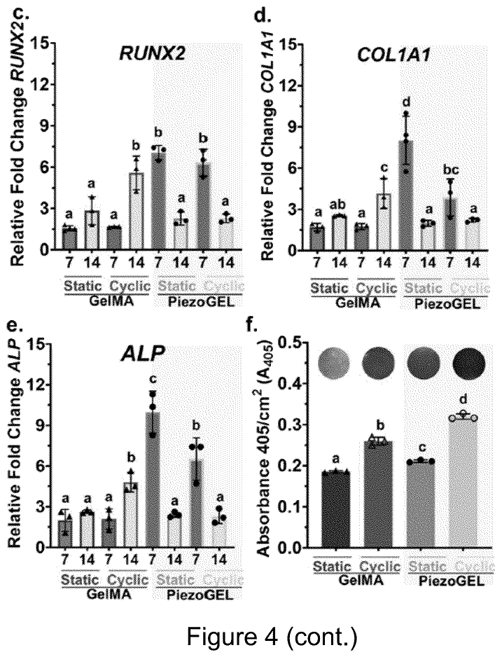

To explore the bone tissue regeneration of PiezoGEL, cell viability, osteogenic differentiation, and mineral formation were evaluanted after BMSCs were cultivated in hydrogels under static and loaded conditions for 7 and 14 days (FIG. 4A). Overall, a positive increase in cell viability of both hydrogel groups was observed, indicating no cytotoxicity of these biomaterials (FIG. 4B).

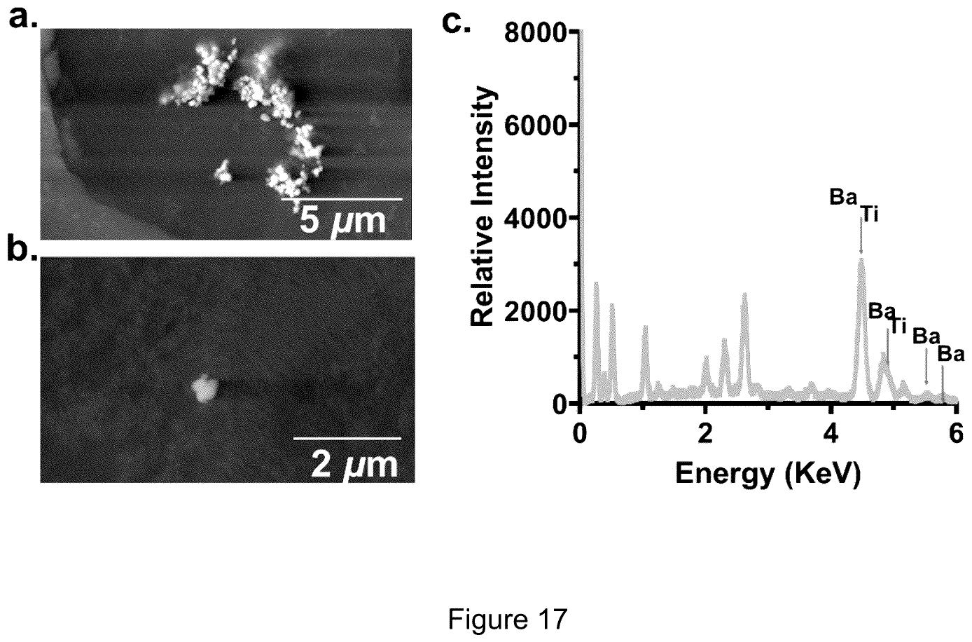

The normalized reduction of alamarBlue >100% for all hydrogels (GelMA and PiezoGEL) indicated superior viability compared to that of the cells cultured in the empty well (reference used in normalization). The results showed that the viability of PiezoGEL was significantly higher than that of GelMA for all evaluated days and loading conditions (FIG. 4B). The results indicated that the addition of BTO positively affects cell proliferation. Interestingly, PiezoGEL under static conditions showed the highest viability. Although not bound by any particular theory, it was hypothesized that the cyclic mechanical stimulation of the PiezoGEL speeds up the biodegradation process due to the continuous movement forcing the additional leaching of BTO fillers, which reduces the electrical charge production (FIG. 17).

Looking into the expression of early markers of osteogenic differentiation, relevant BMSC genes, including RUNX2, COL1A1, and ALP, were examined. The gene RUNX2 is considered a master regulator of osteogenesis. It plays a crucial role in initiating the osteogenic differentiation process (Liu et al., 2013, Tissue Eng. Part B Rev., 19:254-263). This gene was significantly upregulated by PiezoGEL on day 7 with a downregulation on day 14 (FIG. 4C). The early expression of RUNX2 indicated the quick degree of commitment and differentiation of BMSCs toward the osteoblast lineage and suggested the activation of other osteogenic genes and signaling pathways necessary for premature bone formation (Komori et al., 2009, Adv. Exp. Med. Biol., 658:43-49).

Interestingly, the static PiezoGEL group (no piezoelectric charge generation) also showed early expression of RUNX2. It is known that BTO particles alone accelerate osteogenesis (Furuya et al., 2011, Proc. SPIE, 7975:79750U). Similar to cell viability, the leaching of BTO particles in loaded PiezoGEL can influence the early differentiation process. Thus, additional studies focus on deconvoluting the effective effect of piezoelectric charges and BTO fillers on osteogenic differentiation.

Overall, hydrogels of GelMA did not significantly upregulate RUNX2. However, GelMA under cyclic loading for 14 days showed a significant upregulation. Cyclic loading of biomaterials has shown to induce osteogenic differentiation of BMSCs via Wnt/β-catenin signaling pathway (Liu et al., 2021, Front. Bioeng. Biotechnol., doi: 10.3389/fbioe.2021.735949; Lin et al., 2019, ACS Biomater Sci. Eng., 5:6446-6453) which stimulated the expression of ALP and RUNX2 (Krause et al., 2010, Proc. Natl. Acad. Sci. U.S.A., 107:4147-4152).

A similar trend was observed in the expression of COL1A1 and ALP. COL1A1 is the major component of the ECM in bone. Its early expression is upregulated during osteogenic differentiation and is crucial for bone matrix synthesis (Vimalraj et al., 2020, Gene, 754:144855). Under both static and cyclic loading conditions, PiezoGEL demonstrated early differentiation of COL1A1 within 7 days (FIG. 4D), indicating the production of the initial extracellular matrix (ECM) essential for bone formation. The upregulation of COL1A1 in PiezoGEL is a downstream effect of the RUNX2 activation, which triggers genes involved in osteoblast differentiation (Komori et al., 2010, Cell Tissue Res., 339:189-195).

Notably, the upregulation of COL1A1 at day 7 from the cyclic PiezoGEL was significantly lower than that from the static group. It appears that the electrical charge generation from the cyclic loading accelerated the upregulation of early osteogenic gene expression. Genes' analysis at early time points (days 1 and 3) could explain this phenomenon. ALP is an enzyme that is highly expressed during osteogenic differentiation, and its expression increases as BMSCs differentiate into preosteoblasts and osteoblasts. ALP is involved in mineralization (Vimalraj et al., 2020, Gene, 754:144855). The gene expression trend between ALP and RUNX2 is similar. For PiezoGEL, there is higher upregulation at day 7, whereas there was no regulation for GelMA (FIG. 4E). ALP expression on day 7 indicated early osteoblast maturation and bone formation. However, as the bone formation process progresses, the activity of ALP may decrease as osteoblasts mature and mineralize the bone matrix.

Taken together, PiezoGEL induced the early expression of critical markers of bone formation, indicating a quick osteogenic differentiation. Both piezoelectric charges and BTO fillers are likely contributing to these responses. BTO generates electrical charges in response to mechanical stress (Levin et al., 2014, Mol. Biol. Cell, 25:3835-3850). This electrical charge stimulates BMSC to differentiate, proliferate, and form new bone tissue. Electrical charge can also stimulate angiogenesis necessary for bone regeneration (Leppik et al., 2020, European Journal of Trauma and Emergency Surgery, 46:231-244). Electric charges are known to affect cell behavior, including growth, migration, and differentiation (Levin et al., 2014, Mol. Biol. Cell, 25:3835-3850). These charges can stimulate the differentiation of stem cells into specific types of cells, such as bone-type cells, by influencing the flow of ions across the cell membrane, which activates signaling pathways involved in cell differentiation (Heng et al., 2022, Smart Mater. Med., 3:4-11; Harris et al., 2021, Development, 148:dev180794).

This is the first attempt to use piezoelectrical charges for periodontal bone tissue regeneration. BMSCs have been studied extensively for periodontal bone tissue regeneration due to the multilineage differentiation potential, are easily accessible, and exhibit a higher growth capacity than PDL cells (Li et al., 2020, Stem Cell Res. Ther., 11:1-15).

To study late-stage markers of bone formation, the ECM mineralization at day 14 was evaluated (Hayashi et al., 2008, Calcif Tissue Int., 82:238-247). A significant amount of mineral was noted for all of the hydrogels (FIG. 4F). Specifically, cyclic PiezoGEL hydrogels exhibited the highest level of ECM mineralization, followed by the cyclic GelMA group. The lowest ECM mineralization was measured for the static GelMA group. These results are consistent with those showing the expression of ALP (FIG. 4E), indicating that hydrogels under cyclic strain induced an early expression.

In addition, ECM mineralization from PiezoGEL explains why the expression of RUNX2, COL1A1, and ALP on day 14 was not upregulated since it is possible that the peaks in expression occurred prior. In addition, ECM of interest, GelMA exhibited ECM mineralization which is consistent with previous reports (Shi et al., 2020, Int. J. Nanomedicine, 15:9337-9353).

### Periodontal Bone Regeneration and Inflammatory Evaluations In Vivo

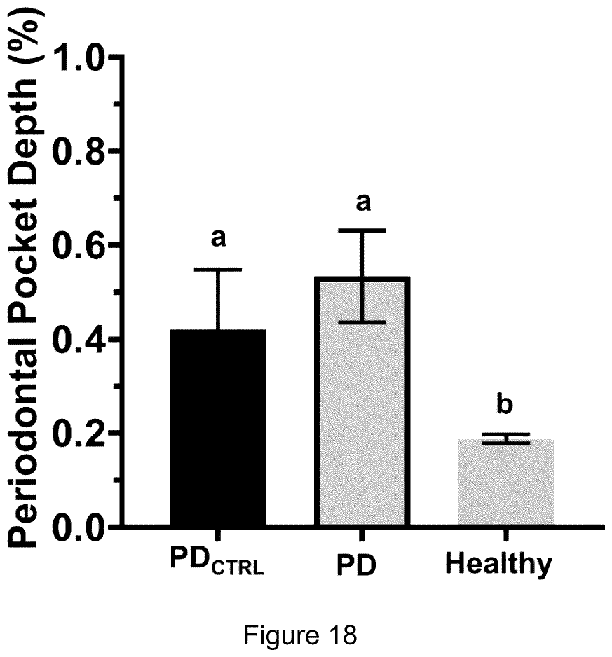

PD and bone loss were successfully induced in mice after placing a ligature around the molars for a week. An initial clinical evaluation for bleeding and PD confirmed the induction of the disease for all the groups. Healthy and diseased pocket depths were 0.2 and 0.55 mm, respectively. In addition, micro-CT evaluation showed a distance between the CEJ and ABC for healthy and diseased animals of 0.18 mm and 0.60 mm, respectively (FIG. 18). The results were consistent with previous studies showing that the pocket depth after 1 week of ligature-induced PD is ~0.60 mm (Li et al., 2020, Front. Bioeng. Biotechnol., 8:444). No adverse reactions, such as local/systemic toxic symptoms or weight loss, were observed during treatment.

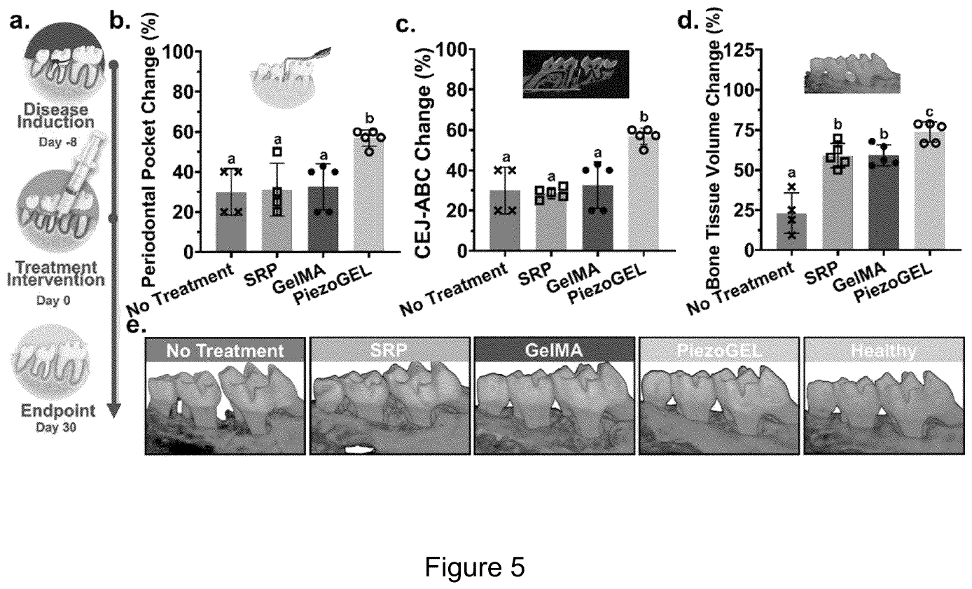

After 1 month of treatment, changes in pocket depth, bone tissue, and inflammation were evaluated. PiezoGEL showed the highest change in pocket depth among all other evaluated groups (FIG. 5B). The final pocket size obtained for PiezoGEL after treatment was 0.2 mm, which is comparable to that under healthy conditions (FIG. 18). The pocket depth changes for GelMA, SRP, and no treatment were similar. These results were consistent with the fact that no bioactive therapies were provided in these groups. The robust immune response and natural regenerative abilities of mice may explain why the no treatment group rendered similar pocket depth change (Masopust et al., 2017, Journal of Immunology, 199:383-388).

Looking into the CEJ to ABC distance, again, PiezoGEL showed the highest change among other groups (FIG. 5C). In other words, more bone tissue was regenerated after treatment. These results indicated that increased bone tissue regenerated by PiezoGEL helped reduce the size of the pocket and, thus, rendered a bigger change after treatment. GelMA, SRP, and no treatment groups showed a similar amount of regenerated bone and 50% less than PiezoGEL.

Looking into the regenerated bone tissue volume, PiezoGEL showed the highest change among the other groups (FIG. 5D). The no treatment group showed the lowest bone volume (20%), indicating the lowest amount of bone regeneration. The SRP and GelMA groups showed a similar amount of regenerated bone volume (55%), which was higher than no treatment but lower than PiezoGEL.

Taken all together, these results indicated that PiezoGEL can augment bone tissue around the periodontium and reduce the pocket size without the need of surgery, bone grafts, or guided membranes. The 3D reconstruction of the maxilla for all groups confirmed previous observations (FIG. 5E). Improved bone tissue quality and regeneration were observed for PiezoGEL around M1-M2 (location of the defect). These qualities are comparable to those in healthy conditions. No treatment and GelMA showed the poorest bone tissue quality. Different studies have used GelMA as a carrier of bioactive biomaterials for periodontal bone tissue regeneration, including ZIF8 (Liu et al., 2022, Acta Biomater., 146:37-48), hydroxyapatite (Chen et al., 2016, Int. J. Nanomedicine, 11:4707-4718), bioactive glass (Chen et al., 2016, Int. J. Nanomedicine, 11:4707-4718), and BMP2 (bone morphogenetic protein 2) (Chen et al., 2016, Int. J. Nanomedicine, 11:4707-4718). These biomaterials establish a suitable microenvironment around the periodontal tissues and enable the delivery of bioactive therapy to promote bone regeneration.

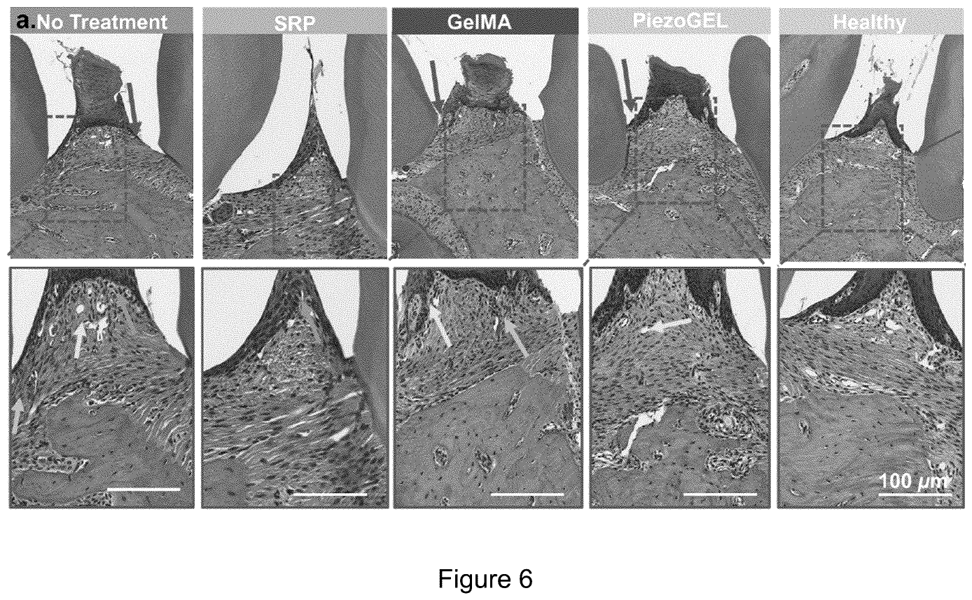

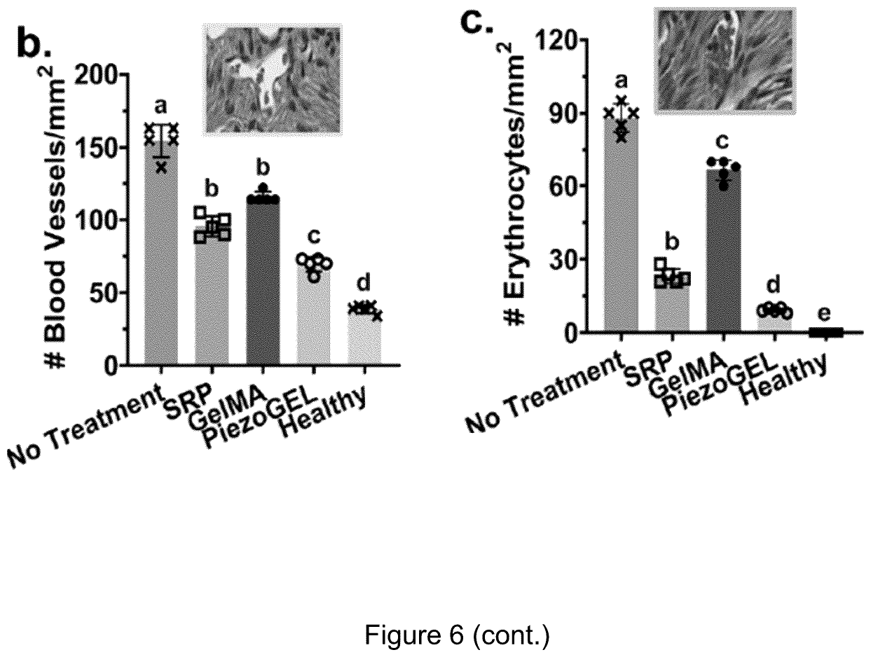

Histological sections stained with H&E were used to evaluate the inflammatory response, cell infiltration, and morphological changes in periodontal tissues caused by PiezoGEL. Histopathological changes were used to evaluate the effectiveness of PiezoGEL in reducing bacterial load and infection in the tissue. The results of H&E staining supported the trends observed by micro-CT (FIG. 6). In general, PD showed changes in epithelial detachment, epithelial hyperplasia, disorganization of collagen fibers, and erythrocyte density (Ionel et al., 2017, Histol. Histopathol., 32:379-384).

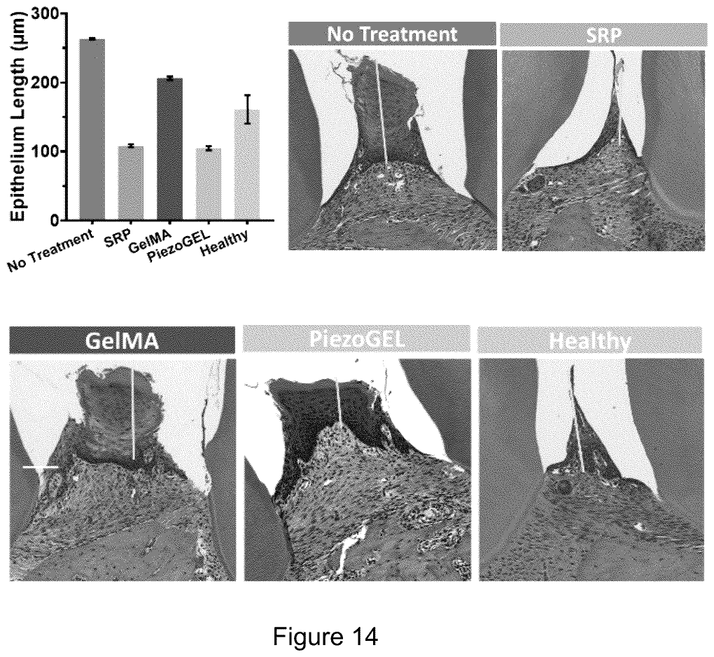

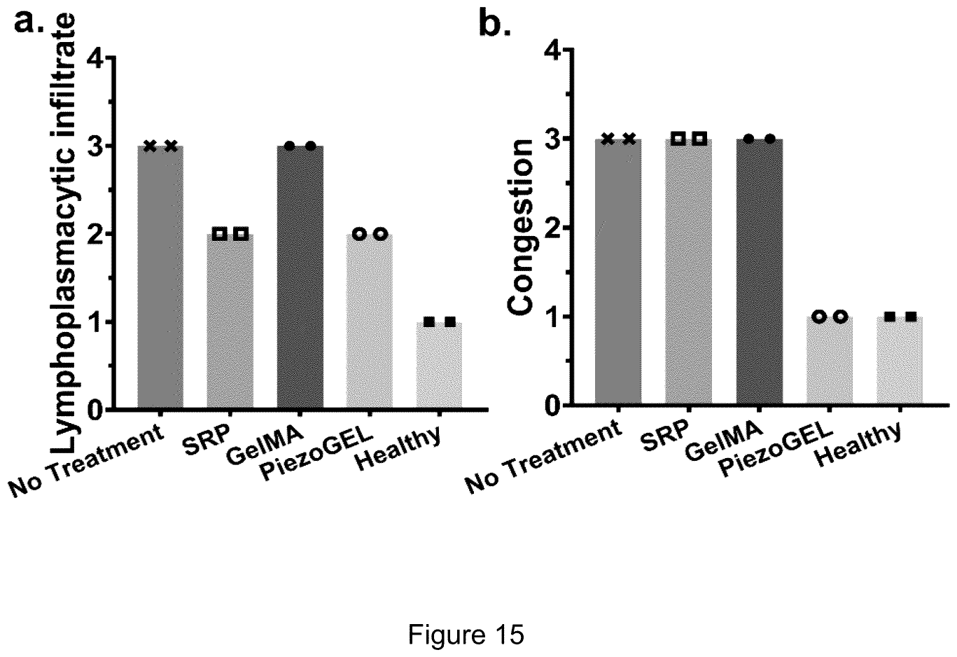

Significant tissue destruction associated with chronic inflammation was observed in the GelMA and no treatment groups, while PiezoGEL and healthy groups showed, in general, a junctional epithelial union (red arrow, FIG. 6A). Additionally, PiezoGEL and healthy groups showed decreased epithelial length (FIG. 14), inflammatory assessment, and lymphoplasmic infiltrate (FIG. 15A). The inflammatory response was quantified by counting the number of blood vessels (yellow arrows) and erythrocytes (green arrows) in the infected area between M1 and M2 (insets in FIG. 6A).

A higher number of blood vessels was counted in the no treatment group (~160 blood vessels/mm²), followed by GelMA (~120 blood vessels/mm²) (FIG. 6B). Lower blood vessel count was found in the healthy (~40 blood vessels/mm²) and PiezoGEL groups (73 blood vessels/mm²). A higher number of blood vessels is associated with an ongoing inflammatory process in which new blood vessels are formed to supply oxygen and nutrients to the affected area (Pahwa et al., 2023, Chronic Inflammation; StatPearls: Treasure Island, FL).

In PD, the increased number of blood vessels is often accompanied by the influence of immune cells, such as neutrophils (blue arrows in insets of FIG. 6A), which exacerbate the inflammatory response and lead to tissue destruction (Cortés-Vieyra et al., 2016, J. Immunol Res., 1396106). Dogs with PD showed an average number of blood vessels per mm² of 128, whereas it was 63 for treated (cured) tissues (Imber et al., 2021, Int. J. Mol. Sci., 22:10915). These counts are consistent with the current findings.

Similar to the number of blood vessels, a low number of erythrocytes is associated with healthy periodontal tissues. In this study, lower erythrocyte counts were observed in the healthy and PiezoGEL groups (FIG. 6C). Higher counts of erythrocytes were observed in the untreated group. A higher red blood cell count indicates increased vascular permeability and blood flow in the affected area, which can result from an inflammatory response and tissue damage (Pahwa et al., 2023, Chronic Inflammation; StatPearls: Treasure Island, FL).

Overall, the results indicated that PiezoGEL was able to decrease inflammation compared to healthy tissues. PiezoGEL successfully achieved clinical attachment level by improving pocket depth and recovering lost bone volume. Evaluation of these parameters is known to determine the success of periodontal regenerative therapy (Chen et al., 2016, Int. J. Nanomedicine, 11:4707-4718).

The current standard of care for the nonsurgical treatment of PD includes SRP in combination with adjuvants (i.e., antibacterial agents) (Khattri et al., 2020, Cochrane Database Syst. Rev., 11:CD012568; Liang et al., 2020, Bioact Mater., 5:297-308). However, clinically SRP fails to reduce the pocket size owing to insufficient antibacterial effects and the lack of natural regeneration of periodontal tissues (Van der Weijden et al., 2019, Int. J. Dent Hyg., 17:309-317). Additionally, surgical approaches due to their high cost and invasiveness are limited to severe periodontitis. Recently, hydrogels have been used to treat PD because of their ability to encapsulate and release therapeutic agents in a controlled manner (Ye et al., 2022, Gels, 8:302). However, one of the major challenges for these hydrogels in treating PD is the need to effectively deliver combined antibacterial agents and regenerative therapies to the site of infection.

The herein described hydrogel exhibited unique advantages as a single-use material in reaching periodontal defects and adapting to its shape, while providing dual antibacterial and regenerative effects. Bioactive therapies were provided using nonleaching technologies, which can overcome the limitations of current technologies.

Moreover, there are several advantages of PiezoGEL over the current standards of care. First, the technology is fundamentally different from existing adjuvants and treatments for PD since it offers a drug-free antibacterial treatment without raising concerns regarding bacterial antibiotic resistance. Second, PiezoGEL has a simple formulation with only three ingredients: GelMA (a common hydrogel approved for medical use) (Piao et al., 2021, Engineered Regeneration, 2:47-56), piezoelectric fillers, and a photoinitiator. The benefits of hydrogels (injectable, light curable, conforms to pocket spaces, and biocompatibility) were harnessed for delivery into the periodontal pocket. Third, PiezoGEL offered bone tissue regeneration without surgery, which is a novel concept in the nonsurgical treatment of PD.

Some of the potential limitations of PiezoGEL are the possible short residency if the adhesion is poor and the control of the electrical charges to elicit therapies. The surgical treatment of PD from a biomaterials perspective relies on bone grafts, guided tissue regeneration (GTR), and tissue engineering with controlled delivery of drugs or growth factors (Liang et al., 2020, Bioact Mater., 5:297-308). Surgical approaches are invasive, technically challenging, with a high cost, and usually recommended when nonsurgical approaches are exhausted.

Findings from this work begin to pave the way for the clinical use of PiezoGEL in treating periodontitis. To translate the technology, additional studies focus on additional biocompatibility tests based on ISO-10993, including oral toxicity, systemic toxicity, oral mucosal irrigation, and USP pyrogen. In addition, studying the adhesion of the hydrogel inside the pocket in vivo to determine the number of applications required for treatment is also being conducted. Also, additional degradation tests in vivo explore the range of applications necessary for various patient populations and the severity of PD. Overall, PiezoGEL represents a significant advancement in PD treatment and improvement of patient outcomes in periodontal regenerative medicine.

In conclusion, this study successfully developed an injectable piezoelectric hydrogel (PiezoGEL) for the nonsurgical treatment of periodontal disease by combining GelMA with barium titanate fillers. PiezoGEL exhibited remarkable antibacterial and bone-regeneration effects both vitro and in vivo. For antibacterial evaluations, PiezoGEL demonstrated a significant reduction in biofilm formation and viable cells against P. gingivalis compared to GelMA hydrogels in vitro.

Moreover, PiezoGEL upregulated the expression of osteogenesis-related genes (RUNX2, COL1A1, and ALP) and promoted early ECM mineralization. In vivo, PiezoGEL relieved inflammation, promoted alveolar bone tissue regeneration, and reduced the periodontal pocket depth after 1 month of treatment. Results from both in vitro and in vivo investigations strongly indicated the efficacy of PiezoGEL as a promising bioactive material for the treatment of periodontal disease by combining antibacterial properties, osteogenic potential, and the ability to reduce inflammation.

In summary, periodontal disease is a multifactorial, bacterially induced inflammatory condition characterized by the progressive destruction of periodontal tissues. The successful nonsurgical treatment of periodontitis requires multifunctional technologies offering antibacterial therapies and promotion of bone regeneration simultaneously. For the first time, in this study, an injectable PiezoGEL was developed after combining GelMA with biocompatible piezoelectric fillers of BTO that produce electrical charges when stimulated by biomechanical vibrations (e.g., mastication, movements). The benefits of hydrogels (injectable, light curable, conforms to pocket spaces, biocompatible) were harnessed with the bioactive effects of piezoelectric charges. A thorough biomaterial characterization confirmed piezoelectric fillers successful integration with the hydrogel, photopolymerizability, injectability for clinical use, and electrical charge generation to enable bioactive effects (antibacterial and bone tissue regeneration).

PiezoGEL showed significant reductions in pathogenic biofilm biomass (~41%), metabolic activity (~75%), and the number of viable cells (~2-3 log) compared to hydrogels without BTO fillers in vitro. Molecular analysis related the antibacterial effects to be associated with reduced cell adhesion (downregulation of porP andfimA) and increased oxidative stress (upregulation of oxyR) genes. Moreover, PiezoGEL significantly enhanced bone marrow stem cell (BMSC) viability and osteogenic differentiation by upregulating RUNX2, COL1A1, and ALP. In vivo, PiezoGEL effectively reduced periodontal inflammation and increased bone tissue regeneration compared to control groups in a mice model. Findings from this study suggest PiezoGEL to be a promising and novel therapeutic candidate for the treatment of periodontal disease nonsurgically.

The materials and methods employed in these experiments are now described.

### Fabrication of Piezoelectric Hydrogels

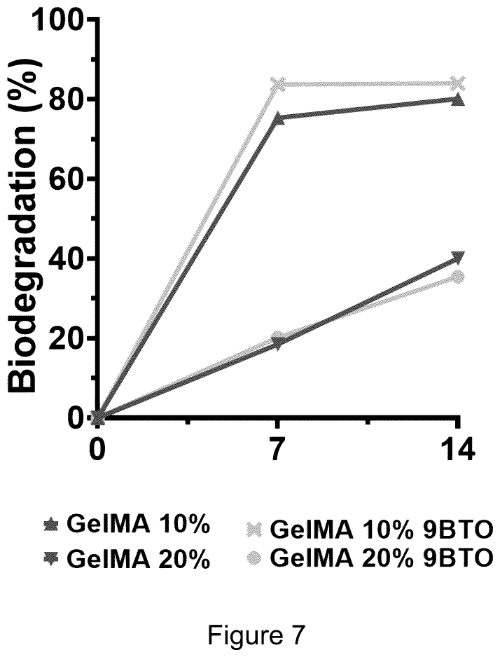

PiezoGEL was fabricated by mixing 200 mg/mL GelMA (Advanced Biomaterials, 5208) with 9 mg/mL silanized particles of BTO (US Nanomaterials, U.S. Pat. No. 3,830,200 nm) and sterile phosphate-buffered saline (PBS) (FIG. 7). This filler size offered an appropriate balance between the injectability and electrical charge generation. Increasing this particle size would increase the hydrogel viscosity and injection force and lower the degradation rate. The photoinitiator lithium phenyl-2,4,6-trimethylbenzoyl phosphonate (LAP) (Advanced Biomaterials, 5269) was added at a proportion of 0.35% wt of the solubilized GelMA. Lyophilized GelMA and PBS (70° C.) were mixed using a vortex mixer (Daigger Vortex-Genie) until GelMA was fully solubilized. BTO particles were added during the continuous shaking of the mixture. Subsequently, LAP was dissolved in 100 μL of warm PBS (70° C.) and mixed with the dissolved GelMA/BTO mixture. Control hydrogels were prepared without BTO filler (GelMA). The mixture was poured into a 6 mL polypropylene syringe, which was used as a mold for the uncured solution.

After placing in the syringe, a 405 nm visible light was positioned at 1 cm of the solution and irradiated for 2 min, resulting in a total radiant exposure of 24.5 J/cm². After curing, the samples were carefully demolded, cut into cylinders (12 mm diameter and 6 mm thickness), and stored in DI water for 24 h at 4° C. for the release of unreacted monomers. The hydrogels were prepared inside a biological safety cabinet and sterilized using a UV lamp (254 nm) for 15 min before each test. Samples were placed 30 cm from the UV source.

### Physical, Mechanical, and Electromechanical Characterization of Hydrogels

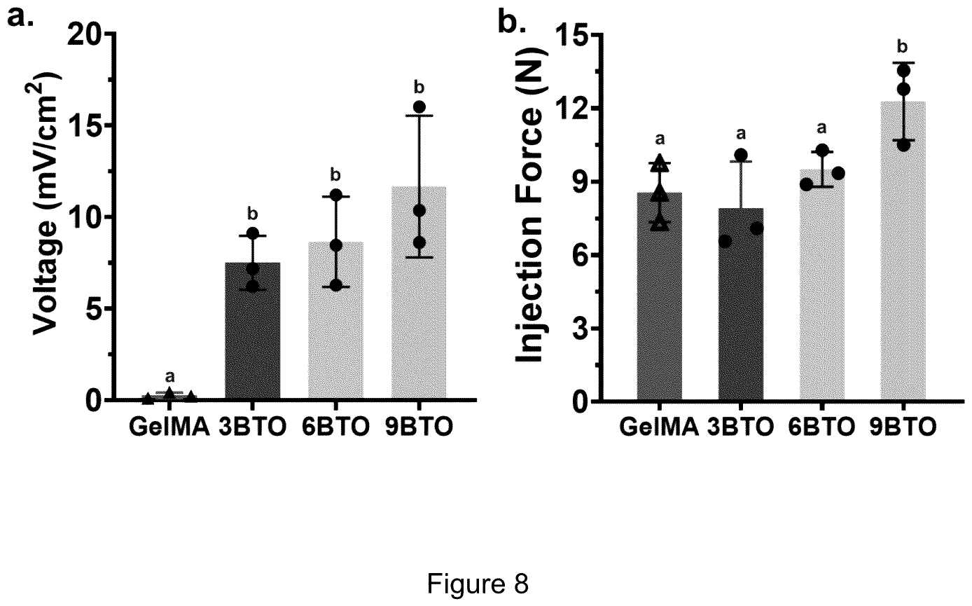

The physical, mechanical, and electromechanical properties of PiezoGEL, including the surface pore size, surface roughness, rheology, injectability, and biodegradation were evaluated (FIG. 8 through FIG. 10).

Rheological properties of the hydrogels were assessed using a Rheological StressTech HR rheometer (ATS RheoSystems) with parallel plate geometry (25 mm diameter and 1 mm gap) and samples maintained at 37° C. (Alonso et al., 20221, Polymers (Basel), 13:650).

The storage and loss moduli of the cured hydrogels were measured by conducting a strain sweep from 0.01% to 500% at 5 Hz with a conditioning time of 3 s, sampling time of 3 s, and 10 points/decade. This is the standard protocol for measuring the rheology of hydrogels for clinical applications (Valero et al., 2018, PLoS One, 13:e0195820).

To determine the curing time, rheological tests were performed with 200 μL of uncured GelMA while the liquid mixture was cured using a lamp. Changes in the storage modulus during polymerization were measured as previously described (Valero et al., 2018, PLoS One, 13:e0195820; González et al., 2020, Nanomaterials (Basel), 10:1788).

Injectability was measured by placing 500 μL of a liquid mixture of GelMA or PiezoGEL in a disposable syringe (1 mL) connected to a 20-G½ tip without air bubbles. The syringe was placed in a rig coupled to a universal testing machine (Electroforce 5500) (Moreira et al., 2018, Mater. Chem. Phys., 218:304-316). The machine cross-head was used to push the syringe plunger and simulate an extrusion flow rate of 2 mL/h (cross-head of 0.45 mm/s). The force applied to the plunger during injection was recorded.

Hydrogels' voltage/electrical charge generation was measured by placing electrodes (thin copper sheets) on the top and bottom surfaces of the cured hydrogels. The electrodes were connected to a piezoelectric amplifier (TE Connectivity Piezo Film Lab Amplifier) to measure the open-circuit voltage. The hydrogels were then cyclically loaded using a universal testing machine, following a sinusoidal waveform (3 N, 2 Hz). The voltage was recorded during the stimulation.

The degradation of the hydrogels was performed after preparing the disks (12 mm diameter and 6 mm thickness) and placing them in DI water for 24 h. Subsequently, the disks were dried under a vacuum until they reached a constant weight and then submerged in PBS (pH 7.4) for 14 days at 37° C. The medium was refreshed every day. Dry weight loss was monitored as a function of time (0, 7, and 14 days). Weight loss (A W %) was calculated as the change in weight (initial−final)/(initial) (Yuan et al., 2020, ACS Appl. Mater. Interfaces, 12:3382-3392). For all evaluations, three samples from independent batches were evaluated.

### Antibacterial Evaluation in Vitro

P. gingivalis Biofilm Model

To evaluate the antibacterial effects of the hydrogels, a single-species biofilm model was used following the established protocols (Montoya et al., 2021, ACS Appl. Mater. Interfaces, 13:43868-43879). P. gingivalis was used as the test bacterium because it is the most important pathogenic bacterium in chronic PD (Sánchez et al., 2019, BMC Microbiol., 19:58; How et al., 2016, Front. Microbiol., doi: 10.3389/fmicb.2016.00053). This pathogen produces lipopolysaccharides that induce proinflammatory cytokines/chemokines that cause bone loss (Lian et al., 2018, Mol. Immunol., 103:209-219). The strain ATCC 33277 was plated on prereduced sheep blood agar supplemented with hemin (5.0 g/mL) and menadione (0.5 g/mL) and incubated under anaerobic conditions (5% H₂, 5% CO₂, 90% N₂) for 4 days at 37° C. (Mendez et al., 2019, Front Cell Infect Microbiol., 9:246).

To create the bacterial liquid culture, a single colony was harvested and incubated anaerobically overnight in reduced/stirred supplemented brain heart infusion (BHI). The solution was diluted in BHI to obtain OD 600 nm=0.1 (~1×10⁸ cells/mL). Sterile-cured hydrogels were submerged in 3 mL of bacterial culture and subjected to cyclic loading (Cell Scale MechanoCulture TX) throughout the incubation period (24 h). During the experiment, the load magnitude was adjusted from −0.5 to −3 N, resembling the clinical stress in the periodontium (~0.90 MPa, frequency of 2 Hz) (Nikolaus et al., 2017, J. Mech Behav Biomed Mater., 67:61-73; Yoshida et al., 2001, Med. Eng. Phys., 23:567-572). Static hydrogels were also used. After incubation, the top and bottom of the hydrogel were cleaned with a blade. Samples (N=3 per group per evaluation) were gently rinsed with PBS to remove unattached cells and used to evaluate biofilm formation.

### Microbiological Biofilm—Biomaterial Evaluations

To assess the biomass, metabolic activity, and a number of viable cells in the biofilms, crystal violet (CV), [3-(4,5-dimethylthiazol-2-yl)-2,5-diphenyltetrazolium bromide](MTT) assay and counting of colony forming units (CFU) were performed according to previously established protocols (Montoya et al., 2021, ACS Appl. Mater. Interfaces, 13:43868-43879; Camilleri et al., 2020, Dental Materials, 36:e59-e73). To visualize and quantify live and dead bacteria on the hydrogels, the cells were stained by using the LIVE/DEAD BacLight viability kit (Thermo Fisher L7007). Briefly, the hydrogels were placed in 1 mL of PBS and vortexed for 1 min to detach the biofilm. Cells were collected by centrifugation (12 g for 5 min at room temperature) and stained with a fluorescent stain composed of 3 μL of SYTO9 and 3 μL of propidium iodide (PI) dissolved in 1 mL of ultrapure water (final concentration 6 μM for SYTO 9 and 30 μM for propidium iodide).

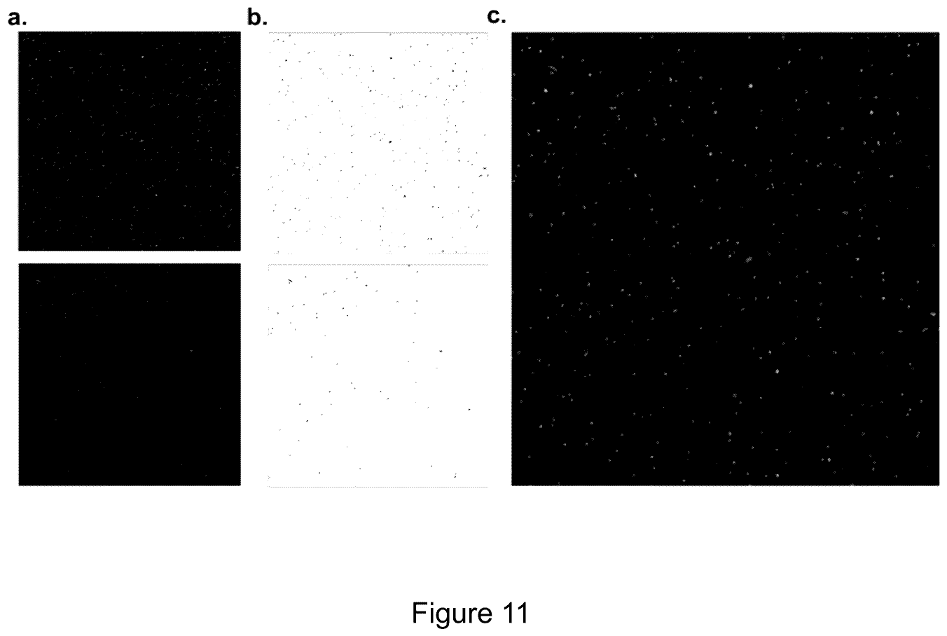

Aliquots (50 μL) of the stain were applied to the bacterial pellets, and the pellets were incubated in the dark for 20 min. The bacterial pellet was gently rinsed with filter-sterilized water to remove excess dye. The cells were imaged using a confocal laser scanning microscope (CLSM) (Olympus Corporation, FV1200). Five experimental images were obtained for each sample. All intensities were corrected for background fluorescence and captured using identical settings. The numbers of live and dead bacteria were quantified using ImageJ software (FIG. 11).

### Molecular Biofilm—Biomaterial Evaluations: RNA Extraction and Gene Expression Assays

Total RNA was extracted from biofilms after 24 h of incubation using the RNeasy Mini Kit (QIAGEN) according to the manufacturer's instructions. The RNA yield and quality were assessed using a NanoDrop instrument (Thermo Fisher Scientific). cDNA synthesis was performed using the RT2 First Strand Kit (Qiagen, Hilden, Germany) according to the manufacturer's protocol. For cDNA synthesis, 100 ng of RNA was used. Reverse transcription quantitative polymerase chain reaction (RT-qPCR) was performed by using a Bio-Rad CFX96 thermocycler (Bio-Rad, USA) and an iTaq Universal SYBR Green Supermix (Bio-Rad, USA). Gene-specific primers associated with pathogenicity and biofilm formation were used, including fimA, porP, and oxyR (Thermo Fisher Scientific) (Table 1) (Sharaf et al., 2023, Microorganisms, 11:15).

TABLE 1
Primer sequences used for RTq-PCR analysis of P. gingivalis cells.

| Name | Function | Primer Sequence |
|---|---|---|
| 16S rRNA | Housekeeping | F 5′-3′: TGT AGA TGA CTG ATG GTG AAA ACC |
| | | R 5′-3′: ACG TCA TCC CCA CCT TCC TC |
| fimA | Promote bacterial | F 5′-3′: GGAATTCCGACGCTATATGCAAGACAA |
| | attachment, invasion, and | R 5′-3′: TGTAACGGTTCTGCCTCGT |
| | colonization | |
| porP | Membrane envelope | F 5′-3′: GGGTAGTGACCGAAACGAGA |
| | | R 5′-3′: CTCCGGGCTTCTCTGTGTT |
| oxyR | Peroxide-sensing | F 5′-3′: CCACAACTGACCGAGAGCA |
| | transcription activator | R 5′-3′: CCTGTCTGCAACTTGTGCAT |

F: Forward primer.
R: reverse primer.

16s rRNA was used as the housekeeping gene (Romero-Lastra et al., 2019, PLoS One, 14:e0221234). RT-qPCR was performed as follows: initial denaturation at 95° C. for 30 s, followed by 40 thermal cycles at 95° C. for 10 s and 60° C. for 30 s. Gene expression was normalized using the comparative Ct (ΔΔCt) method (Rao et al., 2013, Biostat. Bioinf. Biomath., 3:71). Each experimental group and gene primer were analyzed in triplicates.

### Bone-Tissue Regeneration Evaluation in Vitro

### Cell Culture and Seeding

Bone marrow-derived mesenchymal stem cells (BMSCs) were purchased from ATCC (PCS-500-012) and cultured in stem cell basal medium (PCS-500-030) supplemented with stem cell growth medium (PCS-500-040), penicillin, streptomycin, and amphotericin (PCS-999-002). BMSCs were used because they are the most common cell source for bone tissue regeneration and have demonstrated the ability to regenerate periodontal tissue (Raju et al., 2020, Sci. Rep., 10:1-16).

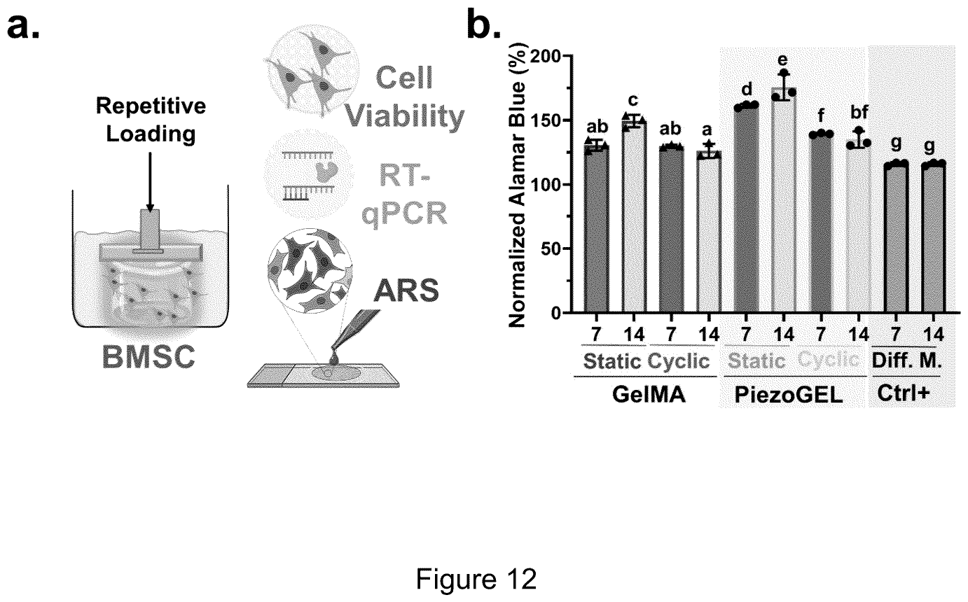

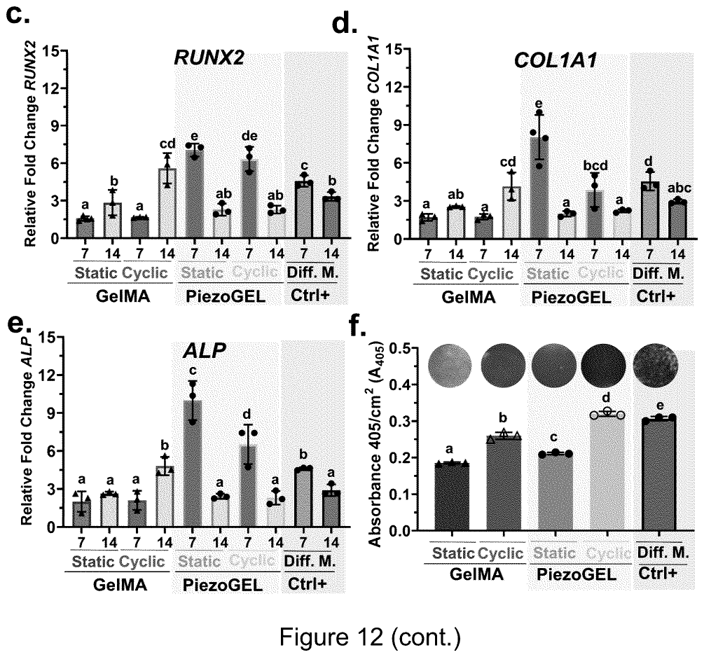

The cells were incubated at 37° C. and 5% CO₂ in a humidified incubator. In this case, static hydrogels were cured directly in 12-well plates (22.5 mm diameter and 1 mm thickness). The cells were passaged at an 80% confluence. At passage three, cells were seeded at a density of 50,000 cells/cm² in 3 mL of media. The hydrogels were then incubated, and cyclic loading (−0.5 to −3 N at 2 Hz) was initiated for the duration of the test (as presented in section 2.3.1). A group of cells were supplemented with an osteocyte differentiation toolkit (PCS-500-052) and used as the positive control (FIG. 12). A group of cells was seeded in the well plate without hydrogel as a reference. The medium was changed every 48 h. After 7 and 14 days of culture, the cells were evaluated for viability, gene expression, and mineralization.

### Cell Viability Test

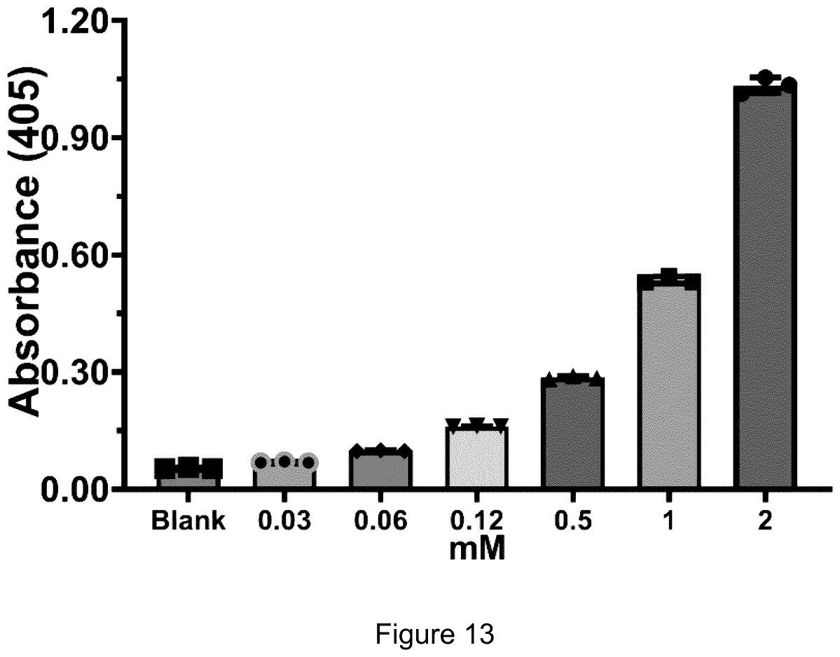

Cellular metabolic activity was assessed using alamarBlue (Invitrogen A50100) (Invitrogen, alamarBlue HS Cell Viability Reagent, 2019, 1-2). After 7 and 14 days of incubation, the medium was removed (N=3 samples per group), and 10% (v/v) alamarBlue was added. The plates were incubated for an additional 4 h. Next, aliquots of the solution (100 μL) were transferred to a 96-well plate. Absorbance at 570 nm (A570 to detect oxidized alamarBlue) and 600 nm (A600 to detect reduced alamarBlue) was measured to calculate the reduction of alamarBlue as instructed by the manufacturer (Invitrogen, alamarBlue HS Cell Viability Reagent, 2019, 1-2). To estimate the cell viability, the alamarBlue reduction values were normalized by the readings obtained from wells containing only cells as a baseline measurement (FIG. 13) (Posimo et al., 2014, J. Vis Exp., e50645).

### Molecular Evaluation of Bone Regeneration: RNA Extraction and Gene Expression Assays

The hydrogels with the cell monolayer were transferred to a 50 mL centrifuge tube. Direct cell lysis was performed using 600 μL of RTL buffer containing 1% (v/v) β-mercaptoethanol (RNeasy Mini Kit, Qiagen), followed by vortexing for 1 min (Svec et al., 2013, Front. Oncol., 3:1-11). RNA was extracted from the resulting cell lysate, followed by cDNA synthesis and RT-qPCR analysis as described above. Gene-specific primers associated with osteoblast differentiation were analyzed, including ALP, COL1A1, and RUNX2. GAPDH was used as the internal control (Table 2) (Stephens et al., 2011, BMC Res. Notes, 4:410).

TABLE 2
Primer sequences used for RTq-PCR analysis of BMSCs cells.

| Name | Function | Primer Sequence |
|---|---|---|
| GAPDH | Housekeeping | F 5′-3′: GTCTCCTCTGACTTCAACAGCG |
| | | R 5′-3′: ACCACCCTGTTGCTGTAGCCAA |
| COL1A1 | Major component of the organic | F 5′-3′: ATGACTATGAGTATGGGGAAGCA |
| | bone matrix | R 5′-3′: TGGGTCCCTCTGTTACACTTT |
| ALP | Involved in the mineralization | F 5′-3′: ATGGGATGGGTGTCTCCACA |
| | process | R 5′-3′: CCACGAAGGGGAACTTGTC |
| RUNX2 | Associated with osteogenic | F 5′-3′: ATTCCTGTAGATCCGAGCACC |
| | differentiation | R 5′-3′: ATTCCTGTAGATCCGAGCACC |

F: Forward primer.
R: reverse primer.

Gene expression was normalized using the comparative Ct (ΔΔCt) method (Rao et al., 2013, Biostat. Bioinf. Biomath., 3:71). Each experimental group and gene primer were measured in triplicates.

### Mineralization Assay

Mineralization was measured on day 14. First, the cells were fixed with 4% formalin for 15 min at room temperature and stained with Alizarin Red S (ARS) (40 mM, pH 4.2) (ScienCell), followed by incubation at 37° C. for 10 min (Hatt et al., 2019, Int. J. Mol. Sci., 20:5801). The stained samples were diluted with 800 μL of 10% (v/v) acetic acid and neutralized with 200 μL of 10% ammonium hydroxide for 30 min. Optical density was measured at 405 nm (A405) using a Multiskan SkyHigh spectrophotometer. Before the experiment, a calibration curve was constructed to validate ARS measurements (FIG. 13) (Moser et al., 2018, Front. Bioeng. Biotechnol., doi: 10.3389/fbioe.2018.00161). The absorbance results were normalized to the cell growth area within the hydrogels, and each experimental group was evaluated in triplicates.

### Periodontal Bone-Tissue Regeneration and Inflammatory Evaluations In Vivo

### Animals and Procedures

The treatment of PD using PiezoGEL was evaluated using an established ligature-induced mice model (Xu et al., 2019, Acta Biomater., 86:235-246; Tsukasaki et al., 2018, Nat. Commun., 9:1-11; Marchesan et al., 2018, Nat. Protoc., 13:2247-2267). All animal experiments were reviewed and approved and complied with institutional, state, and federal policies. Female C57BL/6 mice (N=20) (Charles River Laboratories, Malvern, USA) weighing approximately 25 g (8-12 weeks of age) were obtained. To induce PD and bone loss, animals were anesthetized with 4% (v/v) isoflurane. A 5-0 silk ligature (Roboz Surgical Instrument, SUT151) was tied around the left maxillary second molar for 8 days (FIG. 5A) (de Molon et al., 2018, Microsc. Res. Technol., 81:1412-1421).

To initiate treatment, animals were anesthetized with 4% (v/v) isoflurane. Clinical examination was performed by measuring the probing pocket depth using a Williams probe. Five mice were randomly assigned to each experimental group, positive control (Healthy), PiezoGEL, GelMA, and SRP. Four animals were included in the no treatment group. Before treatment, SRP was performed inside the defect using a dental Gracey Curette (Micro Mini Five #18). The hydrogels were carefully injected into the periodontal defect using a micropipet containing approximately 10 μL of the uncured hydrogel. Then, the hydrogel was cured for 20 s using a 405 nm visible light positioned at 1 cm from the material (total radiant exposure of 3.1 J/cm²). After one month of treatment, the mice were euthanized by CO₂ inhalation. Collected tissue samples were fixed in 10% formalin for 24 h at 4° C. and subsequently transferred to 80% ethanol for further analysis according to established protocols (Marchesan et al., 2018, Nat. Protoc., 13:2247-2267).

### Periodontal Bone-Tissue Regeneration Assessment

Two methods were used to assess the bone tissue regeneration performance. First, the probing pocket depth (PPD) was measured before and after treatment by using a periodontal probe. The change in pocket depth was calculated by subtracting the before and after treatment measurements and normalized to the initial defect depth (day 0). Second, microtomography (micro-CT) was used to obtain a 3D reconstruction of periodontal hard tissues and measure the changes in bone tissue. Briefly, the fixed maxillae were scanned using a scanner (Bruker Skyscan 1172 XR) operated at an accelerated potential of 65 kV, a beam current of 156 μA, an exposure time of 430 ms, and an imaging resolution of 18 μm. All scans were aligned along the sagittal, transverse, and coronal planes, ensuring that all images had a consistent orientation (Data Viewer software). The scanned DICOM images were reconstructed into a 3D image (Nrecon software). Micro-CT reconstruction was analyzed according to established protocols (Bouxsein et al., 2010, Journal of Bone and Mineral. Research, 25:1468-1486). The brightness and contrast of all images were adjusted through histogram standardization (0.00-0.15) to enhance accuracy and enable automated algorithmic analysis and image comparison (Zong et al., 2022, Sci. Rep., 12:1-13).

To assess the amount of regenerated bone tissue, the standard distance between the CEJ and ABC between M1 and M2 (defect location) was measured (Marchesan et al., 2018, Nat. Protoc., 13:2247-2267). To assess the regenerated bone tissue volume, the percentage of bone tissue was measured in selected areas over four planes spaced by 90 μm along the occlusal/apical direction (Chavez, 2021, JBMR Plus, doi: 10.1002/jbm4.10474). Each selected area (100×300 μm²) was consistently analyzed between M1 and M2. The percentage of bone tissue of each area and plane was added. A healthy mouse with no defect or disease was considered as 100% for comparison.

### Histological Assessment

The maxillae were decalcified in 25% formic acid for 48 h after fixation. Samples were embedded in paraffin, and 5 μm thickness sections perpendicular to the long axis of the teeth were obtained. Histological sections were stained with hematoxylin-eosin (H&E) and examined by light microscopy (Nikon Eclipse 50i microscope). The slides were digitally scanned using the ScanScope CS system (AperioScan Technologies, Vista, CA, USA) and ImageScope viewing software. Histological measurements included blood vessel count, erythrocyte count, epithelial length (FIG. 14), lymphoplasmacytic infiltration, and congestion (FIG. 15) (Batool et al., 2018, Nanomaterials, 8:337; Chen et al., 2021, Cell Prolif., 54:e12973).

### Statistical Analysis

For all evaluations, statistical differences were determined using one-way analysis of variance (ANOVA) with a significance level of 0.05. The ANOVA assumption of normal distribution was assessed by the Shapiro-Wilk test, and the assumption of homoscedasticity was assessed using Levene's test. Tukey's post hoc test was used for multiple comparisons with a 95% confidence level. Statistical analyses were performed using GraphPad Prism 9 software (GraphPad Software, California, USA). Samples for three different batches were used for experimentation.

### Example 2

### Material Selection

To find the appropriate BTO and GelMA concentrations of PiezoGEL, preliminary tests, including biodegradation, voltage generation, and injection force, were conducted. Hydrogels were fabricated and evaluated with different concentrations of GelMA (100 mg/mL and 200 mg/mL) and BTO (3 mg/mL, 6 mg/mL, and 9 mg/mL). Hydrogels were fabricated, as explained in Example 1.

To select the GelMA concentration, biodegradation evaluations were conducted in vitro. Two concentrations of GelMA with 100 mg/mL (10% wt) and 200 mg/mL (20% wt) filled with 9 mg/mL of BTO were fabricated. As controls, GelMA with similar concentrations but without BTO were also fabricated. Discs of 12 mm diameter and 6 mm thickness were obtained. Samples were submerged in PBS for 7 and 14 days at 37° C. FIG. 7 showed the biodegradation profiles for the 4 formulations. Overall, the addition of BTO did not influence the degradation profile. However, GelMA with different concentrations (10% versus 20%) showed different degradation profiles. Overall, a lower concentration of GelMA resulted in increased degradation. After 14 days, a GelMA concentration of 100 mg/mL (10%) resulted in 80% degradation compared to 40% in the 200 mg/mL (20%). For PD treatment, higher degradation rates of the hydrogel may not provide sufficient mechanical support for the new bone tissue to form, in addition to the early exhaustion of the biomaterial leading to weak bone regeneration and continued inflammation (Sung et al., 2004, Biomaterials, 25:5735-5742). Therefore, GelMA with 200 mg/mL was selected.

To select the BTO concentration, piezoelectric performance (i.e., minimum electrical charge required to enable bioactive effects) and injectability evaluations were conducted. Previous studies revealed the effect of BTO concentration and the magnitude of electrical charges on the antimicrobial response (Montoya et al., 2021, ACS Appl Mater Interfaces, 13:43868-43879; Montoya et al., 2021, ACS Biomater Sci Eng., 7:4838-4846). The best antibacterial effect of piezoelectric charges was found for lower amounts of BTO (<10%) and small voltages (10 mV/cm²) after testing a BTO composite against Streptococcus mutans (gram-positive bacteria).

Discs of 12 mm diameter and 6 mm thickness were fabricated with different concentrations of BTO including 3 mg/mL, 6 mg/mL, 9 mg/mL. The voltage generated by the hydrogels was measured as explained in Example 1. Overall, the obtained voltage was proportional to the amount of the piezoelectric filler (FIG. 8A). As expected, no voltage was detected in the GelMA material since there was no BTO filler. The highest obtained voltage (12 mV/cm²) was measured for the 9 mg/mL concentration. This magnitude of electrical charges has shown acceptable antimicrobial response in previous studies.

To verify that the addition of BTO fillers into GelMA did not significantly increase the injection force, the injectability tests were conducted as explained in Example 1. Using a syringe (1 mL) connected to a 20-G tip and filled with liquid PiezoGEL with different BTO concentrations, it was observed that adding 3 mg/mL and 6 mg/mL of BTO to GelMA does not significantly affect the injection force of the material (FIG. 8B). An average injection force of 8.5 N (0.9 kgf) was measured. The addition of a higher BTO quantity (9 mg/mL) resulted in a significant increase in the injection force compared to the GelMA (12 N-1.2 kgf) (FIG. 8B). All injections forces were acceptable for clinical use (<3 kgf) (Alonso et al., 2021, Polymers (Basel), 13:1-24).

### Surface Pore Size

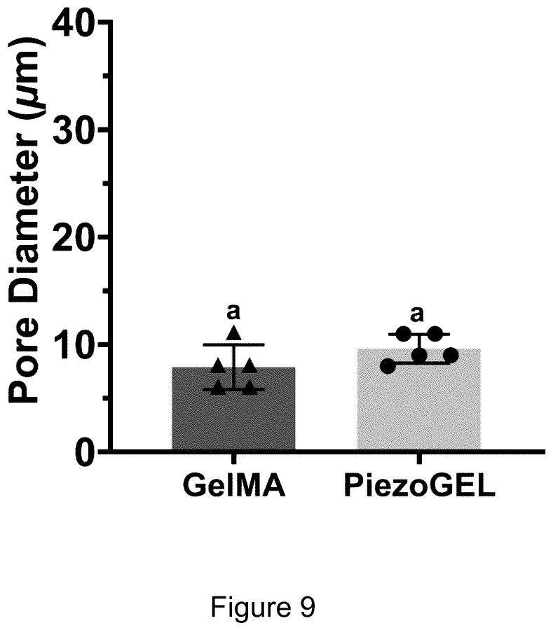

Surface porosity can have a significant effect on cell adhesion. The presence of pores or void spaces within a material affects the ability of cells to attach, spread, and interact with the material's surface (Dubruel et al., 2007, Biomacromolecules, 8:338-344). The surface pore size of the hydrogels was examined using an optical microscope (Olympus BX-41, Japan). Twenty samples (10 per group) were analyzed. Pore size was measured using the ImageJ software. The average pore sizes were found to be 10.2 μm for PiezoGEL and 10.94 μm for the GelMA hydrogels (FIG. 9). The mean pore sizes of both samples were not significantly different (p≤0.05).

### Surface Roughness

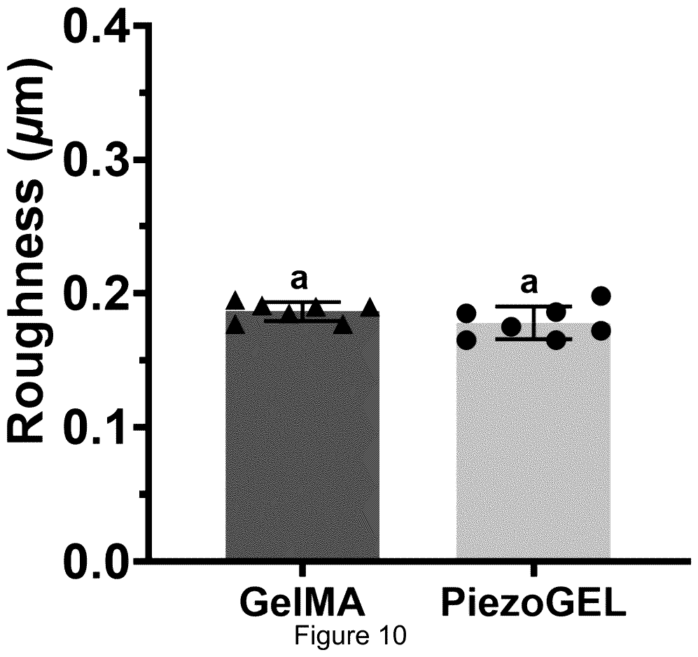

Surface roughness plays an important role in the adhesion and growth of bacterial biofilms. Microbial colonization increases as the surface roughness increases (Yang et al., 2022, J Mater Sci Technol., 99:82-100; Donlan et al., 2002, Biofilms: Microbial Life on Surfaces, 8:881-890). In contrast, roughness less than 0.2 μm has not been shown to affect cell attachment or biofilm formation (Montoya et al., 2021, ACS Appl Mater Interfaces, 13:43868-43879; Van Bollen et al., 1996, Clin Oral Implants Res., 7:201-211). A contact profilometer (Surfcorder SE 1700) was used to measure the mean roughness (Ra) at the surface of the hydrogels. Each sample was measured in 3 randomly selected locations. Only samples with an average roughness lower than 0.2 μm were used in the following evaluations. The mean surface roughness for all samples (FIG. 10) was lower than 0.2 μm. No significant differences were measured between PiezoGEL and GelMA hydrogels.

### Live and Dead Fluorescent Microscopy

To visualize and quantify the live and the dead bacteria attached on the hydrogels, bacterial cells were stained with a SYTO9/propidium iodide (PI) solution. The fluorescent SYTO9 stains live cells with intact cell membranes, whereas dead cells with damaged membranes are stained with the PI. To quantify the proportion of live and dead bacteria, color images were converted to 8-bit images, and the threshold was set from 0 to 255 (FIG. 11). Then, a similar threshold was applied to obtain black and white images. All pixels with values above the threshold (white pixels) were counted. The number of white pixels was computed separately for both live and dead images. As a control, cells treated with H₂O₂ were also imaged (80-100% dead cells).

BMSC Culture with Positive Control (Differentiation Media)

To compare the cell differentiation capability of PiezoGEL with differentiation media, groups of cells were cultivated as explained in Example 1. An additional group (Ctrl+) was supplemented with an osteocyte differentiation toolkit (PCS-500-052). The media was changed every third day. After 7 and 14 days of culture, the cells were evaluated for viability, gene expression, and mineralization (as explained earlier). In the alamarBlue results, as shown in FIG. 12B, hydrogels showed higher cell viability than the Ctrl+. No significant differences were seen between day 7 and 14. Looking into the expression of early markers of osteogenic differentiation, relevant BMSC genes, including RUNX2, COL1A1, and ALP were examined (FIG. 12C through FIG. 12E). On day 7, the Ctrl+ exhibited higher gene expression than the GelMA groups (static or cyclic) but lower than the PiezoGEL. On day 14, a similar gene expression was measured for the Ctrl+ and PiezoGEL. The Alizarin Red S assay revealed that the differentiating group had the second-highest absorbance after the cyclical PiezoGEL, demonstrating significant mineralization potential with the differentiating medium (FIG. 12F).

### Alizarin Red S Standard Curve

Mineralization capability of the hydrogels was evaluated with Alizarin Red S (ARS). To determine the Alizarin Red S concentration in millimole (mM), a standard curve was prepared by dissolving different concentrations of ARS in a solution composed of 20% methanol and 10% acetic acid in water. Photometric absorption was determined at 405 nm. As presented in FIG. 13, the measured absorbance is proportional to the measured absorbance.

### Epithelial Length

Epithelial length is an important variable for evaluating the inflammatory response. Prolonged inflammation can lead to epithelial hyperplasia, increasing epithelial length (Shukla et al., 2014, J Indian Soc Periodontol., 18:92). Histological images were used to measure changes in the epithelial length. For each group, 6 measurements were made. As shown in FIG. 14, a higher epithelial length was measured for the no treated animals, followed but the GelMA hydrogel. The lowest epithelial length was measured for PiezoGEL and the SRP groups.

### Inflammation Assessment

Two-blinded pathologists performed an inflammation assessment of the affected tissues. The evaluation was performed in terms of lymphoplasmacytic infiltration and congestion. Lymphoplasmacytic infiltration refers to the presence of lymphocytes and plasma cells in the histological examination of periodontal tissues (Popa et al., 2021, Rom J Morphol Embryol., 62:209-217). Higher infiltration of inflammatory cells is related to the triggering of the immune response due to the bacterial biofilm. Histologically, congestion refers to an abnormal accumulation of blood within blood vessels or tissues (Tan et al., 2020, Int J Clin Exp Pathol., 13:635). Each image was assigned a score from 0 (no lymphoplasmacytic infiltration or congestion) to 5 (severe lymphoplasmacytic infiltration or congestion). As shown in FIG. 15A, the highest score of lymphoplasmacytic infiltration was assigned to the no treatment and GelMA groups. Similar scores were found for the tissues treated only with SRP and PiezoGEL. As expected, the lowest infiltrate was observed in the healthy tissues. Regarding congestion, a similar score was assigned to all groups except for the animals treated with PiezoGEL and healthy (FIG. 15B).

### Colony-Forming Units Evaluation

The quantification of Colony-Forming Units (CFUs) on Blood sheep agar plates supplemented with hemin and menadione, is presented (FIG. 16). After 24 hr of culture, the groups GelMA and PiezoGEL (under both static and cyclic conditions) were evaluated using the same dilution. The lowest bacterial count was exhibited by the cyclic PiezoGEL group, indicating a significant reduction in bacterial growth compared to the other groups.

### Leaching of BTO Fillers

The application of cyclic loading to PiezoGEL continuously for more than 7 days can cause the leaching of the BTO nanoparticles to the cell growth media. To determine the leaching of nanoparticles, samples of PiezoGEL were incubated in Bone Marrow Stem Cells (BMSCs) growth medium (ATCCR, PCS-500-040) for 7 days under static and continuous cyclic loading. After 7 days, the media was removed and centrifugated for 5 min at 12×g. Later the supernatant was removed, and the remaining material was evaluated using a scanning electron microscope (FEI Quanta 450 EFG). FIG. 17A shows a representative image of the material found under cyclic loading and FIG. 17B under static conditions. Chemical evaluation of the materials observed in the micrograph was conducted by energy dispersion of Xray. FIG. 17C shows the EDS spectra. Clear peaks associated with barium and titanium are observed, corresponding to the composition of the BTO fillers.

### Periodontal Pocket Depth Measurements

Periodontal disease and bone loss were successfully induced in mice after placing a ligature around the molars for a week. An initial clinical evaluation for bleeding and periodontal pocket depth was performed on all infected animals. Healthy animals were evaluated as a control (FIG. 18). Healthy and diseased pocket depths were 0.2 and 0.55 mm, respectively. The results are consistent with previous studies showing that the pocket depth after 1 week of ligature-induced PD is ~0.60 mm (Li et al., 2020, Front Bioeng Biotechnol., 8:444).

### Example 3: Fabrication of Light Curable 20% GelMA Hydrogels Filled with 9 mg/mL of Barium Titanate (BTO) Nanoparticles for the Treatment of Periodontal Disease

The present studies prepared 250 mg of 20% GelMA (1.25 mL). First, it was required to comply with aseptic practices to maintain the sterility of the hydrogel throughout the fabrication. Wearing nitrile gloves throughout the procedure and using alcohol to disinfect them regularly was required. The biosafety cabinet was then turned on and 70% ethanol was sprayed on all surfaces. The door of the biosafety cabinet were closed and the UV light was turned on to disinfect (10 minutes). All the materials needed were placed inside the biosafety cabinet and sprayed with 70% ethanol. The extraction system of the biosafety cabinet was checked to ensure it works correctly. The heater of the Ultrasonic bath sonicator (Fisher CPX28000) was turned on and set the temperature to 70° C. Once the water temperature stabilized and reached 70° C., the water temperature was verified with a thermometer (takes around 15 minutes).

200 mL beaker was then placed in the sonicator bath and fixed with a stable support. The beakers was filled with MilliQ water. 250 mg vial of GelMA was wrapped with aluminum foil to prevent exposure to light. Using a sterile serological pipet, 3 mL of sterile PBS was added into a 15 mL centrifuge tube. PBS was heated to 70° C. by placing the centrifuge inside the beaker until the temperature stabilized. Once the PBS reached 70° C., the lid of the GelMA vial and the PBS centrifuge tube were open. Using a sterile 1000 μL pipette with tip, 1.25 mL of PBS was aspirated and dispensed inside the GelMA vial. The vial lid was then closed and kept at 70° C. inside the sonicator until complete solubilization was observed. Using a disinfected spatula and a precision balance, the weight of the following materials was measured: 9 mg/mL of BTO (for 1.25 mL of PBS this will be 11.25 mg of BTO) and 2.5 mg LAP. 11.25 mg of BTO was then placed inside the GelMA vial and vortexed for 30 seconds at 3,200 rpm.

The required amount of LAP was solubilized in 50 μL of sterile PBS and mixed by pipetting 5-6 times. This procedure was performed in the dark. The solution was then sonicated for 1 h at 70° C. and 40 kHz, and then immediately transferred into a syringe using a 1 mL sterile pipette and tips. The syringe was wrapped with aluminum foil and allowed to cool down at ambient temperature. Shortly after reaching room temperature, the syringe was stored in a refrigerator (−2 to 4° C.). Batch was labeled as needed.

PiezoGEL needed to be transported in dry ice to ensure the efficiency of the product. Any transport should be made in overnight shipping.

### Example 4: PiezoGEL™ Promoted Regrowth of High-Quality Bone

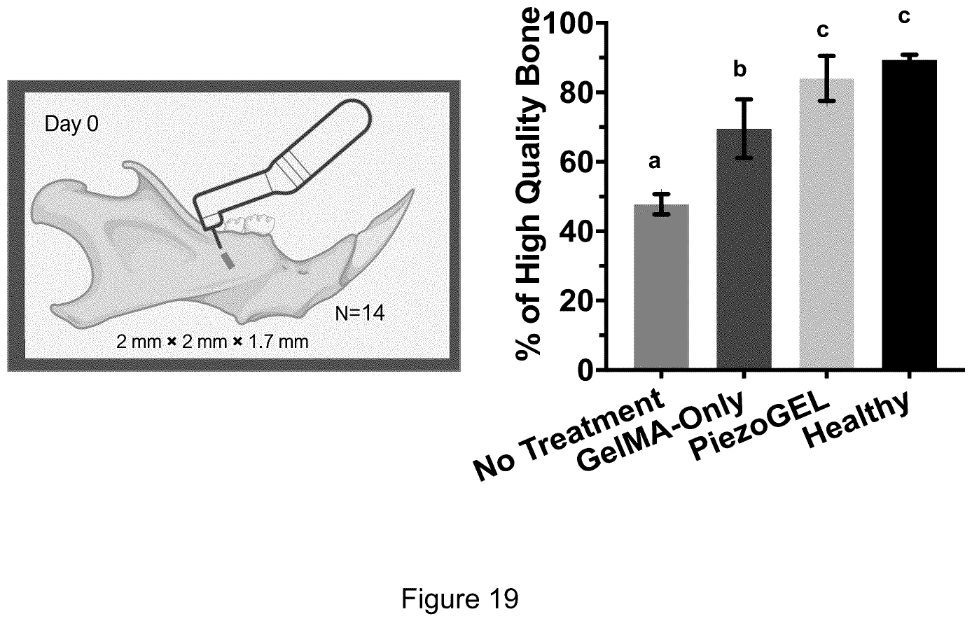

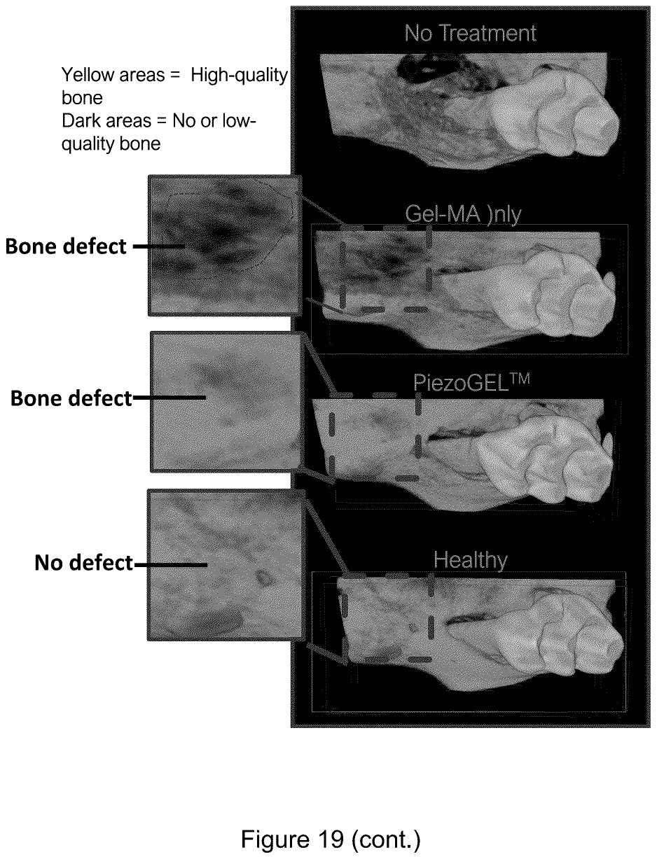

As shown in FIG. 19, bone defect model was used to demonstrate that PiezoGEL™ promoted regrowth of high-quality bone.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a polymer matrix and a piezoelectric material, wherein the piezoelectric material is dispersed within the polymer matrix.

2. The composition of claim 1, wherein the piezoelectric material comprises BaTiO₃ (BTO) or a derivative thereof.

3. The composition of claim 1, wherein the composition comprises between about 0.01% w/v to about 50% w/v of the piezoelectric material.

4. The composition of claim 1, wherein the polymer matrix comprises a hydrogel.

5. The composition of claim 4, wherein the hydrogel comprises methacrylate-containing hydrogel, acrylate-containing hydrogel, gelatin-based hydrogel, functionalized gelatin-based hydrogel, or any combination thereof.

6. The composition of claim 5, wherein the hydrogel comprises gelatin methacrylate or an analog thereof, gelatin acrylate or an analog thereof, or any combination thereof.

7. The composition of claim 1, wherein the composition
a) has a shear storage modulus (G′) between about 10² Pa to about 10⁴ Pa;
b) has a shear loss modulus (G″) between about 10² Pa to about 10³ Pa;
c) has a voltage per surface area of at least about 9 mV/mm²;
d) has an injection force of less than about 3 kgf;
e) has a flow rate of about 2 mL/h;
f) comprises at least one pore having an average diameter of at least about 10 μm;
g) comprises a surface roughness of less than 0.2 μm;
h) has curing time of between about 15 seconds to about 180 seconds;
i) has a degradation rate of at least about 5% over about 14 days; or
any combination thereof.

8. The composition of claim 1, wherein the composition further comprises a nanoparticle, initiator, stabilizer, light emitting component, thermal emitting component, therapeutic agent, polymer, binding agent, linking molecule, or any combination thereof.

9. The composition of claim 8, wherein the nanoparticle comprises silicon oxide nanoparticle, silanized nanoparticle, silicon-free nanoparticle, or any combination thereof.

10. The composition of claim 8, wherein the initiator comprises lithium phenyl-2,4,6-trimethylbenzoyl phosphonate (LAP) or a derivative thereof.

11. The composition of claim 1, wherein the composition is a light emitting component-free composition, thermal emitting component-free composition, or a combination thereof.

12. The composition of claim 1, wherein the composition is a biocompatible composition, biodegradable composition, antimicrobial composition, or any combination thereof.

13. The composition of claim 1, wherein the composition is a dental composition.

14. The composition of claim 1, wherein the piezoelectric material promotes bone growth, prevents, reduces, or treats bone damage, prevents, reduces, or treats bone structure damage, prevents, reduces, or treats tooth damage, prevents, reduces, or treats tooth structure damage, prevents, reduces, or treats an infection, prevents, reduces, or treats an inflammation, prevents or reduces biofilm formation, or any combination thereof.

15. The composition of claim 14, wherein the infection is a bacterial infection, fungal infection, viral infection, or any combination thereof.

16. A method of preventing or treating an oral disease, disorder, or condition in a subject in need thereof, wherein the method comprises applying at least one composition of claim 1 to at least a portion of a tooth of the subject.

17. A method of simultaneously preventing or treating an oral disease, disorder, or condition and promoting mineralization in a subject in need thereof, wherein the method comprises applying at least one composition of claim 1 to at least a portion of a tooth of the subject.

18. The method of claim 16, wherein the oral disease, disorder, or condition is periodontal disease, peri-implantitis, oral infection, loss of tooth structure, gum inflammation, gingivitis, or any combination thereof.

19. The method of claim 18, wherein the oral infection is a bacterial infection, fungal infection, viral infection, or any combination thereof.

20. The method of claim 16, wherein the subject generates mastication forces producing electrical charges at the surface of the composition, wherein the electrical charges simultaneously prevent or reduce microbial growth and precipitate new minerals.
